(19) United States
(12) Patent Application Publication
(10) Pub. No.: US 2011/0262446 A1
(43) Pub. Date: Oct. 27, 2011

(54) USE OF IGF-II/IGF-IIE BINDING PROTEINS FOR THE TREATMENT AND PREVENTION OF SYSTEMIC SCLEROSIS ASSOCIATED PULMONARY FIBROSIS

(75) Inventor: Edward H. Cohen, Belmont, MA (US)

(73) Assignee: DYAX CORP., Cambridge, MA (US)

(21) Appl. No.: 13/124,044

(22) PCT Filed: Oct. 14, 2009
(86) PCT No.: PCT/US09/60627

§ 371 Date: Jul. 11, 2011

Related U.S. Application Data
(60) Provisional application No. 61/105,229, filed on Oct. 14, 2008.

(51) Int. Cl. A61K 39/395; A61P 11/00

(52) U.S. Cl. 424/139.1; 424/158.1

(57) ABSTRACT

Methods of using proteins that bind to IGF-II and/or IGF-IIE for the treatment or prevention of systemic sclerosis-associated pulmonary fibrosis are described.

### CROSS REFERENCE TO RELATED APPLICATIONS

This application claims priority to U.S. Application Ser. No. 61/105,229, filed on Oct. 14, 2008. The disclosure of the prior application is considered part of (and is incorporated by reference in) the disclosure of this application.

### BACKGROUND

The insulin-like growth factor (IGF) family of polypeptides plays a key role in normal growth and development. Altered expression of components, such as IGF-II, of the IGF system are implicated in the development and maintenance of the malignant phenotype in many tumor types, suggesting that agents targeting this system may have potential as anti-cancer therapeutics. A number of pathways have been identified that can contribute to increased IGF-II secretion by tumors; these include a loss of genomic imprinting of the maternal IGF-II allele, loss of heterozygosity with paternal allelic duplication, and/or loss of transcriptional control. Such increased secretion allows for greater proliferation, protection from apoptosis and metastatic potential of a cancer, especially as the receptors that specifically bind the IGF-II, the IGF-I receptor (IGF-1R or IGF-IR) and an isoform of the insulin receptor (IR-A), are typically up-regulated in tumor cells. The increased production of IGF-II is further exacerbated by the down-regulation of the mannose-6-phosphate receptor, a third type of IGF-II receptor that appears to be central for the clearance of IGF-II from the circulation. Local levels of IGF-II may also be elevated by changes in the expression of specific IGF-II binding proteins secreted by the tumor, or as a result of increased protease activity produced by the tumors.

Even though it is implicated in the pathogenesis of 50% of cancers, there are limited therapeutic agents available that specifically target the IGF signaling axis although there are many research efforts underway to remedy this. It has recently been demonstrated that the efficacy of EGF receptor antagonists in a model of breast cancer treatment is limited by the rapid development of resistance via the IGF system. Discovery or development of therapeutics that interfere with the IGF system is complicated by the finding that most IGF-I receptors appear to form hybrid receptors with the IR-A, the isoform of the insulin receptor that binds both IGF-II and insulin with high affinity. Therefore, therapeutic targeting of the IGF-IR with tyrosine kinase inhibitors or antibodies may also block insulin signaling and cause diabetes, and recent reports have indicated this to be the case. Toxicity problems with the IGF-IR small molecule kinase antagonists in primate studies have also been reported.

In normal circulation, 95% of IGF-I and II are bound to six high-affinity IGF binding proteins (IGFBPs). The major serum-binding protein is IGFBP-3, which forms a trimeric complex with acid labile protein (ALS). Normally, IGF-II is synthesized as a 156 amino acid (aa) precursor protein, known as pro-IGF-II. This protein includes an 87 aa C-terminal region known as the E-domain, and is thus referred to as “IGF-IIE.” Herein, the construct comprising amino acids 1-104, which encompass the E domain, is referred to as “IGF-IIE”. Proteolytic steps release the mature 67 aa IGF-II polypeptide. In the literature, “long” or “big” forms of IGF-II sometimes refer to forms in which only portions of the E domain are cleaved. Sometimes the long or big forms are also referred to as IGF-IIE even though they may contain only parts of the E domain as opposed to the complete E domain.

In many tumors, there is increased production of IGF-II, due mainly to loss of imprinting at a genomic level, or decreased levels of binding proteins due to increased protease activity produced by the tumors that allow for increased bioavailability of free IGF-II. In a recent IGF II mouse model, offspring mice with loss of imprinting characteristics, mated with Apc+/Min mice, have shown greatly enhanced tumorigenesis. Many tumors lack the enzymatic machinery for processing IGF-IIE to the mature 7.5 KDa protein and thus predominately secrete IGF-IIE. This long IGF-II ligand (amino acids 1-104) has a 21 amino acid extension at the carboxy terminus and defective glycosylation at threonine 75 and therefore cannot bind ALS, allowing greater “free IGF-II” to activate the IR or IGF-I R, potentiating neoplastic growth and, in some cancers, causing hypoglycemia.

### SUMMARY

Lung fibroblasts appear to be a major source of IGF-II, and there are significant increases in IGF-II RNA and protein expression in primary lung fibroblasts from systemic sclerosis (SSc) lungs as compared to normal lung fibroblasts. Furthermore, primary SSc lung fibroblasts have approximately a four-fold increase in IGF-II mRNA and a two-fold increase in IGF-II protein compared with normal lung fibroblasts. IGF-II mRNA in SSc lung fibroblasts is expressed primarily from the P3 promoter of the IGF-II gene, and IGF-II induces both a dose- and time-dependent increase in collagen type I and fibronectin production. IGF-II can trigger the activation of both phosphatidylinositol-3 (PI3) kinase and Jun N-terminal kinase (JNK) signaling cascades, and Akt phosphorylation in lung fibroblasts Inhibitors of PI3 kinase and JNK can block IGF-II induced production of collagen and fibronectin. The addition of IGF-II to SSc lung fibroblasts can significantly increase collagen (e.g., collagen I) and fibronectin production, e.g., in a dose-dependent manner, while only minimally altering extracellular matrix (ECM) production in normal lung fibroblasts. SSc lung fibroblasts can produce statistically more collagen type I at ≧10 ng/ml exogenously added IGF-II and fibronectin at ≧100 ng/ml exogenously added IGF-II than normal lung fibroblasts.

Increased collagen and/or fibronectin production, hallmarks of systemic sclerosis-associated pulmonary fibrosis, can be inhibited by blocking the interaction between IGF-II, and/or possibly IGF-IIE (a long IGF-II), to an IGF receptor, e.g., the IGF-I receptor.

Therapeutic treatment (e.g., immunotherapeutic treatment) of systemic sclerosis (SSc)-associated pulmonary fibrosis can provide advantages over traditional therapies such as immunosuppression and/or surgery, as the therapeutic agent (e.g., the cytokine, antibodies or antibody-like moieties) can be highly specific for lung fibroblasts, e.g., lung fibrolasts in SSc lungs, or fibroblastic foci. A therapeutic agent targeting IGF-II would block the action of this ligand by inhibiting binding to both the IGF-I receptor and IR-A, without causing down regulation of the IR and the potential risk of hypoglycemia and/or diabetes. Development of a binding protein, e.g., an antibody, that specifically binds IGF-II and IGF-IIE would also alleviate the issue of toxicity demonstrated with kinase antagonists. Further, a therapeutic agent targeting only IGF-IIE and not IGF-II would also be of value.

Accordingly, this disclosure relates, inter alia, to a method of treating or preventing systemic sclerosis-associated pulmonary fibrosis in a subject, the method comprising:

administering an isolated protein (e.g., antibody, e.g., human antibody) that binds IGF II and/or IGF IIE to the subject, wherein the antibody binds the same epitope or competes for binding with an antibody selected from the group consisting of DX-2647, M0033-E05, M0063-F02, M0064-E04, M0064-F02, M0068-E03, M0070-H08, M0072-006, M0072-E03, M0072-G06, germlined M0064-E04, germlined M0064-F02, and DX-2655.

In some embodiments, the antibody competes with or binds the same epitope as DX-2647.

In some embodiments, the antibody competes with or binds the same epitope as M0064-F02.

In some aspects, the disclosure provides a method of treating or preventing systemic sclerosis-associated pulmonary fibrosis in a subject, the method comprising:
administering an isolated protein (e.g., antibody, e.g., human antibody) comprising a heavy chain immunoglobulin variable domain sequence and a light chain immunoglobulin variable domain sequence to the subject, wherein:
the heavy chain immunoglobulin variable domain sequence comprises one, two, or three (e.g., three) CDR regions from the heavy chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or
the light chain immunoglobulin variable domain sequence comprises one, two, or three (e.g., three) CDR regions from the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively), and
the protein binds to (e.g., and inhibits) both IGF-II and IGF-IIE.

In some embodiments, the one, two, or three (e.g., three) CDR regions from the heavy chain variable domain are from DX-2647 and/or the one, two, or three (e.g., three) CDR regions from the light chain variable domain are from DX-2647.

In some embodiments, the heavy chain immunoglobulin variable domain sequence comprises the heavy chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain immunoglobulin variable domain sequence comprises the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

In some embodiments, the heavy chain immunoglobulin variable domain sequence comprises the heavy chain variable domain of DX-2647, and/or the light chain immunoglobulin variable domain sequence comprises the light chain variable domain of DX-2647.

In some embodiments, the protein comprises the heavy chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

In some embodiments, the protein comprises the heavy chain of DX-2647, and/or the light chain of DX-2647.

In some aspects, the disclosure provides a method of treating or preventing systemic sclerosis-associated pulmonary fibrosis in a subject, the method comprising:
administering to the subject an isolated protein (e.g., antibody, e.g., human antibody) comprising a heavy chain immunoglobulin variable domain sequence and a light chain immunoglobulin variable domain sequence, wherein
the heavy chain immunoglobulin variable domain sequence comprises one, two, or three (e.g., three) CDR regions from the heavy chain variable domain of M0080-G03 or M0073-C11, and/or
the light chain immunoglobulin variable domain sequence comprises one, two, or three (e.g., three) CDR regions from the heavy chain variable domain of M0080-G03 or M0073-C11 (respectively),
and the protein binds to (e.g., and inhibits) IGF-IIE but not IGF-II.

In some embodiments, the heavy chain immunoglobulin variable domain sequence comprises the heavy chain variable domain of M0080-G03 or M0073-C11, and/or the light chain immunoglobulin variable domain sequence comprises the light chain variable domain of M0080-G03 or M0073-C11 (respectively).

In some embodiments, the protein comprises the heavy chain of M0080-G03 or M0073-C11, and/or the light chain of M0080-G03 or M0073-C11 (respectively).

In some aspects, the disclosure provides a method of treating or preventing systemic sclerosis-associated pulmonary fibrosis in a subject, the method comprising:
administering to the subject an isolated protein (e.g., antibody, e.g., human antibody) capable of specifically binding to the following consensus sequence or a functional fragment thereof:

TXCGGXLVXXLXXXXXXXXFXXXXPXXRVXRXSRGXVEEXCFRXXXXXXX
XXY

wherein X is any amino acid.

In some embodiments, the protein is capable of specifically binding to the following consensus sequence or a functional fragment thereof:

SETLCGGELVDTLQFVCGDRGFYFSRPASRVSRRSRGIVEECCFRSCDLALLETYCATPA.

In some embodiments, the protein comprises a heavy chain immunoglobulin variable domain sequence and a light chain immunoglobulin variable domain, wherein:

the heavy chain immunoglobulin variable domain sequence comprises one, two, or three (e.g., three) CDR regions from the heavy chain variable domain of DX-2647, M0033-E05, M0063-F02, M0064-E04, M0064-F02, M0068-E03, M0070-H08, M0072-C06, M0072-E03, M0072-G06, germlined M0064-E04, germlined M0064-F02, or DX-2655, and/or

the light chain immunoglobulin variable domain sequence comprises one, two, or three (e.g., three) CDR regions from the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively), and

the protein binds to (e.g., and inhibits) both IGF-II and IGF-IIE.

In some embodiments, the one, two, or three (e.g., three) CDR regions from the heavy chain variable domain are from DX-2647 and/or the one, two, or three (e.g., three) CDR regions from the light chain variable domain are from DX-2647.

In some embodiments, the heavy chain immunoglobulin variable domain sequence comprises the heavy chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain immunoglobulin variable domain sequence comprises the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

In some embodiments, the heavy chain immunoglobulin variable domain sequence comprises the heavy chain variable domain of DX-2647, and/or the light chain immunoglobulin variable domain sequence comprises the light chain variable domain of DX-2647.

In some embodiments, the protein comprises the heavy chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

In some embodiments, the protein comprises the heavy chain of DX-2647, and/or the light chain of DX-2647.

This disclosure relates, inter alia, to a method of treating or preventing systemic sclerosis-associated pulmonary fibrosis in a subject, the method comprising:

administering an isolated protein that binds IGF II and/or IGF IIE (e.g., an IGF-II/IGF-IIE binding protein described herein) to the subject, e.g., wherein the binding of the protein to IGF II and/or IGF IIE is characterized by an affinity of at least 10⁹ M⁻¹.

In some embodiments, the IGF-II/IGF-IIE binding protein is used in combination with a second therapeutic agent. In some embodiments, the second therapeutic agent is another IGF-II/IGF-IIE binding protein, e.g., another IGF-II/IGF-IIE binding protein described herein. In some embodiments, the second therapeutic agent is an anti-inflammatory drug (e.g., steroid), a cytotoxic drug, an immunosuppressive agent, a collagen synthesis inhibitor, or an endothelin receptor antagonist. For example, oral corticosteroids such as high doses of oral corticosteroids (e.g., prednisone, 40 to 80 mg daily) can be used. Cytotoxic drugs such as cyclophosphamide and immunosuppressants such as azathioprine (cyclophosphamide is also an immunosuppressant); or collagen synthesis inhibitors such as Pirfenidone or endothelin receptor antagonists such as Bosentan can be used as the second agent. In preferred embodiments, the second agent is cyclophosphamide or azathioprine. In preferred embodiments, the second agent is cyclophosphamide in combination with a small dose of a steroid; epoprostenol; bosentan; or iloprost (e.g, aerolized iloprost). In some embodiments, the IGF-II/IGF-IIE binding protein is used in combination with surgery, e.g., lung transplantation. In some embodiments, the second agent is another treatment for SSc-associated pulmonary fibrosis such as anti-inflammatory drug e.g., a steroid (e.g., a corticosteroid (e.g., prednisone)), a cytotoxic drug (e.g., cyclophosphamide), an immunosuppressant (e.g., cyclophosphamide or azathioprine), a collagen synthesis inhibitor (e.g., Pirfenidone), or an endothelin receptor antagonist (e.g., Bosentan).

In some embodiments, the IGF-II/IGF-IIE binding protein decreases collagen and/or fibronectin production by greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% as compared to a standard, e.g., the subject's collagen and/or fibronectin production before the treatment.

The protein can include one or more of the following characteristics: (a) a human CDR or human framework region; (b) the HC immunoglobulin variable domain sequence comprises one or more (e.g., 1, 2, or 3) CDRs that are at least 85, 88, 90, 92, 94, 95, 96, 97, 98, 99, or 100% identical to a CDR of a HC variable domain described herein; (c) the LC immunoglobulin variable domain sequence comprises one or more (e.g., 1, 2, or 3) CDRs that are at least 85, 88, 90, 92, 94, 95, 96, 97, 98, 99, or 100% identical to a CDR of a LC variable domain described herein; (d) the LC immunoglobulin variable domain sequence is at least 85, 88, 90, 92, 94, 95, 96, 97, 98, 99, or 100% identical to a LC variable domain described herein; (e) the HC immunoglobulin variable domain sequence is at least 85, 88, 90, 92, 94, 95, 96, 97, 98, 99, or 100% identical to a HC variable domain described herein; (f) the protein binds an epitope bound by a protein described herein, or an epitope that overlaps with such epitope; and (g) a primate CDR or primate framework region.

The protein can bind to IGF-II and/or IGF-IIE, e.g., human IGF-II and/or IGF-IIE, with a binding affinity of at least 10⁵, 10⁶, 10⁷, 10⁸, 10⁹, 10¹⁰ and 10¹¹ M⁻¹. In one embodiment, the protein binds to human IGF-II and/or IGF-IIE with a K_{off} slower than 1×10⁻³, 5×10⁻⁴ s⁻¹, or 1×10⁻⁴ s⁻¹. In one embodiment, the protein binds to human IGF-II and/or IGF-IIE with a Kₒₙ faster than 1×10², 1×10³, or 5×10³ M⁻¹ s⁻¹. In one embodiment, the protein inhibits both human IGF-II and human IGF-IIE activity, e.g., with a Ki of less than 10⁻⁵, 10⁻⁶, 10⁻⁷, 10⁻⁸, 10⁻⁹, and 10⁻¹⁰ M. In one embodiment, the protein inhibits either human IGF-II or human IGF-IIE activity, e.g., with a Ki of less than 10⁻⁵, 10⁻⁶, 10⁻⁷, 10⁻⁸, 10⁻⁹, and 10⁻¹⁰M. The protein can have, for example, an IC50 of less than 100 nM, 10 nM or 1 nM. For example, the protein may modulate IGF-I receptor (IGF-1R) and/or an isoform of the insulin receptor (IR-A) activity, as well as IGF-II and/or IGF-IIE. The protein may inhibit IGF-1R, IR-A, and IGF-II and IGF-IIE activity. The affinity of the protein for human IGF-II and/or IGF-IIE can be characterized by a K_{D} of less than 100 nm, less than 10 nM, or less than 1 nM.

In a preferred embodiment, the protein is an antibody (e.g., a human antibody) having the light and heavy chains of antibodies selected from the group consisting of M0033-E05, M0063-F02, M0064-E04, M0064-F02, M0068-E03, M0070-H08, M0072-C06, M0072-E03, M0072-G06, germlined M0064-E04, germlined M0064-F02, DX-2647, and DX-2655.

In a preferred embodiment, the protein is an antibody (e.g., a human antibody) having the heavy chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the light chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having light and heavy antibody variable regions of an antibody selected from the group consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a heavy chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a light chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs selected from the corresponding CDRs of the group of heavy chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs selected from the corresponding CDRs of the group of heavy chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655 and one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655 (respectively).

In a preferred embodiment, the protein is an antibody (e.g., a human antibody) having the light and heavy chains of DX-2647.

In a preferred embodiment, the protein is an antibody (e.g., a human antibody) having the heavy chain of DX-2647.

In a preferred embodiment, the protein is an antibody (e.g., a human antibody) having the light chain of DX-2647.

In a preferred embodiment, the protein is an antibody (e.g., a human antibody) having light and heavy antibody variable regions of an antibody selected from DX-2647.

In a preferred embodiment, the protein is an antibody (e.g., a human antibody) having a heavy chain antibody variable region of DX-2647.

In a preferred embodiment, the protein is an antibody (e.g., a human antibody) having a light chain antibody variable region of DX-2647.

In a preferred embodiment, the protein is an antibody (e.g., a human antibody) having one or more (e.g., 1, 2, or 3) heavy chain CDRs from the corresponding CDRs of the heavy chain of DX-2647.

In a preferred embodiment, the protein is an antibody (e.g., a human antibody) having one or more (e.g., 1, 2, or 3) light chain CDRs from the corresponding CDRs of the light chain of DX-2647.

In a preferred embodiment, the protein is an antibody (e.g., a human antibody) having one or more (e.g., 1, 2, or 3) heavy chain CDRs from the heavy chain of DX-2647 and one or more (e.g., 1, 2, or 3) light chain CDRs from the corresponding CDRs of the light chain of DX-2647.

In a preferred embodiment, the protein is an antibody (e.g., a human antibody) having the light and heavy chains of DX-2655.

In a preferred embodiment, the protein is an antibody (e.g., a human antibody) having the heavy chain of DX-2655.

In a preferred embodiment, the protein is an antibody (e.g., a human antibody) having the light chain of DX-2655.

In a preferred embodiment, the protein is an antibody (e.g., a human antibody) having light and heavy antibody variable regions of an antibody selected from DX-2655.

In a preferred embodiment, the protein is an antibody (e.g., a human antibody) having a heavy chain antibody variable region of DX-2655.

In a preferred embodiment, the protein is an antibody (e.g., a human antibody) having a light chain antibody variable region of DX-DX-2655.

In a preferred embodiment, the protein is an antibody (e.g., a human antibody) having one or more (e.g., 1, 2, or 3) heavy chain CDRs from the corresponding CDRs of the heavy chain of DX-2655.

In a preferred embodiment, the protein is an antibody (e.g., a human antibody) having one or more (e.g., 1, 2, or 3) light chain CDRs from the corresponding CDRs of the light chain of DX-2655.

In a preferred embodiment, the protein is an antibody (e.g., a human antibody) having one or more (e.g., 1, 2, or 3) heavy chain CDRs from the heavy chain of DX-2655 and one or more (e.g., 1, 2, or 3) light chain CDRs from the corresponding CDRs of the light chain of DX-2655.

In one embodiment, the HC and LC variable domain sequences are components of the same polypeptide chain. In another, the HC and LC variable domain sequences are components of different polypeptide chains. For example, the protein is an IgG., e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In some embodiments, the affinity of the primate antibody for human IGF-II and/or IGF-IIE is characterized by a K_{D} of less than 1 nM.

In certain embodiments, the protein includes no sequences from mice or rabbits (e.g., is not a murine or rabbit antibody).

In certain embodiments, the protein is capable of binding to lung fibroblasts, or fibroblastic foci, e.g., that express IGF-II and/or IGF-IIE.

In one embodiment, protein is physically associated with a nanoparticle, and can be used to guide a nanoparticle to a cell expressing IGF-II and/or IGF-IIE on the cell surface.

In other aspects, the disclosure relates to a method of treating or preventing systemic sclerosis-associated pulmonary fibrosis in a subject, the method comprising:

administering an isolated protein comprising a heavy chain immunoglobulin variable domain sequence and a light chain immunoglobulin variable domain sequence to the subject,

wherein the heavy chain immunoglobulin variable domain sequence comprises one or more (e.g., 1, 2, or 3) CDRs from the heavy chain of M0033-E05, M0063-F02, M0064-E04, M0064-F02, M0068-E03, M0070-H08, M0072-006, M0072-E03, M0072-G06, germlined M0064-E04, germlined M0064-F02, DX-2647, or DX-2655, and/or

the light chain immunoglobulin variable domain sequence comprises one or more (e.g., 1, 2, or 3) CDRs from the light chain of M0033-E05, M0063-F02, M0064-E04, M0064-F02, M0068-E03, M0070-H08, M0072-006, M0072-E03, M0072-G06, germlined M0064-E04, germlined M0064-F02, DX-2647, or DX-2655 (respectively), and the protein binds to and inhibits both IGF-II and IGF-IIE.

In some embodiments, the IGF-II/IGF-IIE binding protein is used in combination with a second therapeutic agent. In some embodiments, the second therapeutic agent is another IGF-II/IGF-IIE binding protein, e.g., another IGF-II/IGF-IIE binding protein described herein. In some embodiments, the second therapeutic agent is an anti-inflammatory drug (e.g., steroid), a cytotoxic drug, an immunosuppressive agent, a collagen synthesis inhibitor, or an endothelin receptor antagonist. For example, high doses of oral corticosteroids (e.g., prednisone, 40 to 80 mg daily) can be used. Cytotoxic drugs such as cyclophosphamide and immunosuppressants such as azathioprine (cyclophosphamide is also an immunosuppressant); collagen synthesis inhibitors such as Pirfenidone or endothelin receptor antagonists such as Bosentan can be used as the second agent. In preferred embodiments, the second agent is cyclophosphamide or azathioprine. In preferred embodiments, the second agent is cyclophosphamide in combination with a small dose of a steroid; epoprostenol; bosentan; or iloprost (e.g, aerolized iloprost). In some embodiments, the IGF-II/IGF-IIE binding protein is used in combination with surgery, e.g., lung transplantation. In some embodiments, the second agent is another treatment for SSc-associated pulmonary fibrosis such as anti-inflammatory drug e.g., a steroid (e.g., a corticosteroid (e.g., prednisone)), a cytotoxic drug (e.g., cyclophosphamide), an immunosuppressant (e.g., cyclophosphamide or azathioprine), a collagen synthesis inhibitor (e.g., Pirfenidone), or an endothelin receptor antagonist (e.g., Bosentan).

In some embodiments, the IGF-II/IGF-IIE binding protein decreases collagen and/or fibronectin production by greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% as compared to a standard, e.g., the subject's collagen and/or fibronectin production before the treatment.

The protein can bind to IGF-II and/or IGF-IIE, e.g., human IGF-II and/or IGF-IIE, with a binding affinity of at least 10⁵, 10⁶, 10⁷, 10⁸, 10⁹, 10¹⁰ and 10¹¹ M⁻¹. In one embodiment, the protein binds to human IGF-II and/or IGF-IIE with a K_{off} slower than 1×10⁻³, 5×10⁻⁴ s⁻¹, or 1×10⁻⁴ s⁻¹. In one embodiment, the protein binds to human IGF-II and/or IGF-IIE with a Kₒₙ faster than 1×10², 1×10³, or 5×10³ M⁻¹s⁻¹. In one embodiment, the protein inhibits both human IGF-II and human IGF-IIE activity, e.g., with a Ki of less than 10⁻⁵, 10⁻⁶, 10⁻⁷, 10⁻⁸, 10⁻⁹, and 10⁻¹⁰ M. In one embodiment, the protein inhibits either human IGF-II or human IGF-IIE activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. For example, the protein may modulate IGF-I receptor (IGF-1R) and/or an isoform of the insulin receptor (IR-A) activity, as well as IGF-II and/or IGF-IIE. The protein may inhibit IGF-1R, IR-A, and IGF-II and IGF-IIE activity. The affinity of the protein for human IGF-II and/or IGF-IIE can be characterized by a K_{D} of less than 100 nm, less than 10 nM, or less than 1 nM.

The protein can include one or more of the following characteristics: (a) a human CDR or human framework region; (b) the protein binds an epitope bound by a protein described herein, or an epitope that overlaps with such epitope; and (c) a primate CDR or primate framework region.

In a preferred embodiment, the protein is an antibody (e.g., a human antibody) having the light and heavy chains of antibodies selected from the group consisting of M0033-E05, M0063-F02, M0064-E04, M0064-F02, M0068-E03, M0070-H08, M0072-C06, M0072-E03, M0072-G06, germlined M0064-E04, germlined M0064-F02, DX-2647, and DX-2655.

In a preferred embodiment, the protein is an antibody (e.g., a human antibody) having the heavy chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the light chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having light and heavy antibody variable regions of an antibody selected from the group consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a heavy chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a light chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs selected from the corresponding CDRs of the group of heavy chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs and one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the light and heavy chains of DX-2647.

In a preferred embodiment, the protein is an antibody (e.g., a human antibody) having the heavy chain of DX-2647.

In a preferred embodiment, the protein is an antibody (e.g., a human antibody) having the light chain of DX-2647.

In a preferred embodiment, the protein is an antibody (e.g., a human antibody) having light and heavy antibody variable regions of an antibody selected from DX-2647.

In a preferred embodiment, the protein is an antibody (e.g., a human antibody) having a heavy chain antibody variable region of DX-2647.

In a preferred embodiment, the protein is an antibody (e.g., a human antibody) having a light chain antibody variable region of DX-2647.

In a preferred embodiment, the protein is an antibody (e.g., a human antibody) having one or more (e.g., 1, 2, or 3) heavy chain CDRs from the corresponding CDRs of the heavy chain of DX-2647.

In a preferred embodiment, the protein is an antibody (e.g., a human antibody) having one or more (e.g., 1, 2, or 3) light chain CDRs from the corresponding CDRs of the light chain of DX-2647.

In a preferred embodiment, the protein is an antibody (e.g., a human antibody) having one or more (e.g., 1, 2, or 3) heavy chain CDRs from the heavy chain of DX-2647 and one or more (e.g., 1, 2, or 3) light chain CDRs from the corresponding CDRs of the light chain of DX-2647.

In a preferred embodiment, the protein is an antibody (e.g., a human antibody) having the light and heavy chains of DX-2655.

In a preferred embodiment, the protein is an antibody (e.g., a human antibody) having the heavy chain of DX-2655.

In a preferred embodiment, the protein is an antibody (e.g., a human antibody) having the light chain of DX-2655.

In a preferred embodiment, the protein is an antibody (e.g., a human antibody) having light and heavy antibody variable regions of an antibody selected from DX-2655.

In a preferred embodiment, the protein is an antibody (e.g., a human antibody) having a heavy chain antibody variable region of DX-2655.

In a preferred embodiment, the protein is an antibody (e.g., a human antibody) having a light chain antibody variable region of DX-DX-2655.

In a preferred embodiment, the protein is an antibody (e.g., a human antibody) having one or more (e.g., 1, 2, or 3) heavy chain CDRs from the corresponding CDRs of the heavy chain of DX-2655.

In a preferred embodiment, the protein is an antibody (e.g., a human antibody) having one or more (e.g., 1, 2, or 3) light chain CDRs from the corresponding CDRs of the light chain of DX-2655.

In a preferred embodiment, the protein is an antibody (e.g., a human antibody) having one or more (e.g., 1, 2, or 3) heavy chain CDRs from the heavy chain of DX-2655 and one or more (e.g., 1, 2, or 3) light chain CDRs from the corresponding CDRs of the light chain of DX-2655.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In some embodiments, the affinity of the primate antibody for human IGF-II and/or IGF-IIE is characterized by a K_{D} of less than 1 nM.

In certain embodiments, the protein includes no sequences from mice or rabbits (e.g., is not a murine or rabbit antibody).

In certain embodiments, the protein is capable of binding to lung fibroblasts, or fibroblastic foci, e.g., that express IGF-II and/or IGF-IIE.

In one embodiment, protein is physically associated with a nanoparticle, and can be used to guide a nanoparticle to a cell expressing IGF-II and/or IGF-IIE on the cell surface.

In another aspect, the disclosure relates to a method of treating or preventing systemic sclerosis-associated pulmonary fibrosis in a subject, the method comprising:

administering to the subject an isolated protein (e.g., antibody, e.g., human antibody) comprising

(i) a heavy chain sequence and/or a light chain sequence, wherein the heavy chain sequence comprises the amino acid sequence of the heavy chain of M0080-G03 or M0073-C11, and/or the light chain sequence comprises the amino acid sequence of the light of M0080-G03 or M0073-C11 (respectively),

(ii) a heavy chain variable domain sequence and/or a light chain variable domain sequence, wherein the heavy chain variable domain sequence comprises the amino acid sequence of the heavy chain variable domain of M0080-G03 or M0073-C11, and/or the light chain variable domain sequence comprises the amino acid sequence of the light chain variable domain of M0080-G03 or M0073-C11 (respectively), or

(iii) one or more (e.g., 1, 2, or 3) heavy chain CDRs of antibody M0080-G03 or M0073-C11 and/or one or more (e.g., 1, 2, or 3) light chain CDRs selected from the corresponding CDRs of antibody M0080-G03 or M0073-C11 (respectively),

and the protein binds to and inhibits IGF-IIE but not IGF-II.

In some embodiments, the protein is used in combination with a second therapeutic agent. In some embodiments, the second therapeutic agent is another IGF-II/IGF-IIE binding protein, e.g., another IGF-II/IGF-IIE binding protein described herein. In some embodiments, the second therapeutic agent is an anti-inflammatory drug (e.g., steroid), a cytotoxic drug, an immunosuppressive agent, a collagen synthesis inhibitor, or an endothelin receptor antagonist. For example, high doses of oral corticosteroids (e.g., prednisone, 40 to 80 mg daily) can be used. Cytotoxic drugs such as cyclophosphamide and immunosuppressants such as azathioprine (cyclophosphamide is also an immunosuppressant); collagen synthesis inhibitors such as Pirfenidone or endothelin receptor antagonists such as Bosentan can be used as the second agent. In preferred embodiments, the second agent is cyclophosphamide or azathioprine. In preferred embodiments, the second agent is cyclophosphamide in combination with a small dose of a steroid; epoprostenol; bosentan; or iloprost (e.g, aerolized iloprost). In some embodiments, the protein is used in combination with surgery, e.g., lung transplantation. In some embodiments, the second agent is another treatment for SSc-associated pulmonary fibrosis such as anti-inflammatory drug e.g., a steroid (e.g., a corticosteroid (e.g., prednisone)), a cytotoxic drug (e.g., cyclophosphamide), an immunosuppressant (e.g., cyclophosphamide or azathioprine), a collagen synthesis inhibitor (e.g., Pirfenidone), or an endothelin receptor antagonist (e.g., Bosentan).

In some embodiments, the protein decreases collagen and/or fibronectin production by greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% as compared to a standard, e.g., the subject's collagen and/or fibronectin production before the treatment.

The protein can bind to IGF-IIE, e.g., human IGF-IIE, with a binding affinity of at least 10⁵, 10⁶, 10⁷, 10⁸, 10⁹, 10¹⁰ and 10¹¹ M⁻¹. In one embodiment, the protein binds to human IGF-IIE with a K_{off} slower than 1×10⁻³, 5×10⁴ s⁻¹, or 1×10⁴ s⁻¹. In one embodiment, the protein binds to human IGF-IIE with a Kₒₙ faster than 1×10², 1×10³, or 5×10³ M⁻¹ s⁻¹. In one embodiment, the protein inhibits human IGF-IIE activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. For example, the protein may modulate IGF-I receptor (IGF-1R) and/or an isoform of the insulin receptor (IR-A) activity, as well as IGF-IIE. The protein may inhibit IGF-1R, IR-A, and IGF-IIE activity. The affinity of the protein for human IGF-IIE can be characterized by a K_{D} of less than 100 nm, less than 10 nM, or less than 1 nM.

The protein can include one or more of the following characteristics: (a) a human CDR or human framework region; (b) the protein binds an epitope bound by a protein described herein, or an epitope that overlaps with such epitope; and (c) a primate CDR or primate framework region.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In some embodiments, the affinity of the primate antibody for human IGF-IIE is characterized by a K_{D} of less than 1 nM.

In certain embodiments, the protein includes no sequences from mice or rabbits (e.g., is not a murine or rabbit antibody).

In certain embodiments, the protein is capable of binding to lung fibroblasts, or fibroblastic foci, e.g., that express IGF-IIE.

In one embodiment, the protein is physically associated with a nanoparticle, and can be used to guide a nanoparticle to a cell expressing IGF-IIE on the cell surface.

In some aspects, the disclosure features an isolated protein (e.g., antibody, e.g., human antibody) comprising

(i) a heavy chain sequence and/or a light chain sequence, wherein the heavy chain sequence comprises the amino acid sequence of the heavy chain of M0080-G03 or M0073-C11, and/or the light chain sequence comprises the amino acid sequence of the light of M0080-G03 or M0073-C11 (respectively),

(ii) a heavy chain variable domain sequence and/or a light chain variable domain sequence, wherein the heavy chain variable domain sequence comprises the amino acid sequence of the heavy chain variable domain of M0080-G03 or M0073-C11, and/or the light chain variable domain sequence comprises the amino acid sequence of the light chain variable domain of M0080-G03 or M0073-C11 (respectively), or

(iii) one or more (e.g., 1, 2, or 3) heavy chain CDRs of antibody M0080-G03 or M0073-C11 and/or one or more (e.g., 1, 2, or 3) light chain CDRs selected from the corresponding CDRs of antibody M0080-G03 or M0073-C11 (respectively).

In some embodiments, the protein binds to and inhibits IGF-IIE but not IGF-II.

The protein can bind to IGF-IIE, e.g., human IGF-IIE, with a binding affinity of at least 10⁵, 10⁶, 10⁷, 10⁸, 10⁹, 10¹⁰ and 10¹¹ M⁻¹. In one embodiment, the protein binds to human IGF-IIE with a K_{off} slower than 1×10⁻³, 5×10⁻⁴ s⁻¹, or 1×10⁻⁴ s⁻¹. In one embodiment, the protein binds to human IGF-IIE with a Kₒₙ faster than 1×10², 1×10³, or 5×10³ M⁻¹ s⁻¹. In one embodiment, the protein inhibits human IGF-IIE activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. For example, the protein may modulate IGF-I receptor (IGF-1R) and/or an isoform of the insulin receptor (IR-A) activity, as well as IGF-IIE. The protein may inhibit IGF-1R, IR-A, and IGF-IIE activity. The affinity of the protein for human IGF-IIE can be characterized by a K_{D} of less than 100 nm, less than 10 nM, or less than 1 nM.

The protein can include one or more of the following characteristics: (a) a human CDR or human framework region; (b) the protein binds an epitope bound by a protein described herein, or an epitope that overlaps with such epitope; and (c) a primate CDR or primate framework region.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate to antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In some embodiments, the affinity of the primate antibody for human IGF-IIE is characterized by a K_{D} of less than 1 nM.

In certain embodiments, the protein includes no sequences from mice or rabbits (e.g., is not a murine or rabbit antibody).

In certain embodiments, the protein is capable of binding to lung fibroblasts, or fibroblastic foci, e.g., that express IGF-IIE.

In one embodiment, the protein is physically associated with a nanoparticle, and can be used to guide a nanoparticle to a cell expressing IGF-IIE on the cell surface.

In other aspects, the disclosure relates to a method of treating or preventing systemic sclerosis-associated pulmonary fibrosis in a subject, the method comprising:
administering to the subject an isolated protein capable of specifically binding to the following consensus sequence or a functional fragment thereof:

TXCGGXLVXXLXXXXXXXXFXXXXPXXRVXRXSRGXVEEXCFRXXXXXXX
XXY

wherein X is any amino acid.

In some embodiments, the protein is capable of specifically binding to the following consensus sequence or a functional fragment thereof:

SETLCGGELVDTLQFVCGDRGFYFSRPASRVSRRSRGIVEECCFRSCDLALLETYCATPA.

In some embodiments, the protein is used in combination with a second therapeutic agent. In some embodiments, the second therapeutic agent is another IGF-II/IGF-IIE binding protein, e.g., another IGF-II/IGF-IIE binding protein described herein. In some embodiments, the second therapeutic agent is an anti-inflammatory drug (e.g., steroid), a cytotoxic drug, an immunosuppressive agent, a collagen synthesis inhibitor, or an endothelin receptor antagonist. For example, high doses of oral corticosteroids (e.g., prednisone, 40 to 80 mg daily) can be used. Cytotoxic drugs such as cyclophosphamide and immunosuppressants such as azathioprine (cyclophosphamide is also an immunosuppressant); collagen synthesis inhibitors such as Pirfenidone or endothelin receptor antagonists such as Bosentan can be used as the second agent. In preferred embodiments, the second agent is cyclophosphamide or azathioprine. In preferred embodiments, the second agent is cyclophosphamide in combination with a small dose of a steroid; epoprostenol; bosentan; or iloprost (e.g, aerolized iloprost). In some embodiments, the protein is used in combination with surgery, e.g., lung transplantation. In some embodiments, the second agent is another treatment for SSc-associated pulmonary fibrosis such as anti-inflammatory drug e.g., a steroid (e.g., a corticosteroid (e.g., prednisone)), a cytotoxic drug (e.g., cyclophosphamide), an immunosuppressant (e.g., cyclophosphamide or azathioprine), a collagen synthesis inhibitor (e.g., Pirfenidone), or an endothelin receptor antagonist (e.g., Bosentan).

In some embodiments, the protein decreases collagen and/or fibronectin production by greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% as compared to a standard, e.g., the subject's collagen and/or fibronectin production before the treatment.

The protein can include one or more of the following characteristics: (a) a human CDR or human framework region; (b) the protein binds an epitope bound by a protein described herein, or an epitope that overlaps with such epitope; and (c) a primate CDR or primate framework region.

The protein can bind to IGF-II and/or IGF-IIE, e.g., human IGF-II and/or IGF-IIE, with a binding affinity of at least 10⁵, 10⁶, 10⁷, 10⁸, 10⁹, 10¹⁰ and 10¹¹ M⁻¹. In one embodiment, the protein binds to human IGF-II and/or IGF-IIE with a K_{off} slower than 1×10⁻³, 5×10⁻⁴ s⁻¹, or 1×10⁻⁴ s⁻¹. In one embodiment, the protein binds to human IGF-II and/or IGF-IIE with a Kₒₙ faster than 1×10², 1×10³, or 5×10³ M⁻¹s⁻¹. In one embodiment, the protein inhibits both human IGF-II and human IGF-IIE activity, e.g., with a Ki of less than 10⁻⁵, 10⁻⁶, 10⁻⁷, 10⁻⁸, 10⁻⁹, and 10⁻¹⁰ M. In one embodiment, the protein inhibits either human IGF-II or human IGF-IIE activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. For example, the protein may modulate IGF-I receptor (IGF-1R) and/or an isoform of the insulin receptor (IR-A) activity, as well as IGF-II and/or IGF-IIE. The protein may inhibit IGF-1R, IR-A, and IGF-II and IGF-IIE activity. The affinity of the protein for human IGF-II and/or IGF-IIE can be characterized by a K_{D} of less than 100 nm, less than 10 nM, or less than 1 nM.

In a preferred embodiment, the protein is an antibody (e.g., a human antibody) having the light and heavy chains of antibodies selected from the group consisting of M0033-E05, M0063-F02, M0064-E04, M0064-F02, M0068-E03, M0070-H08, M0072-C06, M0072-E03, M0072-G06, germlined M0064-E04, germlined M0064-F02, DX-2647, and DX-2655.

In a preferred embodiment, the protein is an antibody (e.g., a human antibody) having the heavy chain of an antibody selected from the group consisting of: M0033-E05, M0063-F02, M0064-E04, M0064-F02, M0068-E03, M0070-H08, M0072-006, M0072-E03, M0072-G06, germlined M0064-E04, and germlined M0064-F02.

In a preferred embodiment, the protein is an antibody (e.g., a human antibody) having the light chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having light and heavy antibody variable regions of an antibody selected from the group consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a heavy chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a light chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs selected from the corresponding CDRs of the group of heavy chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs and one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the light and heavy chains of DX-2647.

In a preferred embodiment, the protein is an antibody (e.g., a human antibody) having the heavy chain of DX-2647.

In a preferred embodiment, the protein is an antibody (e.g., a human antibody) having the light chain of DX-2647.

In a preferred embodiment, the protein is an antibody (e.g., a human antibody) having light and heavy antibody variable regions of an antibody selected from DX-2647.

In a preferred embodiment, the protein is an antibody (e.g., a human antibody) having a heavy chain antibody variable region of DX-2647.

In a preferred embodiment, the protein is an antibody (e.g., a human antibody) having a light chain antibody variable region of DX-2647.

In a preferred embodiment, the protein is an antibody (e.g., a human antibody) having one or more (e.g., 1, 2, or 3) heavy chain CDRs from the corresponding CDRs of the heavy chain of DX-2647.

In a preferred embodiment, the protein is an antibody (e.g., a human antibody) having one or more (e.g., 1, 2, or 3) light chain CDRs from the corresponding CDRs of the light chain of DX-2647.

In a preferred embodiment, the protein is an antibody (e.g., a human antibody) having one or more (e.g., 1, 2, or 3) heavy chain CDRs from the heavy chain of DX-2647 and one or more (e.g., 1, 2, or 3) light chain CDRs from the corresponding CDRs of the light chain of DX-2647.

In a preferred embodiment, the protein is an antibody (e.g., a human antibody) having the light and heavy chains of DX-2655.

In a preferred embodiment, the protein is an antibody (e.g., a human antibody) having the heavy chain of DX-2655.

In a preferred embodiment, the protein is an antibody (e.g., a human antibody) having the light chain of DX-2655.

In a preferred embodiment, the protein is an antibody (e.g., a human antibody) having light and heavy antibody variable regions of an antibody selected from DX-2655.

In a preferred embodiment, the protein is an antibody (e.g., a human antibody) having a heavy chain antibody variable region of DX-2655.

In a preferred embodiment, the protein is an antibody (e.g., a human antibody) having a light chain antibody variable region of DX-DX-2655.

In a preferred embodiment, the protein is an antibody (e.g., a human antibody) having one or more (e.g., 1, 2, or 3) heavy chain CDRs from the corresponding CDRs of the heavy chain of DX-2655.

In a preferred embodiment, the protein is an antibody (e.g., a human antibody) having one or more (e.g., 1, 2, or 3) light chain CDRs from the corresponding CDRs of the light chain of DX-2655.

In a preferred embodiment, the protein is an antibody (e.g., a human antibody) having one or more (e.g., 1, 2, or 3) heavy chain CDRs from the heavy chain of DX-2655 and one or more (e.g., 1, 2, or 3) light chain CDRs from the corresponding CDRs of the light chain of DX-2655.

In one embodiment, the protein has HC and LC variable domain sequences. In some embodiments, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In some embodiments, the affinity of the primate antibody for human IGF-II and/or IGF-IIE is characterized by a K_{D} of less than 1 nM.

In certain embodiments, the protein includes no sequences from mice or rabbits (e.g., is not a murine or rabbit antibody).

In certain embodiments, the protein is capable of binding to lung fibroblasts, or fibroblastic foci, e.g., that express IGF-II and/or IGF-IIE.

In one embodiment, protein is physically associated with a nanoparticle, and can be used to guide a nanoparticle to a cell expressing IGF-II and/or IGF-IIE on the cell surface.

In some aspects, the disclosure provides methods of using proteins that bind to both or either of IGF-II and IGF-IIE, herein referred to as “IGF-II/IGF-IIE binding proteins,” for the treatment and/or prevention of systemic sclerosis-associated pulmonary fibrosis. These proteins include antibodies and antibody fragments (e.g., primate antibodies and Fabs, especially human antibodies and Fabs) that bind to and/or inhibit both IGF-II and IGF-IIE consequential binding, e.g., binding to IGF-1R and/or an isoform of the insulin receptor (IR-A). The IGF-II/IGF-IIE binding proteins can be used in the treatment of diseases, particularly human disease, such as systemic sclerosis-associated pulmonary fibrosis, in which excess or inappropriate activity of IGF-II and/or IGF-IIE features. In many cases, the proteins have tolerable low or no toxicity.

In one aspect, the disclosure features methods for the treatment and/or prevention of systemic sclerosis-associated pulmonary fibrosis that utilize a protein (e.g., an isolated protein) that binds to IGF-II and/or IGF-IIE (e.g., human IGF-II and/or IGF-IIE) and includes at least one immunoglobulin variable region. For example, the protein includes a heavy chain (HC) immunoglobulin variable domain sequence and a light chain (LC) immunoglobulin variable domain sequence. In one embodiment, the protein binds to and inhibits IGF-II and/or IGF-IIE (e.g., human IGF-II and/or IGF-IIE) consequential binding, e.g., binding to IGF-1R and/or an isoform of the insulin receptor (IR-A). In another embodiment, the protein binds to and/or inhibits only IGF IIE but not IGF-II consequential binding.

The protein can include one or more of the following characteristics: (a) a human CDR or human framework region; (b) the HC immunoglobulin variable domain sequence comprises one or more CDRs that are at least 85, 88, 90, 92, 94, 95, 96, 97, 98, 99, or 100% identical to a CDR of a HC variable domain described herein; (c) the LC immunoglobulin variable domain sequence comprises one or more CDRs that are at least 85, 88, 90, 92, 94, 95, 96, 97, 98, 99, or 100% identical to a CDR of a LC variable domain described herein; (d) the LC immunoglobulin variable domain sequence is at least 85, 88, 90, 92, 94, 95, 96, 97, 98, 99, or 100% identical to a LC variable domain described herein; (e) the HC immunoglobulin variable domain sequence is at least 85, 88, 90, 92, 94, 95, 96, 97, 98, 99, or 100% identical to a HC variable domain described herein; (f) the protein binds an epitope bound by a protein described herein, or an epitope that overlaps with such epitope; and (g) a primate CDR or primate framework region.

The protein can bind to IGF-II and/or IGF-IIE, e.g., human IGF-II and/or IGF-IIE, with a binding affinity of at least 10⁵, 10⁶, 10⁷, 10⁸, 10⁹, 10¹⁰ and 10¹¹ M⁻¹. In one embodiment, the protein binds to human IGF-II and/or IGF-IIE with a K_{off} slower than 1×10⁻³, 5×10⁻⁴ s⁻¹, or 1×10⁻⁴ s⁻¹. In one embodiment, the protein binds to human IGF-II and/or IGF-IIE with a Kₒₙ faster than 1×10², 1×10³, or 5×10³ M⁻¹ s⁻¹. In one embodiment, the protein inhibits both human IGF-II and human IGF-IIE activity, e.g., with a Ki of less than 10⁻⁵, 10⁻⁶, 10⁻⁷, 10⁻⁸, 10⁻⁹, and 10⁻¹⁰ M. In one embodiment, the protein inhibits either human IGF-II or human IGF-IIE activity, e.g., with a Ki of less than 10⁻⁵, 10⁻⁶, 10⁻⁷, 10⁻⁸, 10⁻⁹, and 10⁻¹⁰M. The protein can have, for example, an IC50 of less than 100 nM, 10 nM or 1 nM. For example, the protein may modulate IGF-I receptor (IGF-1R) and/or an isoform of the insulin receptor (IR-A) activity, as well as IGF-II and/or IGF-IIE. The protein may inhibit IGF-1R, IR-A, and IGF-II and IGF-IIE activity. The affinity of the protein for human IGF-II and/or IGF-IIE can be characterized by a K_{D} of less than 100 nm, less than 10 nM, or less than 1 nM.

In a preferred embodiment, the protein is an antibody (e.g., a human antibody) having the light and heavy chains of antibodies selected from the group consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the heavy chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the light chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having light and heavy antibody variable regions of an antibody selected from the group consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a heavy chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a light chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs selected from the corresponding CDRs of the group of heavy chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs and one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the light and heavy chains of DX-2647.

In a preferred embodiment, the protein is an antibody (e.g., a human antibody) having the heavy chain of DX-2647.

In a preferred embodiment, the protein is an antibody (e.g., a human antibody) having the light chain of DX-2647.

In a preferred embodiment, the protein is an antibody (e.g., a human antibody) having light and heavy antibody variable regions of an antibody selected from DX-2647.

In a preferred embodiment, the protein is an antibody (e.g., a human antibody) having a heavy chain antibody variable region of DX-2647.

In a preferred embodiment, the protein is an antibody (e.g., a human antibody) having a light chain antibody variable region of DX-2647.

In a preferred embodiment, the protein is an antibody (e.g., a human antibody) having one or more (e.g., 1, 2, or 3) heavy chain CDRs from the corresponding CDRs of the heavy chain of DX-2647.

In a preferred embodiment, the protein is an antibody (e.g., a human antibody) having one or more (e.g., 1, 2, or 3) light chain CDRs from the corresponding CDRs of the light chain of DX-2647.

In a preferred embodiment, the protein is an antibody (e.g., a human antibody) having one or more (e.g., 1, 2, or 3) heavy chain CDRs from the heavy chain of DX-2647 and one or more (e.g., 1, 2, or 3) light chain CDRs from the corresponding CDRs of the light chain of DX-2647.

In a preferred embodiment, the protein is an antibody (e.g., a human antibody) having the light and heavy chains of DX-2655.

In a preferred embodiment, the protein is an antibody (e.g., a human antibody) having the heavy chain of DX-2655.

In a preferred embodiment, the protein is an antibody (e.g., a human antibody) having the light chain of DX-2655.

In a preferred embodiment, the protein is an antibody (e.g., a human antibody) having light and heavy antibody variable regions of an antibody selected from DX-2655.

In a preferred embodiment, the protein is an antibody (e.g., a human antibody) having a heavy chain antibody variable region of DX-2655.

In a preferred embodiment, the protein is an antibody (e.g., a human antibody) having a light chain antibody variable region of DX-DX-2655.

In a preferred embodiment, the protein is an antibody (e.g., a human antibody) having one or more (e.g., 1, 2, or 3) heavy chain CDRs from the corresponding CDRs of the heavy chain of DX-2655.

In a preferred embodiment, the protein is an antibody (e.g., a human antibody) having one or more (e.g., 1, 2, or 3) light chain CDRs from the corresponding CDRs of the light chain of DX-2655.

In a preferred embodiment, the protein is an antibody (e.g., a human antibody) having one or more (e.g., 1, 2, or 3) heavy chain CDRs from the heavy chain of DX-2655 and one or more (e.g., 1, 2, or 3) light chain CDRs from the corresponding CDRs of the light chain of DX-2655.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In some embodiments, the affinity of the primate antibody for human IGF-II and/or IGF-IIE is characterized by a K_{D} of less than 1 nM.

In certain embodiments, the protein includes no sequences from mice or rabbits (e.g., is not a murine or rabbit antibody).

In certain embodiments, the protein may be capable of binding to lung fibroblasts, or fibroblastic foci, e.g., that express IGF-II and/or IGF-IIE.

In one embodiment, protein is physically associated with a nanoparticle, and can be used to guide a nanoparticle to a cell expressing IGF-II and/or IGF-IIE on the cell surface.

A binding protein described herein can be provided as a pharmaceutical composition, e.g., including a pharmaceutically acceptable carrier. The composition can be at least 10, 20, 30, 50, 75, 85, 90, 95, 98, 99, or 99.9% free of other protein species. In some embodiments, the binding protein can be produced under GMP (good manufacturing practices). In some embodiments, the binding protein is provided in pharmaceutically acceptable carriers, e.g., suitable buffers or excipients.

In another aspect, the disclosure features a method of detecting IGF-II and/or IGF-IIE in a sample. The method includes: contacting the sample with an IGF-II/IGF-IIE binding protein (e.g., an IGF-II/IGF-IIE binding protein described herein); and detecting an interaction between the protein and the IGF-II and/or IGF-IIE, if present. In some embodiments, the protein includes a detectable label. An IGF-II/IGF-IIE binding protein can be used to detect IGF-II and/or IGF-IIE in a subject. The method includes: administering an IGF-II/IGF-IIE binding protein (e.g., an IGF-II/IGF-IIE binding protein described herein) to a subject; and detecting an interaction between the protein and the IGF-II and/or IGF-IIE in the subject, if present. In some embodiments, the protein further includes a detectable label. For example, the detecting comprises imaging the subject.

In another aspect, the disclosure features a method of modulating IGF-II and/or IGF-IIE activity, e.g., in a method of treating or preventing systemic sclerosis-associated pulmonary fibrosis. The method includes: contacting IGF-II and/or IGF-IIE with an IGF-II/IGF-IIE binding protein (e.g., in a human subject), thereby modulating IGF-II and/or IGF-IIE activity.

In another aspect, the disclosure features a method of treating SSc-associated pulmonary fibrosis. The method includes: administering, to a subject, an IGF-II/IGF-IIE binding protein in an amount sufficient to treat SSc-associated pulmonary fibrosis in the subject. The method can further include providing to the subject a second therapy that is therapy for SSc-associated pulmonary fibrosis, e.g., an anti-inflammatory agent, e.g., a steroid, e.g., a corticosteroid (e.g., prednisone)), a cytotoxic drug (e.g., cyclophosphamide), an immunosuppressant (e.g., cyclophosphamide or azathioprine), a collagen synthesis inhibitor (e.g., Pirfenidone), an endothelin receptor antagonist (e.g., Bosentan (e.g., TRACLEER®)) or surgery (e.g., a lung transplant).

IGF-II/IGF-IIE binding proteins are useful for targeted delivery of an agent to a subject (e.g., a subject who has or is suspected of having a tumor), e.g., to direct the agent to the lung (e.g., lung fibroblasts or fibroblastic foci) in the subject. For example, an IGF-II/IGF-IIE binding protein (e.g., an IGF-II/IGF-IIE binding protein described herein) that is coupled to an agent (such as a toxin, drug, or a radionuclide (e.g., ¹³¹I, ⁹⁰Y, ¹⁷⁷Lu)) can be administered to a subject who has or is suspected of having SSc-associated pulmonary fibrosis.

In another aspect, the disclosure features a method of imaging a subject. The method includes administering an IGF-II/IGF-IIE binding protein (e.g., an IGF-II/IGF-IIE binding protein described herein) to the subject. In some embodiments, the protein is one that does not substantially inhibit IGF-II or IGF-IIE activity. In some embodiments, the protein is one that substantially inhibits IGF-II or IGF-IIE activity. The IGF-II/IGF-IIE binding protein may include a detectable label (e.g., a radionuclide or an MRI-detectable label). In one embodiment, the subject has or is suspected of having SSc-associated pulmonary fibrosis. The method is useful for SSc-associated pulmonary fibrosis diagnosis.

In one aspect, the disclosure features the use of an IGF-II/IGF-IIE binding protein described herein for the manufacture of a medicament for the treatment of a disorder described herein, e.g., SSc-associated pulmonary fibrosis.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1(A) and 1(B) show polypeptide folds as determined by the crystallographic analysis of a complex of IGF-II with M0064-F02 Fab (as described in Example 8 below). Helices are indicated by curled ribbons and beta sheets by broad arrows.

FIGS. 2(A) and 2(B) give typical profiles obtained from SPR affinity measurements of one of the antibodies interacting with the Binding Proteins BP2 and BP4. (A) data for M0063-F02, (B) data for M0064-E04 candidate antibody.

### DETAILED DESCRIPTION

### Definitions

For convenience, before further description of the present invention, certain terms employed in the specification, examples and appended claims are defined here. Other terms are defined as they appear in the specification.

Herein, the construct comprising amino acids 1-104 of the IGF-II precursor protein, which encompass the E domain, is referred to as “IGF-IIE”.

The singular forms “a”, “an”, and “the” include plural references unless the context clearly dictates otherwise.

The term “agonist”, as used herein, is meant to refer to an agent that mimics or up-regulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

“Antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide or enzyme substrate. An antagonist can also be a compound that downregulates expression of a gene or which reduces the amount of expressed protein present.

The term “antibody” refers to a protein that includes at least one immunoglobulin variable domain (variable region) or immunoglobulin variable domain (variable region) sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J. Immunol. 1996; 26(3):629-39.)) as well as complete antibodies. An antibody can have the structural features of IgA, IgG, IgE, IgD, IgM (as well as subtypes thereof). Antibodies may be from any source, but primate (human and non-human primate) and primatized are preferred.

The VH and VL regions can be further subdivided into regions of hypervariability, termed “complementarity determining regions” (“CDRs”), interspersed with regions that are more conserved, termed “framework regions” (“FRs”). The extent of the framework region and CDRs have been defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain such that one or more CDR regions are positioned in a conformation suitable for an antigen binding site. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form an antigen binding site, e.g., a structure that preferentially interacts with IGF-II and/or IGF-IIE.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of types kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883.

Antibody fragments can be obtained using any appropriate technique including conventional techniques known to those with skill in the art. The term “monospecific antibody” refers to an antibody that displays a single binding specificity and affinity for a particular target, e.g., epitope. This term includes a “monoclonal antibody” or “monoclonal antibody composition,” which as used herein refers to a preparation of antibodies or fragments thereof of single molecular composition, irrespective of how the antibody was generated.

As used herein, “binding affinity” refers to the apparent association constant or K_{A}. The K_{A} is the reciprocal of the dissociation constant (K_{D}). A binding protein may, for example, have a binding affinity of at least 10⁵, 10⁶, 10⁷,10⁸, 10⁹, 10¹⁰ and 10¹¹ M⁻¹ for a particular target molecule, e.g., IGF-II and/or IGF-IIE. Higher affinity binding of a binding protein to a first target relative to a second target can be indicated by a higher K_{A} (or a smaller numerical value K_{D}) for binding the first target than the K_{A} (or numerical value K_{D}) for binding the second target. In such cases, the binding protein has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N·[Free]/((1/K_{A})+[Free]).

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The term “binding protein” refers to a protein that can interact with a target molecule. This term is used interchangeably with “ligand.” An “IGF-II/IGF-IIE binding protein” refers to a protein that can interact with both IGF-II and IGF-IIE, and includes, in particular, proteins that preferentially interact with and/or inhibit both IGF-II and IGF-IIE. For example, the IGF-II/IGF-IIE binding protein is an antibody. Likewise, an “IGF-IIE binding protein” refers to a protein that can interact with only IGF-IIE, and includes, in particular, proteins that preferentially interact with and/or inhibit only IGF-IIE.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). It is possible for many framework and CDR amino acid residues to include one or more conservative substitutions. An IGF-II/IGF-IIE binding protein may have mutations (e.g., at least one, two, or four, and/or less than 15, 10, 5, or 3) relative to a binding protein described herein (e.g., a conservative or non-essential amino acid substitutions), which do not have a substantial effect on protein function. Whether or not a particular substitution will be tolerated, i.e., will not adversely affect biological properties, such as binding activity can be predicted, e.g., by evaluating whether the mutation is conservative or by the method of Bowie, et al. (1990) Science 247:1306-1310.

Motif sequences for biopolymers can include positions which can be varied amino acids. For example, the symbol “X” in such a context generally refers to any amino acid (e.g., any of the twenty natural amino acids or any of the nineteen non-cysteine amino acids). Other allowed amino acids can also be indicated for example, using parentheses and slashes. For example, “(A/W/F/N/Q)” means that alanine, tryptophan, phenylalanine, asparagine, and glutamine are allowed at that particular position.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

An “epitope” refers to the site on a target compound that is bound by a binding protein (e.g., an antibody such as a Fab or full length antibody). In the case where the target compound is a protein, the site can be entirely composed of amino acid components, entirely composed of chemical modifications of amino acids of the protein (e.g., glycosyl moieties), or composed of combinations thereof. Overlapping epitopes include at least one common amino acid residue, glycosyl group, phosphate group, sulfate group, or other molecular feature.

An (first) antibody “binds to the same epitope” as another (second) antibody if the antibody binds to the same site on a target compound that the second antibody binds, or binds to a site that overlaps (e.g., 50%, 60%, 70%, 80%, 90%, or 100% overlap, e.g., in terms of amino acid sequence or other molecular feature (e.g., glycosyl group, phosphate group, or sulfate group) with the site that the second antibody binds.

An (first) antibody “competes for binding” with another (second) antibody if the binding of the first antibody to its epitope decreases (e.g., by 10%, 20%, 30%, 40%, 50%, 60%, 70%, 80%, 90%, 100%, or more) the amount of the second antibody that binds to its epitope. The competition can be direct (e.g., the first antibody binds to an epitope that is the same as, or overlaps with, the epitope bound by the second antibody), or indirect (e.g., the binding of the first antibody to its epitope causes a steric change in the target compound that decreases the ability of the second antibody to bind to its epitope).

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

In a preferred embodiment, the length of a reference sequence aligned for comparison purposes is at least 30%, preferably at least 40%, more preferably at least 50%, even more preferably at least 60%, and even more preferably at least 70%, 80%, 90%, 92%, 95%, 97%, 98%, or 100% of the length of the reference sequence. For example, the reference sequence may be the length of the immunoglobulin variable domain sequence.

A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified. The disclosure includes nucleic acids that hybridize with low, medium, high, or very high stringency to a nucleic acid described herein or to a complement thereof, e.g., nucleic acids encoding a binding protein described herein. The nucleic acids can be the same length or within 30, 20, or 10% of the length of the reference nucleic acid. The nucleic acid can correspond to a region encoding an immunoglobulin variable domain sequence described herein.

An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 92, 95, 98, or 99% pure on a weight-weight basis.

The term “modulator” refers to a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

A “non-essential” amino acid residue is a residue that can be altered from the wild-type sequence of the binding agent, e.g., the antibody, without abolishing or more preferably, without substantially altering a biological activity, whereas changing an “essential” amino acid residue results in a substantial loss of activity.

A “patient”, “subject” or “host” to be treated by the subject method may mean either a human or non-human animal.

The term “preventing” or to “prevent” a disease in a subject refers to subjecting the subject to a pharmaceutical treatment, e.g., the administration of a drug, such that at least one symptom of the disease is prevented, that is, administered prior to clinical manifestation of the unwanted condition (e.g., disease or other unwanted state of the host animal) so that it protects the host against developing the unwanted condition. “Preventing” a disease may also be referred to as “prophylaxis” or “prophylactic treatment.”

As used herein, the term “substantially identical” (or “substantially homologous”) is used herein to refer to a first amino acid or nucleic acid sequence that contains a sufficient number of identical or equivalent (e.g., with a similar side chain, e.g., conserved amino acid substitutions) amino acid residues or nucleotides to a second amino acid or nucleic acid sequence such that the first and second amino acid or nucleic acid sequences have (or encode proteins having) similar activities, e.g., a binding activity, a binding preference, or a biological activity. In the case of antibodies, the second antibody has the same specificity and has at least 50%, at least 25%, or at least 10% of the affinity relative to the same antigen.

Sequences similar or homologous (e.g., at least about 85% sequence identity) to the sequences disclosed herein are also part of this application. In some embodiments, the sequence identity can be about 85%, 90%, 91%, 92%, 93%, 94%, 95%, 96%, 97%, 98%, 99% or higher. In addition, substantial identity exists when the nucleic acid segments hybridize under selective hybridization conditions (e.g., highly stringent hybridization conditions), to the complement of the strand. The nucleic acids may be present in whole cells, in a cell lysate, or in a partially purified or substantially pure form.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular binding proteins may show a difference, e.g., in specificity or binding, that are statistically significant (e.g., P value<0.05 or 0.02). The terms “induce”, “inhibit”, “potentiate”, “elevate”, “increase”, “decrease” or the like, e.g., which denote distinguishable qualitative or quantitative differences between two states, and may refer to a difference, e.g., a statistically significant difference, between the two states.

The term “treat” or “treatment” refers to the application or administration of an agent, alone or in combination with one or more other agents (e.g., a second agent) to a subject, e.g., a patient, e.g., a patient who has a disorder (e.g., a disorder as described herein), a symptom of a disorder or a predisposition for a disorder, e.g., to cure, heal, alleviate, relieve, alter, remedy, ameliorate, improve or affect the disorder, the symptoms of the disorder or the predisposition toward the disorder. Treating a cell refers to a reduction in an activity of a cell, e.g., ability of an endothelial cell to form tubes or vessels. A reduction does not necessarily require a total elimination of activity, but a reduction, e.g., a statistically significant reduction, in the activity or the number of cells.

### IGF-II/IGF-IIE Binding Proteins

The disclosure provides the use of proteins (e.g., binding proteins) that bind to both or either IGF-II and/or IGF-IIE (e.g., human IGF-II and/or IGF-IIE) and include at least one immunoglobin variable region in methods for treating (or preventing) SSc-associated pulmonary fibrosis. For example, the IGF-II/IGF-IIE binding protein includes a heavy chain (HC) immunoglobulin variable domain sequence and a light chain (LC) immunoglobulin variable domain sequence. A number of exemplary IGF-II/IGF-IIE and IGF-IIE binding proteins are described herein.

The IGF-II/IGF-IIE binding protein may be an isolated protein (e.g., at least 70, 80, 90, 95, or 99% free of other proteins).

The IGF-II/IGF-IIE binding protein may additionally inhibit both IGF-II and IGF-IIE, e.g., human IGF-II and IGF-IIE.

In one aspect, the disclosure features a protein (e.g., an isolated protein) that binds to IGF-II and IGF-IIE (e.g., human IGF-II and IGF-IIE) and includes at least one immunoglobulin variable region. For example, the protein includes a heavy chain (HC) immunoglobulin variable domain sequence and/or a light chain (LC) immunoglobulin variable domain sequence. In one embodiment, the protein binds to and inhibits IGF-II and IGF-IIE, e.g., human IGF-II and/or IGF-IIE.

The protein can include one or more of the following characteristics: (a) a human CDR or human framework region; (b) the HC immunoglobulin variable domain sequence comprises one or more (e.g., 1, 2, or 3) CDRs that are at least 85, 88, 90, 92, 94, 95, 96, 97, 98, 99, or 100% identical to a CDR of a HC variable domain described herein; (c) the LC immunoglobulin variable domain sequence comprises one or more (e.g., 1, 2, or 3) CDRs that are at least 85, 88, 90, 92, 94, 95, 96, 97, 98, 99, or 100% identical to a CDR of a LC variable domain described herein; (d) the LC immunoglobulin variable domain sequence is at least 85, 88, 90, 92, 94, 95, 96, 97, 98, 99, or 100% identical to a LC variable domain described herein; (e) the HC immunoglobulin variable domain sequence is at least 85, 88, 90, 92, 94, 95, 96, 97, 98, 99, or 100% identical to a HC variable domain described herein; (f) the protein binds an epitope bound by a protein described herein, or an epitope that overlaps with such epitope; and (g) a primate CDR or primate framework region.

In certain embodiments, the protein binds the following epitope of IGF-II, or a fragment thereof:

TXCGGXLVXXLXXXXXXXXFXXXXPXXRVXRXSRGXVEEXCFRXXXXXXX
XXY

wherein X is any amino acid. A fragment of the epitope is one that a protein described herein specifically binds to.

More particularly, the protein may bind the following sequence of IGF-II, or a fragment thereof:

SETLCGGELVDTLQFVCGDRGFYFSRPASRVSRRSRGIVEECCFRSCDLA
LLETYCATPA

wherein the non-bolded residues may be substituted with conservative mutations.

The protein can bind to IGF-II and/or IGF-IIE, e.g., human IGF-II and/or IGF-IIE, with a binding affinity of at least 10⁵, 10⁶, 10⁷, 10⁸, 10⁹, 10¹⁰ and 10¹¹ M⁻¹. In one embodiment, the protein binds to human IGF-II and/or IGF-IIE with a K_{off} slower than 1×10⁻³, 5×10⁻⁴ s⁻¹, or 1×10⁻⁴ s⁻¹. In one embodiment, the protein binds to human IGF-II and/or IGF-IIE with a Kₒₙ faster than 1×10², 1×10³, or 5×10³ M⁻¹s⁻¹. In one embodiment, the protein inhibits human IGF-II and IGF-IIE activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. For example, the protein may modulate IGF-I receptor (IGF-1R) and/or an isoform of the insulin receptor (IR-A) activity, as well as IGF-II and IGF-IIE. The protein may inhibit IGF-1R, IR-A, and IGF-II and IGF-IIE activity. The affinity of the protein for human IGF-II and/or IGF-IIE can be characterized by a K_{D} of less than 100 nm, less than 10 nM, or less than 1 nM.

IGF-II/IGF-IIE binding proteins may be antibodies. IGF-II/IGF-IIE binding antibodies may have their HC and LC variable domain sequences included in a single polypeptide (e.g., scFv), or on different polypeptides (e.g., IgG or Fab).

In a preferred embodiment, the protein is an antibody (e.g., a human antibody) having the light and heavy chains of antibodies selected from the group consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the heavy chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having the light chain of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having light and heavy antibody variable regions of an antibody selected from the group consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a heavy chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having a light chain antibody variable region of an antibody selected from the group consisting of: M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs selected from the corresponding CDRs of the group of heavy chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In a preferred embodiment, the protein is an antibody (e.g., a human antibody) having one or more (e.g., 1, 2, or 3) heavy chain CDRs and one or more (e.g., 1, 2, or 3) light chain CDRs selected from the corresponding CDRs of the group of light chains consisting of M0033-E05, M0063-F02, M0064-E04, M0064-F02, M0068-E03, M0070-H08, M0072-006, M0072-E03, M0072-G06, germlined M0064-E04, germlined M0064-F02, DX-2647, and DX-2655.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab. In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In some embodiments, the affinity of the primate antibody for human IGF-II and IGF-IIE is characterized by a K_{D} of less than 1 nM.

In certain embodiments, the protein includes no sequences from mice or rabbits (e.g., is not a murine or rabbit antibody).

In certain embodiments, the protein may be capable of binding to tumor cells expressing IGF-II and/or IGF-IIE, e.g., to colorectal cell lines SW1116 (Grade A), SW480 (Grade B), HT29*, HT29, SW480, CaCO2, HCT116, SW620 (all Grade C), and COLO 205 (Grade D); breast cancer cell lines MCF-7* and 4T1; uterine cancer cell line SKUT-1 (mesodermal tumor), rhadbomyosarcoma cell lines, and hepatocellular carcinoma cell lines HepG2, HuH7 and Hep3B. In some embodiments, the protein is capable of binding lung fibroblasts or fibroblastic foci.

### IGF-II and IGF-IIE

Exemplary IGF-II and IGF-IIE sequences against which IGF-II/IGF-IIE binding proteins may be developed can include the human or mouse IGF-II and IGF-IIE amino acid sequences, a sequence that is 80%, 85%, 90%, 95%, 96%, 97%, 98%, or 99% identical to one of these sequences, or a fragment thereof, e.g., a fragment without the signal sequence or prodomain. The human and mouse IGF-II and IGF-IIE amino acid sequences, and the mRNA sequences encoding them, are illustrated below.

### IGF-II

>insulin-like growth factor II [human, small cell lung cancer cell line T3M-11, mRNA, 1322 nt] (truncated from ACCESSION S77035)

gctta ccgccccagt gagaccctgt gcggcgggga gctggtggac
accctccagt tcgtctgtgg ggaccgcggcttctacttca
gcaggcccgc aagccgtgtg agccgtcgca gccgtggcat
cgttgaggagtgctgtttcc gcagctgtga cctggccctc
ctggagacgt actgtgctac ccccgccaagtccgag

>insulin-like growth factor II; IGF-II [Homo sapiens]. (truncated from ACCESSION AAB34155)
ayrpsetleggelvdtlqfvcgdrgfyfsrpasrysrrsrgiveeccfrscdlalletycatpakse
>Mus musculus insulin-like growth factor 2, mRNA (cDNA clone MGC:60598 IMAGE:30013295), complete cds. (truncated from ACCESSION BC053489)

gc ttacggcccc ggagagactctgtgcggagg ggagcttgtt
gacacgcttc agtttgtctg ttcggaccgc
ggcttctacttcagcaggcc ttcaagccgt gccaaccgtc
gcagccgtgg catcgtggaa gagtgctgcttccgcagctg
cgacctggcc ctcctggaga catactgtgc cacccccgcc
aagtccgag

>Igf2 protein [Mus musculus]. (truncated from ACCESSION AAH53489)

aygpgetlcggelvdtlqfvcsdrgfyfsrpssranrrsrgiveeccf
rscdlalletycatpakse

IGF-IIE>insulin-like growth factor II [human, small cell lung cancer cell line T3M-11, mRNA, 1322 nt] (truncated from ACCESSION S77035)

atgggaa tcccaatggg gaagtcgatgctggtgcttc tcaccttctt
ggccttcgcc tcgtgctgca ttgctgctta
ccgccccagtgagaccctgt gcggcgggga gctggtggac
accctccagt tcgtctgtgg ggaccgcggcttctacttca
gcaggcccgc aagccgtgtg agccgtcgca gccgtggcat
cgttgaggagtgctgtttcc gcagctgtga cctggccctc
ctggagacgt actgtgctac ccccgccaagtccgagaggg
acgtgtcgac ccctccgacc gtgcttccgg acaacttccc
cagataccccgtgggcaagt tcttccaata tgacacctgg
aagcagtcca cccagcgcct gcgcaggggcctgcctgccc
tcctgcgtgc ccgccggggt cacgtgctcg ccaaggagct
cgaggcgttcagggaggcca aacgtcaccg tcccctgatt
gctctaccca cccaagaccc cgcccacgggggcgcccccc
cagagatggc cagcaatcgg aag

>insulin-like growth factor II; IGF-II [Homo sapiens]. (ACCESSION AAB34155)

mgipmgksml vlltflafas cciaayrpse tlcggelvdt
lqfvcgdrgf yfsrpasrvsrrsrgiveec cfrscdlall
etycatpaks erdvstpptv lpdnfprypv
gkffqydtwkqstqrlrrgl pallrarrgh vlakeleafr
eakrhrplia lptqdpahgg appemasnrk

>Mus musculus insulin-like growth factor 2, mRNA (cDNA clone MGC:60598 IMAGE: 30013295), complete cds. (truncated from ACCESSION BC053489)

atgg ggatcccagt ggggaagtcg atgttggtgcttctcatctc
tttggccttc gccttgtgct gcatcgctgc ttacggcccc
ggagagactctgtgcggagg ggagcttgtt gacacgcttc
agtttgtctg ttcggaccgc ggcttctacttcagcaggcc
ttcaagccgt gccaaccgtc gcagccgtgg catcgtggaa
gagtgctgcttccgcagctg cgacctggcc ctcctggaga
catactgtgc cacccccgcc aagtccgagagggacgtgtc
tacctctcag gccgtacttc cggacgactt ccccagatac
cccgtgggcaagttcttcca atatgacacc tggagacagt
ccgcgggacg cctgcgcaga ggcctgcctgccctcctgcg
tgcccgccgg ggtcgcatgc ttgccaaaga gctcaaagag
ttcagagaggccaaacgtca tcgtcccctg atcgtgttac
cacccaaaga ccccgcccac gggggagcctcttcggagat
gtccagcaac catcag

>Igf2 protein [Mus musculus]. (ACCESSION AAH53489)

mgipvgksml vllislafal cciaaygpge tlcggelvdt
lqfvcsdrgf yfsrpssranrrsrgiveec cfrscdlall
etycatpaks erdvstsqav lpddfprypv
gkffqydtwrqsagrlrrgl pallrarrgr mlakelkefr
eakrhrpliv lppkdpahgg assemssnhq

### Display Libraries

A display library is a collection of entities; each entity includes an accessible polypeptide component and a recoverable component that encodes or identifies the polypeptide component. The polypeptide component is varied so that different amino acid sequences are represented. The polypeptide component can be of any length, e.g. from three amino acids to over 300 amino acids. A display library entity can include more than one polypeptide component, for example, the two polypeptide chains of an sFab. In one exemplary implementation, a display library can be used to identify proteins that bind to both IGF-II and IGF-IIE. In a selection, the polypeptide component of each member of the library is probed with IGF-II and/or IGF-IIE (or fragment thereof) and if the polypeptide component binds to the IGF-II and/or IGF-IIE, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

Phage Display: The protein component is typically covalently linked to a bacteriophage coat protein. The linkage results from translation of a nucleic acid encoding the protein component fused to the coat protein. The linkage can include a flexible peptide linker, a protease site, or an amino acid incorporated as a result of suppression of a stop codon. Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol Today 2:371-8 and Hoet et al. (2005) Nat. Biotechnol. 23(3)344-8. Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced.

Other Display Formats. Other display formats include cell based display (see, e.g., WO 03/029456), protein-nucleic acid fusions (see, e.g., U.S. Pat. No. 6,207,446), ribosome display (See, e.g., Mattheakis et al. (1994) Proc. Natl. Acad. Sci. USA 91:9022 and Hanes et al. (2000) Nat. Biotechnol. 18:1287-92; Hanes et al. (2000) Methods Enzymol. 328:404-30; and Schaffitzel et al. (1999) J Immunol Methods. 231(1-2):119-35), and E. coli periplasmic display (J Immunol Methods. 2005 Nov. 22;PMID: 16337958).

Scaffolds. Scaffolds useful for display include: antibodies (e.g., Fab fragments, single chain FIT molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin and heat shock proteins; intracellular signaling domains (such as SH2 and SH3 domains); linear and constrained peptides; and linear peptide substrates. Display libraries can include synthetic and/or natural diversity. See, e.g., US 2004-0005709.

Display technology can also be used to obtain binding proteins (e.g., antibodies) that bind particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

Iterative Selection. In one preferred embodiment, display library technology is used in an iterative mode. A first display library is used to identify one or more binding proteins for a target. These identified binding proteins are then varied using a mutagenesis method to form a second display library. Higher affinity binding proteins are then selected from the second library, e.g., by using higher stringency or more competitive binding and washing conditions.

In some implementations, the mutagenesis is targeted to regions at the binding interface. If, for example, the identified binding proteins are antibodies, then mutagenesis can be directed to the CDR regions of the heavy or light chains as described herein. Further, mutagenesis can be directed to framework regions near or adjacent to the CDRs. In the case of antibodies, mutagenesis can also be limited to one or a few of the CDRs, e.g., to make precise step-wise improvements. Exemplary mutagenesis techniques include: error-prone PCR, recombination, DNA shuffling, site-directed mutagenesis and cassette mutagenesis.

In one example of iterative selection, the methods described herein are used to first identify a protein from a display library that binds both IGF-II and IGF-IIE, with at least a minimal binding specificity for a target or a minimal activity, e.g., an equilibrium dissociation constant for binding of less than 1 nM, 10 nM, or 100 nM. The nucleic acid sequence encoding the initial identified proteins are used as a template nucleic acid for the introduction of variations, e.g., to identify a second protein that has enhanced properties (e.g., binding affinity, kinetics, or stability) relative to the initial protein.

Off-Rate Selection. Since a slow dissociation rate can be predictive of high affinity, particularly with respect to interactions between polypeptides and their targets, the methods described herein can be used to isolate binding proteins with a desired (e.g., reduced) kinetic dissociation rate for a binding interaction to a target.

To select for slow dissociating binding proteins from a display library, the library to is contacted to an immobilized target. The immobilized target is then washed with a first solution that removes non-specifically or weakly bound biomolecules. Then the bound binding proteins are eluted with a second solution that includes a saturating amount of free target or a target specific high-affinity competing monoclonal antibody, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

Selecting or Screening for Specificity. The display library screening methods described herein can include a selection or screening process that discards display library members that bind to a non-target molecule. Examples of non-target molecules include streptavidin on magnetic beads, blocking agents such as bovine serum albumin, non-fat bovine milk, soy protein, any capturing or target immobilizing monoclonal antibody, or non-transfected cells which do not express the target.

In one implementation, a so-called “negative selection” step is used to discriminate between the target and related non-target molecule and a related, but distinct non-target molecules. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target as well as for cross species reactivity to related targets or subunits of the target (e.g., IGF-II and/or IGF-IIE) and also under different condition such as pH6 or pH 7.5. The non-target and target binding data are compared (e.g., using a computer and software) to identify library members that specifically bind to the target.

### Other Exemplary Expression Libraries

Other types of collections of proteins (e.g., expression libraries) can be used to identify proteins with a particular property (e.g., ability to bind IGF-II and IGF-IIE), including, e.g., protein arrays of antibodies (see, e.g., De Wildt et al. (2000) Nat. Biotechnol. 18:989-994), lambda gt11 libraries, two-hybrid libraries and so forth.

### Exemplary Libraries

It is possible to immunize a non-human primate and recover primate antibody genes that can be displayed on phage (see below). From such a library, one can select antibodies that bind the antigen used in immunization. See, for example, Vaccine. (2003) 22(2):257-67 or Immunogenetics. (2005) 57(10):730-8. Thus one could obtain primate antibodies that bind and inhibit IGF-II and IGF-IIE by immunizing a chimpanzee or macaque and using a variety of means to select or screen for primate antibodies that bind and inhibit IGF-II and IGF-IIE. One can also make chimeras of primatized Fabs with human constant regions, see Curr Opin Mol. Ther. (2004) 6(6):675-83. “PRIMATIZED antibodies, genetically engineered from cynomolgus macaque monkey and human components, are structurally indistinguishable from human antibodies. They may, therefore, be less likely to cause adverse reactions in humans, making them potentially suited for long-term, chronic treatment” Curr Opin Investig Drugs. (2001) 2(5):635-8.

One exemplary type of library presents a diverse pool of polypeptides, each of which includes an immunoglobulin domain, e.g., an immunoglobulin variable domain. Of interest are display libraries where the members of the library include primate or “primatized” (e.g., such as human, non-human primate or “humanized”) immunoglobin domains (e.g., immunoglobin variable domains) or chimeric primatized Fabs with human constant regions. Human or humanized immunoglobin domain libraries may be used to identify human or “humanized” antibodies that, for example, recognize human antigens. Because the constant and framework regions of the antibody are human, these antibodies may avoid themselves being recognized and targeted as antigens when administered to humans. The constant regions may also be optimized to recruit effector functions of the human immune system. The in vitro display selection process surmounts the inability of a normal human immune system to generate antibodies against self-antigens.

A typical antibody display library displays a polypeptide that includes a VH domain and a VL domain. An “immunoglobulin domain” refers to a domain from the variable or constant domain of immunoglobulin molecules Immunoglobulin domains typically contain two β-sheets formed of about seven β-strands, and a conserved disulphide bond (see, e.g., A. F. Williams and A. N. Barclay, 1988, Ann. Rev. Immunol. 6:381-405). The display library can display the antibody as a Fab fragment (e.g., using two polypeptide chains) or a single chain FIT (e.g., using a single polypeptide chain). Other formats can also be used.

As in the case of the Fab and other formats, the displayed antibody can include one or more constant regions as part of a light and/or heavy chain. In one embodiment, each chain includes one constant region, e.g., as in the case of a Fab. In other embodiments, additional constant regions are displayed.

Antibody libraries can be constructed by a number of processes (see, e.g., de Haard et al., 1999, J. Biol. Chem. 274:18218-30; Hoogenboom et al., 1998, Immunotechnology 4:1-20; Hoogenboom et al., 2000, Immunol. Today 21:371-378, and Hoet et al. (2005) Nat. Biotechnol. 23(3)344-8. Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. The variation(s) may be introduced into all three CDRs of a given variable domain, or into CDR1 and CDR2, e.g., of a heavy chain variable domain. Any combination is feasible. In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al., 2000, J. Mol. Biol. 296:57-86 describe a method for constructing CDR encoding oligonucleotides using trinucleotide synthesis and a template with engineered restriction sites for accepting the oligonucleotides.

In another process, an animal, e.g., a rodent, is immunized with IGF-II and IGF-IIE. The animal is optionally boosted with the antigen to further stimulate the response. Then spleen cells are isolated from the animal, and nucleic acid encoding VH and/or VL domains is amplified and cloned for expression in the display library.

In yet another process, antibody libraries are constructed from nucleic acid amplified from naïve germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

Nucleic acid encoding immunoglobulin domains can be obtained from the immune cells of, e.g., a primate (e.g., a human), mouse, rabbit, camel, or rodent. In one example, the cells are selected for a particular property. B cells at various stages of maturity can be selected. In another example, the B cells are naïve.

In one embodiment, fluorescent-activated cell sorting (FACS) is used to sort B cells that express surface-bound IgM, IgD, or IgG molecules. Further, B cells expressing different isotypes of IgG can be isolated. In another preferred embodiment, the B or T cells are cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin, or pokeweed mitogen.

In another embodiment, the cells are isolated from a subject that has a disease of condition described herein, e.g., systemic sclerosis-associate pulmonary fibrosis.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al., 2001, J. Immunol. 166:2228). In another embodiment, the cells are naïve.

The nucleic acid encoding an immunoglobulin variable domain can be isolated from a natural repertoire by the following exemplary method. First, RNA is isolated from the immune cell. Full length (i.e., capped) mRNAs are separated (e.g. by degrading uncapped RNAs with calf intestinal phosphatase). The cap is then removed with tobacco acid pyrophosphatase and reverse transcription is used to produce the cDNAs.

The reverse transcription of the first (antisense) strand can be done in any manner with any suitable primer. See, e.g., de Haard et al., 1999, J. Biol. Chem. 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. In another embodiment, poly-dT primers may be used (and may be preferred for the heavy-chain genes).

A synthetic sequence can be ligated to the 3′ end of the reverse transcribed strand. The synthetic sequence can be used as a primer binding site for binding of the forward primer during PCR amplification after reverse transcription. The use of the synthetic sequence can obviate the need to use a pool of different forward primers to fully capture the available diversity.

The variable domain-encoding gene is then amplified, e.g., using one or more rounds. If multiple rounds are used, nested primers can be used for increased fidelity. The amplified nucleic acid is then cloned into a display library vector.

### Secondary Screening Methods

After selecting candidate library members that bind to a target, each candidate library member can be further analyzed, e.g., to further characterize its binding properties for the target, e.g., IGF-II and/or IGF-IIE, or for binding to another protein, e.g., another IGF protein, such as IGF-I. Each candidate library member can be subjected to one or more secondary screening assays. The assay can be for a binding property, a catalytic property, an inhibitory property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use a display library member directly, a recombinant polypeptide produced from the nucleic acid encoding the selected polypeptide, or a synthetic peptide synthesized based on the sequence of the selected polypeptide. In the case of selected Fabs, the Fabs can be evaluated or can be modified and produced as intact IgG proteins. Exemplary assays for binding properties include the following.

ELISA. Binding proteins can be evaluated using an ELISA assay. For example, each protein is contacted to a microtitre plate whose bottom surface has been coated with the target, e.g., a limiting amount of the target. The plate is washed with buffer to remove non-specifically bound polypeptides. Then the amount of the binding protein bound to the target on the plate is determined by probing the plate with an antibody that can recognize the binding protein, e.g., a tag or constant portion of the binding protein. The antibody is linked to a detection system (e.g., an enzyme such as alkaline phosphatase or horse radish peroxidase (HRP) which produces a colorimetric product when appropriate substrates are provided).

Homogeneous Binding Assays. The ability of a binding protein described herein to bind a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means, e.g., using a fluorimeter. By titrating the amount of the first or second binding molecule, a binding curve can be generated to estimate the equilibrium binding constant.

Another example of a homogenous assay is ALPHASCREEN™ (Packard Bioscience, Meriden Conn.). ALPHASCREEN™ uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

Surface Plasmon Resonance (SPR). The interaction of binding protein and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether, 1988, Surface Plasmons Springer Verlag; Sjolander and Urbaniczky, 1991, Anal. Chem. 63:2338-2345; Szabo et al., 1995, Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{D}), and kinetic parameters, including Kₒₙ and K_{off}, for the binding of a binding protein to a target. Such data can be used to compare different biomolecules. For example, selected proteins from an expression library can be compared to identify proteins that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

Cellular Assays. Binding proteins can be screened for ability to bind to cells which transiently or stably express and display the target of interest on the cell surface. For example, IGF-II/IGF-IIE binding proteins can be fluorescently labeled and binding to IGF-II and/or IGF-IIE in the presence of absence of antagonistic antibody can be detected by a change in fluorescence intensity using flow cytometry e.g., a FACS machine.

### Other Exemplary Methods for Obtaining IGF-II/IGF-IIE Binding Proteins

In addition to the use of display libraries, other methods can be used to obtain an IGF-II/IGF-IIE binding protein (e.g., antibody). For example, IGF-II and/or IGF-IIE protein or a region thereof can be used as an antigen in a non-human animal, e.g., a rodent.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies (Mabs) derived from the genes with the desired specificity may be produced and selected. See, e.g., XENOMOUSE™, Green et al., 1994, Nat. Gen. 7:13-21; U.S. 2003-0070185, WO 96/34096, published Oct. 31, 1996, and PCT Application No. PCT/US96/05928, filed Apr. 29, 1996.

In another embodiment, a monoclonal antibody is obtained from the non-human animal, and then modified, e.g., humanized or deimmunized. Winter describes a CDR-grafting method that may be used to prepare the humanized antibodies (UK Patent Application GB 2188638A, filed on Mar. 26, 1987; U.S. Pat. No. 5,225,539. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen.

Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. Nos. 5,585,089, U.S. Pat. No. 5,693,761 and U.S. Pat. No. 5,693,762. Those methods include isolating, manipulating, and expressing the nucleic acid sequences that encode all or part of immunoglobulin Fv variable regions from at least one of a heavy or light chain. Numerous sources of such nucleic acid are available. For example, nucleic acids may be obtained from a hybridoma producing an antibody against a predetermined target, as described above. The recombinant DNA encoding the humanized antibody, or fragment thereof, can then be cloned into an appropriate expression vector.

Reducing Immunogenicity of IGF-II/IGF-IIE Binding Proteins

Immunoglobin IGF-II/IGF-IIE binding proteins (e.g., IgG or Fab IGF-II/IGF-IIE binding proteins) may be modified to reduce immunogenicity. Reduced immunogenicity is desirable in IGF-II/IGF-IIE binding proteins intended for use as therapeutics, as it reduces the chance that the subject will develop an immune response against the therapeutic molecule. Techniques useful for reducing immunogenicity of IGF-II/IGF-IIE binding proteins include deletion/modification of potential human T cell epitopes and ‘germlining’ of sequences outside of the CDRs (e.g., framework and Fc).

An IGF-II/IGF-IIE-binding antibody may be modified by specific deletion of human T cell epitopes or “deimmunization” by the methods disclosed in WO 98/52976 and WO 00/34317. Briefly, the heavy and light chain variable regions of an antibody are analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible conservative substitutions are made, often but not exclusively, an amino acid common at this position in human germline antibody sequences may be used. Human germline sequences are disclosed in Tomlinson, I. A. et al., 1992, J. Mol. Biol. 227:776-798; Cook, G. P. et al., 1995, Immunol. Today Vol. 16 (5): 237-242; Chothia, D. et al., 1992, J. Mol. Bio. 227:799-817. The V BASE directory provides a comprehensive directory of human immunoglobulin variable region sequences (compiled by Tomlinson, I. A. et al. MRC Centre for Protein Engineering, Cambridge, UK). After the deimmunizing changes are identified, nucleic acids encoding V_{H} and V_{L} can be constructed by mutagenesis or other synthetic methods (e.g., de novo synthesis, cassette replacement, and so forth). Mutagenized variable sequence can, optionally, be fused to a human constant region, e.g., human IgG1 or κ constant regions.

In some cases a potential T cell epitope will include residues which are known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs were eliminated by substitutions outside the CDRs. In some cases, an alteration within a CDR is the only option, and thus variants with and without this substitution should be tested. In other cases, the substitution required to remove a potential T cell epitope is at a residue position within the framework that might be critical for antibody binding. In these cases, variants with and without this substitution should be tested. Thus, in some cases several variant deimmunized heavy and light chain variable regions were designed and various heavy/light chain combinations tested in order to identify the optimal deimmunized antibody. The choice of the final deimmunized antibody can then be made by considering the binding affinity of the different variants in conjunction with the extent of deimmunization, i.e., the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

IGF-II/IGF-IIE binding antibodies are “germlined” by reverting one or more non-germline amino acids in framework regions to corresponding germline amino acids of the antibody, so long as binding properties are substantially retained. Similar methods can also be used in the constant region, e.g., in constant immunoglobulin domains.

Antibodies that bind to both IGF-II and IGF-IIE e.g., an antibody described herein, may be modified in order to make the variable regions of the antibody more similar to one or more germline sequences. For example, an antibody can include one, two, three, or more amino acid substitutions, e.g., in a framework, CDR, or constant region, to make it more similar to a reference germline sequence. One exemplary germlining method can include identifying one or more germline sequences that are similar (e.g., most similar in a particular database) to the sequence of the isolated antibody. Mutations (at the amino acid level) are then made in the isolated antibody, either incrementally or in combination with other mutations. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a framework and/or constant region. For example, a germline framework and/or constant region residue can be from a germline sequence that is similar (e.g., most similar) to the non-variable region being modified. After mutagenesis, activity (e.g., binding or other functional activity) of the antibody can be evaluated to determine if the germline residue or residues are tolerated (i.e., do not abrogate activity). Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may including using two germline sequences that separately contribute to the amino-terminal portion and the carboxy-terminal portion. In other implementations more than one or two germline sequences are used, e.g., to form a consensus sequence.

In one embodiment, with respect to a particular reference variable domain sequence, e.g., a sequence described herein, a related variable domain sequence has at least 30, 40, 50, 60, 70, 80, 90, 95 or 100% of the CDR amino acid positions that are not identical to residues in the reference CDR sequences, residues that are identical to residues at corresponding positions in a human germline sequence (i.e., an amino acid sequence encoded by a human germline nucleic acid).

In one embodiment, with respect to a particular reference variable domain sequence, e.g., a sequence described herein, a related variable domain sequence has at least 30, 50, 60, 70, 80, 90 or 100% of the FR regions identical to FR sequence from a human germline sequence, e.g., a germline sequence related to the reference variable domain sequence.

Accordingly, it is possible to isolate an antibody which has similar activity to a given antibody of interest, but is more similar to one or more germline sequences, particularly one or more human germline sequences. For example, an antibody can be at least 90, 91, 92, 93, 94, 95, 96, 97, 98, 99, or 99.5% identical to a germline sequence in a region outside the CDRs (e.g., framework regions). Further, an antibody can include at least 1, 2, 3, 4, or 5 germline residues in a CDR region, the germline residue being from a germline sequence of similar (e.g., most similar) to the variable region being modified. Germline sequences of primary interest are human germline sequences. The activity of the antibody (e.g., the binding activity as measured by K_{A}) can be within a factor or 100, 10, 5, 2, 0.5, 0.1, and 0.001 of the original antibody.

Germline sequences of human immunoglobin genes have been determined and are available from a number of sources, including the international ImMunoGeneTics Information System® (IMGT), available via the world wide web at imgt.cines.fr, and the V BASE directory (compiled by Tomlinson, I. A. et al. MRC Centre for Protein Engineering, Cambridge, UK, available via the world wide web at vbase.mrc-cpe.cam.ac.uk).

Exemplary germline reference sequences for Vₖₐₚₚₐ include: O12/O2, O18/O8, A20, A30, L14, L1, L15, L4/18a, L5/L19, L8, L23, L9, L24, L11, L12, O11/O1, A17, A1, A18, A2, A19/A3, A23, A27, A11, L2/L16, L6, L20, L25, B3, B2, A26/A10, and A14. See, e.g., Tomlinson et al., 1995, EMBO J. 14(18):4628-3.

A germline reference sequence for the HC variable domain can be based on a sequence that has particular canonical structures, e.g., 1-3 structures in the H1 and H2 hypervariable loops. The canonical structures of hypervariable loops of an immunoglobulin variable domain can be inferred from its sequence, as described in Chothia et al., 1992, J. Mol. Biol. 227:799-817; Tomlinson et al., 1992, J. Mol. Biol. 227:776-798); and Tomlinson et al., 1995, EMBO J. 14(18):4628-38. Exemplary sequences with a 1-3 structure include: DP-1, DP-8, DP-12, DP-2, DP-25, DP-15, DP-7, DP-4, DP-31, DP-32, DP-33, DP-35, DP-40, 7-2, hv3005, hv3005f3, DP-46, DP-47, DP-58, DP-49, DP-50, DP-51, DP-53, and DP-54.

### Protein Production

Standard recombinant nucleic acid methods can be used to express a protein that binds to both IGF-II and IGF-IIE. Generally, a nucleic acid sequence encoding the protein is cloned into a nucleic acid expression vector. Of course, if the protein includes multiple polypeptide chains, each chain can be cloned into an expression vector, e.g., the same or different vectors, that are expressed in the same or different cells.

Antibody Production. Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

Antibodies can also be produced in eukaryotic cells. In one embodiment, the antibodies (e.g., scFv's) are expressed in a yeast cell such as Pichia (see, e.g., Powers et al., 2001, J. Immunol. Methods. 251:123-35), Hanseula, or Saccharomyces.

In one preferred embodiment, antibodies are produced in mammalian cells. Preferred mammalian host cells for expressing the clone antibodies or antigen-binding fragments thereof include Chinese Hamster Ovary (CHO cells) (including dhfr− CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601 621), lymphocytic cell lines, e.g., NS0 myeloma cells and SP2 cells, COS cells, HEK293T cells (J. Immunol. Methods (2004) 289(1-2):65-80.), and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G coupled matrix.

For antibodies that include an Fc domain, the antibody production system may produce antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcg receptors and complement C1q (Burton and Woof, 1992, Adv. Immunol. 51:1-84; Jefferis et al., 1998, Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

### Characterization of IGF-II/IGF-IIE Binding Proteins

EC₅₀ (Effective Concentration 50%) value for that binding protein. Within a series or group of binding proteins, those having lower IC₅₀ or EC₅₀ values are considered more potent inhibitors of IGF-II or IGF-IIE than those binding proteins having higher IC₅₀ or EC₅₀ values. Exemplary binding proteins have an IC₅₀ value of less than 800 nM, 400 nM, 100 nM, 25 nM, 5 nM, or 1 nM, e.g., as measured in an in vitro assay for inhibition of IGF-II or IGF-IIE activity when the IGF-II or IGF-IIE is at 2 pM.

IGF-II/IGF-IIE binding proteins may also be characterized with reference to the activity of IGF-II/IGF-IIE on IGF receptor type I (IGF-1R or IGF-IR) and IR-A (the A isoform of the insulin receptor) signaling events.

The binding proteins can also be evaluated for selectivity toward IGF-II and/or IGF-IIE. For example, an IGF-II/IGF-IIE binding protein can be assayed for its potency toward IGF-II and/or IGF-IIE and a panel of IGF-II's and an IC₅₀ value or EC₅₀ value can be determined for each IGF. In one embodiment, a compound that demonstrates a low IC₅₀ value or EC₅₀ value for the IGF-II or IGF-IIE, and a higher IC₅₀ value or EC₅₀ value, e.g., at least 2-, 5-, or 10-fold higher, for another IGF within the test panel is considered to be selective toward IGF-II and/or IGF-IIE.

IGF-II/IGF-IIE binding proteins can be evaluated for their ability to inhibit IGF-II and/or IGF-IIE in a cell based assay. The expansion of tumor cells inside a three-dimensional collagen-matrix can be significantly enhanced in response to IGF-II and/or IGF-IIE overexpression (Hotary et al., 2003 Cell 114:33-45). Addition of an IGF-II/IGF-IIE binding protein to this assay can be used to determine the inhibitory properties and/or other characteristics of the protein.

A pharmacokinetics study in rat, mice, or monkey can be performed with IGF-II/IGF-IIE binding proteins for determining IGF-II and/or IGF-IIE half-life in the serum. Likewise, the effect of the binding protein can be assessed in vivo, e.g., in an animal model for a disease, for use as a therapeutic, for example, to treat a disease or condition described herein, e.g., systemic sclerosis-associated pulmonary fibrosis.

### Pharmaceutical Compositions

Proteins (e.g., binding proteins) that bind to both or either IGF-II and/or IGF-IIE (e.g., human IGF-II and/or IGF-IIE) and, e.g., include at least one immunoglobin variable region can be used in methods for treating (or preventing) SSc-associated pulmonary fibrosis. The binding proteins can be present in a composition, e.g., a pharmaceutically acceptable composition or pharmaceutical composition, which includes an IGF-II/IGF-IIE-binding protein, e.g., an antibody molecule, other polypeptide or peptide identified as binding to IGF-II and IGF-IIE, as described herein. The IGF-II/IGF-IIE binding protein can be formulated together with a pharmaceutically acceptable carrier. Pharmaceutical compositions include therapeutic compositions and diagnostic compositions, e.g., compositions that include labeled IGF-II/IGF-IIE binding proteins for in vivo imaging, and compositions that include labeled IGF-II/IGF-IIE binding proteins for treating (or preventing) SSc-associated pulmonary fibrosis.

A pharmaceutically acceptable carrier includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal, or epidermal administration (e.g., by injection or infusion), although carriers suitable for inhalation and intranasal administration are also contemplated. Depending on the route of administration, the IGF-II/IGF-IIE binding protein may be coated in a material to protect the compound from the action of acids and other natural conditions that may inactivate the compound.

A pharmaceutically acceptable salt is a salt that retains the desired biological activity of the parent compound and does not impart any undesired toxicological effects (see e.g., Berge, S. M., et al., 1977, J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like.

The compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Many compositions are in the form of injectable or infusible solutions, such as compositions similar to those used for administration of humans with antibodies. An exemplary mode of administration is parenteral (e.g., intravenous, subcutaneous, intraperitoneal, intramuscular). In one embodiment, the IGF-II/IGF-IIE binding protein is administered by intravenous infusion or injection. In another preferred embodiment, the IGF-II/IGF-IIE binding protein is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable to high drug concentration. Sterile injectable solutions can be prepared by incorporating the binding protein in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An IGF-II/IGF-IIE binding protein can be administered by a variety of methods, although for many applications, the preferred route/mode of administration is intravenous injection or infusion. For example, for therapeutic applications, the IGF-II/IGF-IIE binding protein can be administered by intravenous infusion at a rate of less than 30, 20, 10, 5, or 1 mg/min to reach a dose of about 1 to 100 m g/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are available. See, e.g., Sustained and Controlled Release Drug Delivery Systems, J.R. Robinson, ed., 1978, Marcel Dekker, Inc., New York.

Pharmaceutical compositions can be administered with medical devices. For example, in one embodiment, a pharmaceutical composition disclosed herein can be administered with a device, e.g., a needleless hypodermic injection device, a pump, or implant.

In certain embodiments, an IGF-II/IGF-IIE binding protein can be formulated to ensure proper distribution in vivo. For example, the blood-brain barrier (BBB) excludes many highly hydrophilic compounds. To ensure that the therapeutic compounds disclosed herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V.V. Ranade, 1989, J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a binding protein (e.g., an antibody) disclosed herein is 0.1-20 mg/kg, more preferably 1-10 mg/kg. An anti-IGF-II/IGF-IIE antibody can be administered, e.g., by intravenous infusion, e.g., at a rate of less than 30, 20, 10, 5, or 1 mg/min to reach a dose of about 1 to 100 mg/m² or about 5 to 30 mg/m². For binding proteins smaller in molecular weight than an antibody, appropriate amounts can be proportionally less. Dosage values may vary with the type and severity of the condition to be alleviated. For a particular subject, specific dosage regimens can be adjusted over time according to the individual need and the professional judgment of the person administering or supervising the administration of the compositions.

The pharmaceutical compositions disclosed herein may include a “therapeutically effective amount” or a “prophylactically effective amount” of an IGF-II/IGF-IIE binding protein disclosed herein. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition is outweighed by the therapeutically beneficial effects.

A “therapeutically effective dosage” preferably modulates a measurable parameter, e.g., levels of circulating IgG antibodies by a statistically significant degree or at least about 20%, more preferably by at least about 40%, even more preferably by at least about 60%, and still more preferably by at least about 80% relative to untreated subjects. The ability of a compound to modulate a measurable parameter, e.g., a disease-associated parameter, can be evaluated in an animal model system predictive of efficacy in human disorders and conditions, e.g., systemic sclerosis-associated pulmonary fibrosis. Alternatively, this property of a composition can be evaluated by examining the ability of the compound to modulate a parameter in vitro.

A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

### Stabilization and Retention

In one embodiment, an IGF-II/IGF-IIE binding protein is physically associated with a moiety that improves its stabilization and/or retention in circulation, e.g., in blood, serum, lymph, or other tissues, e.g., by at least 1.5, 2, 5, 10, or 50 fold. For example, an IGF-II/IGF-IIE binding protein can be associated with a polymer, e.g., a substantially non-antigenic polymer, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used. For example, an IGF-II/IGF-IIE binding protein can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

An IGF-II/IGF-IIE binding protein can also be associated with a carrier protein, e.g., a serum albumin, such as a human serum albumin. For example, a translational fusion can be used to associate the carrier protein with the IGF-II/IGF-IIE binding protein.

### Kits

An IGF-II/IGF-IIE binding protein described herein can be provided in a kit, e.g., as a component of a kit. For example, the kit includes (a) an IGF-II/IGF-IIE binding protein, e.g., a composition (e.g., a pharmaceutical composition) that includes an IGF-II/IGF-IIE binding protein, and, optionally (b) informational material. The informational material can be descriptive, instructional, marketing or other material that relates to the methods described herein and/or the use of an IGF-II/IGF-IIE binding protein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to using the binding protein to treat, prevent, or diagnosis of disorders and conditions, e.g., systemic sclerosis-associated pulmonary fibrosis.

In one embodiment, the informational material can include instructions to administer an IGF-II/IGF-IIE binding protein in a suitable manner to perform the methods described herein, e.g., in a suitable dose, dosage form, or mode of administration (e.g., a dose, dosage form, or mode of administration described herein). In another embodiment, the informational material can include instructions to administer an IGF-II/IGF-IIE binding protein to a suitable subject, e.g., a human, e.g., a human having, or at risk for, a disorder or condition described herein, e.g., systemic sclerosis-associated pulmonary fibrosis. For example, the material can include instructions to administer an IGF-II/IGF-IIE binding protein to a patient with a disorder or condition described herein, e.g., systemic sclerosis-associated pulmonary fibrosis. The informational material of the kits is not limited in its form. In many cases, the informational material, e.g., instructions, is provided in print but may also be in other formats, such as computer readable material.

An IGF-II/IGF-IIE binding protein can be provided in any form, e.g., liquid, dried or lyophilized form. It is preferred that an IGF-II/IGF-IIE binding protein be substantially pure and/or sterile. When an IGF-II/IGF-IIE binding protein is provided in a liquid solution, the liquid solution preferably is an aqueous solution, with a sterile aqueous solution being preferred. When an IGF-II/IGF-IIE binding protein is provided as a dried form, reconstitution generally is by the addition of a suitable solvent. The solvent, e.g., sterile water or buffer, can optionally be provided in the kit.

The kit can include one or more containers for the composition containing an IGF-II/IGF-IIE binding protein. In some embodiments, the kit contains separate containers, dividers or compartments for the composition and informational material. For example, the composition can be contained in a bottle, vial, or syringe, and the informational material can be contained association with the container. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an IGF-II/IGF-IIE binding protein. For example, the kit includes a plurality of syringes, ampules, foil packets, or blister packs, each containing a single unit dose of an IGF-II/IGF-IIE binding protein. The containers of the kits can be air tight, waterproof (e.g., impermeable to changes in moisture or evaporation), and/or light-tight.

The kit optionally includes a device suitable for administration of the composition, e.g., a syringe, inhalant, dropper (e.g., eye dropper), swab (e.g., a cotton swab or wooden swab), or any such delivery device. In one embodiment, the device is an implantable device that dispenses metered doses of the binding protein. The disclosure also features a method of providing a kit, e.g., by combining components described herein.

### Treatments

Proteins that bind to both IGF-II/IGF-IIE, and identified by the method described herein and/or detailed herein, have therapeutic and prophylactic utilities, particularly in human subjects. These binding proteins are administered to a subject to treat, prevent, and/or diagnose a variety of disorders, including e.g., systemic sclerosis-associated pulmonary fibrosis, or even to cells in culture, e.g., in vitro or ex vivo. Treating includes administering an amount effective to alleviate, relieve, alter, remedy, ameliorate, improve or affect the disorder, the symptoms of the disorder or the predisposition toward the disorder. The treatment may also delay onset, e.g., prevent onset, or prevent deterioration of a disease or condition.

As used herein, an amount of a target-binding agent effective to prevent a disorder, or a prophylactically effective amount of the binding agent refers to an amount of a target binding agent, e.g., an IGF-II/IGF-IIE binding protein, e.g., an anti-IGF-II/IGF-IIE antibody described herein, which is effective, upon single- or multiple-dose administration to the subject, for preventing or delaying the occurrence of the onset or recurrence of a disorder, e.g., a disorder described herein, e.g., systemic sclerosis-associated pulmonary fibrosis.

Methods of administering IGF-II/IGF-IIE binding proteins and other agents are also described in “Pharmaceutical Compositions.” Suitable dosages of the molecules used can depend on the age and weight of the subject and the particular drug used. The binding proteins can be used as competitive agents to inhibit, reduce an undesirable interaction, e.g., between a natural or pathological agent and the IGF-II/IGF-IIE. The dose of the IGF-II/IGF-IIE binding protein can be the amount sufficient to block 90%, 95%, 99%, or 99.9% of the activity of IGF-II/IGF-IIE in the patient, especially at the site of disease. Depending on the disease, this may require 0.1, 1.0, 3.0, 6.0, or 10.0 mg/Kg. For an IgG having a molecular mass of 150,000 g/mole (two binding sites), these doses correspond to approximately 18 nM, 180 nM, 540 nM, 1.08 μM, and 1.8 μM of binding sites for a 5 L blood volume.

In one embodiment, the IGF-II/IGF-IIE binding proteins are used to inhibit an activity (e.g., inhibit at least one activity of or reduce collagen and/or fibronectin production) of a cell, e.g., a lung fibroblast in vivo. The binding proteins can be used by themselves or conjugated to an agent, e.g., a cytotoxic drug, cytotoxin enzyme, or radioisotope. This method includes: administering the binding protein alone or attached to an agent (e.g., a cytotoxic drug), to a subject requiring such treatment. For example, IGF-II/IGF-IIE binding proteins that do not substantially inhibit IGF-II/IGF-IIE may be used to deliver nanoparticles containing agents, such as toxins, to IGF-II/IGF-IIE associated cells or tissues, e.g., lung fibroblasts or fibroblastic foci from SSc lungs.

Because the IGF-II/IGF-IIE binding proteins recognize IGF-II/IGF-IIE-expressing cells and can bind to cells that are associated with (e.g., in proximity of or intermingled with) a lung, e.g., lung fibroblasts, e.g., fibroblasts from SSc lungs, or fibroblastic foci, IGF-II/IGF-IIE binding proteins can be used to inhibit (e.g., inhibit at least one activity or reduce collagen and/or fibronectin production) any such cells and inhibit or decrease fibrosis. Reducing IGF-II/IGF-IIE activity of or near lung fibrobalsts or fibroblastic foci of lungs, e.g., from SSc lungs can indirectly inhibit cells which may be dependent on the IGF-II/IGF-IIE activity for the development and/or progression of SSc-associated pulmonary fibrosis, activation of growth factors, and so forth.

The binding proteins may be used to deliver an agent (e.g., any of a variety of cytotoxic and therapeutic drugs) to cells and tissues where IGF-II/IGF-IIE is present. Exemplary agents include a compound emitting radiation, molecules of plants, fungal, or bacterial origin, biological proteins, and mixtures thereof. The cytotoxic drugs can be intracellularly acting cytotoxic drugs, such as toxins short range radiation emitters, e.g., short range, high energy α-emitters.

To target IGF-II/IGF-IIE expressing cells, particularly lung fibroblasts or fibroblastic foci from SSc lungs, a prodrug system can be used. For example, a first binding protein is conjugated with a prodrug which is activated only when in close proximity with a prodrug activator. The prodrug activator is conjugated with a second binding protein, preferably one which binds to a non competing site on the target molecule. Whether two binding proteins bind to competing or non competing binding sites can be determined by conventional competitive binding assays. Exemplary drug prodrug pairs are described in Blakely et al., (1996) Cancer Research, 56:3287 3292.

The IGF-II/IGF-IIE binding proteins can be used directly in vivo to eliminate antigen-expressing cells via natural complement-dependent cytotoxicity (CDC) or antibody dependent cellular cytotoxicity (ADCC). The binding proteins described herein can include complement binding effector domain, such as the Fc portions from IgG1, -2, or -3 or corresponding portions of IgM which bind complement. In one embodiment, a population of target cells is ex vivo treated with a binding agent described herein and appropriate effector cells. The treatment can be supplemented by the addition of complement or serum containing complement. Further, phagocytosis of target cells coated with a binding protein described herein can be improved by binding of complement proteins. In another embodiment target, cells coated with the binding protein which includes a complement binding effector domain are lysed by complement.

Methods of administering IGF-II/IGF-IIE binding proteins are described in “Pharmaceutical Compositions.” Suitable dosages of the molecules used will depend on the age and weight of the subject and the particular drug used. The binding proteins can be used as competitive agents to inhibit or reduce an undesirable interaction, e.g., between a natural or pathological agent and the IGF-II/IGF-IIE.

The IGF-II/IGF-IIE binding protein can be used to deliver macro and micromolecules, e.g., a gene into the cell for gene therapy purposes into the endothelium or epithelium and target only those tissues expressing the IGF-II/IGF-IIE. The binding proteins may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short range radiation emitters, including, for example, short range, high energy α emitters, as described herein.

In the case of polypeptide toxins, recombinant nucleic acid techniques can be used to construct a nucleic acid that encodes the binding protein (e.g., antibody or antigen-binding fragment thereof) and the cytotoxin (or a polypeptide component thereof) as translational fusions. The recombinant nucleic acid is then expressed, e.g., in cells and the encoded fusion polypeptide isolated.

Alternatively, the IGF-II/IGF-IIE binding protein can be coupled to high energy radiation emitters, for example, a radioisotope, such as ¹³¹I, a γ-emitter, which, when localized at a site, results in a killing of several cell diameters. See, e.g., S.E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303 316 (Academic Press 1985). Other suitable radioisotopes include a emitters, such as ²¹²Bi, ²¹³Bi, and ²¹¹At, and b emitters, such as ¹⁸⁶Re and ⁹⁰Y. Moreover, ¹⁷⁷Lu may also be used as both an imaging and cytotoxic agent.

Radioimmunotherapy (RIT) using antibodies labeled with ¹³¹I, ⁹⁰Y, and ¹⁷⁷Lu is under intense clinical investigation. There are significant differences in the physical characteristics of these three nuclides and as a result, the choice of radionuclide is very critical in order to deliver maximum radiation dose to a tissue of interest. The higher beta energy particles of ⁹⁰Y may be good for bulky tumors. The relatively low energy beta particles of ¹³¹I are ideal, but in vivo dehalogenation of radioiodinated molecules is a major disadvantage for internalizing antibody. In contrast, ¹⁷⁷Lu has low energy beta particle with only 0.2-0 3 mm range and delivers much lower radiation dose to bone marrow compared to ⁹⁰Y. In addition, due to longer physical half-life (compared to ⁹⁰Y), the residence times are higher. As a result, higher activities (more mCi amounts) of ¹⁷⁷Lu labeled agents can be administered with comparatively less radiation dose to marrow. There have been several clinical studies investigating the use of ¹⁷⁷Lu labeled antibodies in the treatment of various cancers. (Mulligan T et al., 1995, Clin. Canc. Res. 1: 1447-1454; Meredith R F, et al., 1996, J. Nucl. Med. 37:1491-1496; Alvarez R D, et al., 1997, Gynecol. Oncol. 65: 94-101).

### Exemplary Diseases and Conditions

The IGF-II/IGF-IIE binding proteins described herein are useful to treat diseases or conditions in which IGF-II and/or IGF-IIE activity is implicated, e.g., a disease or condition described herein, or to treat one or more symptoms associated therewith. In some embodiments, the IGF-II/IGF-IIE binding protein (e.g., IGF-II/IGF-IIE binding IgG or Fab) inhibits IGF-II and/or IGF-IIE activity.

An example of such diseases and conditions includes systemic sclerosis-associated pulmonary fibrosis. A therapeutically effective amount of a IGF-II/IGF-IIE binding protein is administered to a subject having or suspected of having a disorder in which IGF-II/IGF-IIE activity is implicated, thereby treating (e.g., ameliorating or improving a symptom or feature of a disorder, slowing, stabilizing and/or halting disease progression) the disorder.

The IGF-II/IGF-IIE binding protein is administered in a therapeutically effective amount. A therapeutically effective amount of an IGF-II/IGF-IIE binding protein is the amount which is effective, upon single or multiple dose administration to a subject, in treating a subject, e.g., curing, alleviating, relieving or improving at least one symptom of a disorder in a subject to a degree beyond that expected in the absence of such treatment. A therapeutically effective amount of the composition may vary according to factors such as the disease state, age, sex, and weight of the individual, and the ability of the compound to elicit a desired response in the individual. A therapeutically effective amount is also one in which any toxic or detrimental effects of the composition is outweighed by the therapeutically beneficial effects.

A therapeutically effective amount can be administered, typically an amount of the compound which is effective, upon single or multiple dose administration to a subject, in treating a subject, e.g., curing, alleviating, relieving or improving at least one symptom of a disorder in a subject to a degree beyond that expected in the absence of such treatment. A therapeutically effective amount of the composition may vary according to factors such as the disease state, age, sex, and weight of the individual, and the ability of the compound to elicit a desired response in the individual. A therapeutically effective amount is also one in which any toxic or detrimental effects of the composition is outweighed by the therapeutically beneficial effects. A therapeutically effective dosage preferably modulates a measurable parameter, favorably, relative to untreated subjects. The ability of a compound to inhibit a measurable parameter can be evaluated in an animal model system predictive of efficacy in a human disorder.

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

### Systemic Sclerosis-Associated Pulmonary Fibrosis

Fibrosis is the formation or development of excess fibrous connective tissue in an organ or tissue as a reparative or reactive process, as opposed to a formation of fibrous tissue as a normal constituent of an organ or tissue. Pulmonary fibrosis involves scarring of the lung. Gradually, the air sacs of the lungs become replaced by fibrotic tissue. When the scar forms, the tissue becomes thicker causing an irreversible loss of the tissue's ability to transfer oxygen into the bloodstream.

Scleroderma is a chronic autoimmune disease characterized by hardening or sclerosis in the skin or other organs. Systemic sclerosis (SSc), the generalized and systemic type of the disease, can be fatal as a result of heart, kidney, lung, or intestinal damage. Pulmonary fibrosis in systemic sclerosis is associated with significant morbidity and mortality. At least one-third of patients with SSc have clinically significant pulmonary fibrosis, and lung function impairment is evident in up to 70% of patients with SSc. The 10-year survival rate from the time of presentation with pulmonary fibrosis in SSc approximates 70%, and many patients experience disabling progressive breathlessness. It is believed that chronic inflammation leads to progressive lung injury and incremental fibrosis.

The symptoms of pulmonary fibrosis include e.g., shortness of breath (particularly with exertion), a chronic dry and/or hacking cough, fatigue and weakness, discomfort in the chest, loss of appetite, and rapid weight loss. Untreated individuals develop complications that include emphysema, pulmonary infections, and cardiac disease.

Approximately five million people worldwide are affected by pulmonary fibrosis. In the United States, there are over 200,000 such patients. Typically, patients are in their forties and fifties when diagnosed. However, diagnoses have ranged from age seven to ages in the eighties.

The causes and risk factors of pulmonary fibrosis have been associated with e.g., autoimmune disorder, viral infection, genetic predisposition (e.g., a mutation in the SP—C protein), microscopic injury to the lung, inhaled environmental and/or occupational pollutants, cigarette smoking, diseases such as scleroderma, rheumatoid arthritis, lupus and sarcoidosis, certain medications, and therapeutic radiation.

Increased production of collagen and/or fibronectin is a characteristic of systemic sclerosis-associated pulmonary fibrosis. Fibroblastic foci (FF), sites of active collagen and/or fibronectin synthesis, are the pathologic hallmark of pulmonary fibrosis and the places where fibrotic responses are initiated and/or perpetuated in this severe disease.

Collagen is the main protein of connective tissue in animals and the most abundant protein in mammals, making up about 50% of the whole-body protein content. There are more than 28 types of collagen described in literature. Over 90% of the collagen in the body are of type I, II, III, and IV.

Fibronectin is a high-molecular-weight extracellular matrix glycoprotein containing about 5% carbohydrate that binds to membrane spanning receptor proteins called integrins and extracellular matrix components such as collagen, fibrin and heparan sulfate. There are several isoforms of fibronectin, all of which are the product of a single gene. The structure of these isoforms are made of three types of repeated internal regions called I, II and III which exhibit different lengths and presence or absence of disulfide bonds. Alternative splicing of the pre-mRNA leads to the combination of these three types of regions but also to a variable region.

Current treatments include e.g., certain anti-inflammatory drugs (e.g., steroids), cytotoxic drugs, immunosuppressive agents, collagen synthesis inhibitors, endothelin receptor antagonist and surgery. For example, high doses of oral corticosteroids (e.g., prednisone, 40 to 80 mg daily) are the usual treatment. Cytotoxic drugs such as cyclophosphamide and immunosuppressants such as azathioprine (cyclophosphamide is also an immunosuppressant) have also been used. Clinical experience with these drugs suggests that about 20 percent of patients will improve. The response to corticosteroids is better in patients with more inflammation and less fibrosis noted on lung biopsy. Collagen synthesis inhibitors such as Pirfenidone and endothelin receptor antagonists such as Bosentan may also be effective. Lung transplantation for highly selected patients with end-stage pulmonary fibrosis has been reported. In particular, cyclophosphamide or azathioprine can be used to treat SSc-associated pulmonary fibrosis. As further examples, pulses of cyclophosphamide, often together with a small dose of steroids; epoprostenol, bosentan or iloprost (e.g, aerolized iloprost) can be used.

Various lung function tests (also called pulmonary function tests) can be used to determine the cause of lung problems, evaluate a person's lung function and monitor the effectiveness of treatment for lung diseases. For example, spirometry measures how much and how quickly air can be moved out of lungs. The common lung function values measured with spirometry are: Forced vital capacity (FVC), which measures the amount of air a subject can exhale with force after the subject inhales as deeply as possible; Forced expiratory volume (FEV), which measures the amount of air a subject can exhale with force in one breath. The amount of air a subject exhales may be measured at 1 second (FEV1), 2 seconds (FEV2), or 3 seconds (FEV3). FEV1 divided by FVC can also be determined; Forced expiratory flow 25% to 75%, which measures the air flow halfway through an exhale (FVC); Peak expiratory flow (PEF), which measures how quickly a subject can exhale; Maximum voluntary ventilation (MVV), which measures the greatest amount of air a subject can breathe in and out during one minute; Slow vital capacity (SVC), which measures the amount of air a subject can slowly exhale after the subject inhales as deeply as possible; Total lung capacity (TLC), which measures the amount of air in a subject's lungs after the subject inhales as deeply as possible; Functional residual capacity (FRC), which measures the amount of air in a subject's lungs at the end of a normal exhaled breath; Expiratory reserve volume (ERV), which measures the difference between the amount of air in a subject's lungs after a normal exhale (FRC) and the amount after the subject exhales with force (RV). Gas diffusion tests measure the amount of oxygen and other gases that cross the lungs' air sacs (alveoli) per minute: Arterial blood gases, which determine the amount of oxygen and carbon dioxide in a subject's bloodstream; and Carbon monoxide diffusing capacity (also called transfer factor, or TF), which measures how well a subject's lungs transfer a small amount of carbon monoxide (CO) into the blood. Body plethysmography may be used to measure: Total lung capacity (TLC), which is the total amount of air a subject's lungs can hold. Residual volume (RV), which is the amount of air that remains in a subject's lungs after the subject exhales as completely as possible. Inhalation challenge tests are used to measure the response of a subject's airways to substances (allergens) that may be causing asthma or wheezing. Exercise stress tests evaluate the effect of exercise on lung function tests.

The disclosure provides methods of treating a symptom of systemic sclerosis-associated pulmonary fibrosis (e.g., reducing or eliminating fibroblastic foci, shortness of breath, chronic cough, fatigue and weakness, discomfort in the chest, loss of appetite, and/or rapid weight loss) or systemic sclerosis-associated pulmonary fibrosis, or increasing disease-free survival time in a subject previously diagnosed with systemic sclerosis-associated pulmonary fibrosis by administering an effective amount of an IGF-II/IGF-IIE binding protein (e.g., an anti-IGF-II/IGF-IIE IgG or Fab), e.g., an IGF-II/IGF-IIE binding protein described herein. In some embodiments, the IGF-II/IGF-IIE binding protein inhibits IGF-II/IGF-IIE activity.

In certain embodiments, the IGF-II/IGF-IIE binding protein is administered as a single agent treatment. In other embodiments, the IGF-II/IGF-IIE binding protein is administered in combination with an additional agent to treat SSc-associated pulmonary fibrosis.

Also provided are methods of preventing or reducing risk of developing systemic sclerosis-associated pulmonary fibrosis by administering an effective amount of an IGF-II/IGF-IIE binding protein to a subject at risk of developing systemic sclerosis-associated pulmonary fibrosis, thereby reducing the subject's risk of developing systemic sclerosis-associated pulmonary fibrosis. For example, the methods can be used to delay the development and/or slow the progression of SSc-associated pulmonary fibrosis or a symptom thereof (e.g., fibroblastic foci, shortness of breath, chronic cough, fatigue and weakness, discomfort in the chest, loss of appetite, rapid weight loss), e.g., in a subject who exhibits one or more symptoms of SSc-associated pulmonary fibrosis or is at risk of developing SSc-associated pulmonary fibrosis (e.g., the subject has been diagnosed with SSc or has a symptom or risk factor thereof, or a subject has one or more risk factors for SSc-associated pulmonary fibrosis, e.g., autoimmune disorder, viral infection, genetic predisposition (e.g., a mutation in the SP-C protein), microscopic injury to the lung, inhaled environmental and/or occupational pollutants, cigarette smoking, diseases such as scleroderma, rheumatoid arthritis, lupus and sarcoidosis, certain medications, and therapeutic radiation.). Predisposition to SSc includes familial predisposition for autoimmune disease; polymorphisms in COL1A2 and TGF-β1 may influence severity and development of the SSC; there is limited evidence implicating cytomegalovirus (CMV) as the original epitope of the immune reaction, and organic solvents and other chemical agents have been linked with scleroderma.

Guidance for determination of a therapeutically effective amount for treatment of systemic sclerosis-associated pulmonary fibrosis may be obtained by reference to in vivo models of the systemic sclerosis-associated pulmonary fibrosis to be treated. For example, the amount of an IGF-II/IGF-IIE binding protein that is a therapeutically effective amount in a rodent model of pulmonary fibrosis may be used to guide the selection of a dose that is a therapeutically effective amount. A number of rodent models of pulmonary fibrosis are available (see, e.g., Moore and Hogaboam et al. Am J Physiol Lung Cell Mol. Physiol. 294:L152-60 (2008)).

### Combination Therapies

The IGF-II/IGF-IIE binding proteins described herein, e.g., anti-IGF-II/IGF-IIE Fabs or IgGs, can be administered in combination with one or more of the other therapies for treating a disease or condition associated with IGF-II/IGF-IIE activity, e.g., a disease or condition described herein. For example, an IGF-II/IGF-IIE binding protein can be used therapeutically or prophylactically with surgery, an IGF-II inhibitor, e.g., a small molecule inhibitor, another anti-IGF-II/IGF-IIE Fab or IgG (e.g., another Fab or IgG described herein), another IGF-II inhibitor, a peptide inhibitor, or small molecule inhibitor. Examples of IGF-II inhibitors that can be used in combination therapy with an IGF-II/IGF-IIE binding protein described herein include anti-IGF-II antibodies that cross react with the IGF-I and IGF-II (see, e.g., WO2007118214, WO2007070432, EP1505075, US20060165695, WO2005028515, WO2005027970, WO2005018671) as well as anti-IGF-II antibodies that react only with IGF-II (see, e.g., WO2007118214).

One or more small-molecule IGF-II/IGF-IIE inhibitors can be used in combination with one or more IGF-II/IGF-IIE binding proteins described herein. For example, the combination can result in a lower dose of the small-molecule inhibitor being needed, such that side effects are reduced.

The IGF-II/IGF-IIE binding proteins described herein can be administered in combination with one or more current therapies for treating systemic sclerosis-associated pulmonary fibrosis, including, but not limited to: surgery. For example, proteins that inhibit IGF-II or that inhibit a downstream event of IGF-II/IGF-IIE activity can also be used in combination with another treatment for SSc-associated pulmonary fibrosis, such as surgery or administration of a second agent. For example, the second agent can include certain anti-inflammatory drugs (e.g., steroids), cytotoxic drugs, immunosuppressive agents, collagen synthesis inhibitors, endothelin receptor antagonist or surgery. For example, high doses of oral corticosteroids (e.g., prednisone, 40 to 80 mg daily) are the usual treatment. Cytotoxic drugs such as cyclophosphamide and immunosuppressants such as azathioprine (cyclophosphamide is also an immunosuppressant) have also been used. Collagen synthesis inhibitors such as Pirfenidone and endothelin receptor antagonists such as Bosentan may also be effective. Lung transplantation for highly selected patients with end-stage pulmonary fibrosis has been reported. In particular, cyclophosphamide or azathioprine can be used to treat SSc-associated pulmonary fibrosis. As further examples, pulses of cyclophosphamide, often together with a small dose of steroids; epoprostenol, bosentan or iloprost (e.g, aerolized iloprost) can be used.

The term “combination” refers to the use of the two or more agents or therapies to treat the same patient, wherein the use or action of the agents or therapies overlap in time. The agents or therapies can be administered at the same time (e.g., as a single formulation that is administered to a patient or as two separate formulations administered concurrently) or sequentially in any order. Sequential administrations are administrations that are given at different times. The time between administration of the one agent and another agent can be minutes, hours, days, or weeks. The use of an IGF-II/IGF-IIE binding protein described herein can also be used to reduce the dosage of another therapy, e.g., to reduce the side-effects associated with another agent that is being administered, e.g., to reduce the side-effects of an anti-VEGF antibody such as bevacizumab. Accordingly, a combination can include administering a second agent at a dosage at least 10, 20, 30, or 50% lower than would be used in the absence of the IGF-II/IGF-IIE binding protein.

The second agent or therapy can also be another agent for SSc-associated pulmonary fibrosisor therapy. Nonlimiting examples of another treatment for SSc-associated pulmonary fibrosis include, e.g., anti-inflammatory drugs e.g., steroids (e.g., corticosteroids (e.g., prednisone)), cytotoxic drugs (e.g., cyclophosphamide), immunosuppressants (e.g., cyclophosphamide or azathioprine), collagen synthesis inhibitors (e.g., Pirfenidone), endothelin receptor antagonists (e.g., Bosentan) and surgery (e.g., lung transplant), and other agents described herein.

A combination therapy can include administering an agent that reduces the side effects of other therapies. The agent can be an agent that reduces the side effects of SSc-associated pulmonary fibrosis treatments. For example, the agent can be a corticosteroid or cyclophosphamide.

### Diagnostic Uses

Proteins that bind to IGF-II/IGF-IIE and identified by the method described herein and/or detailed herein have in vitro and in vivo diagnostic utilities. The IGF-II/IGF-IIE binding proteins described herein (e.g., the proteins that bind and inhibit, or the proteins that bind but do not inhibit IGF-II/IGF-IIE) can be used, e.g., for in vivo imaging, e.g., during a course of treatment for a disease or condition in which IGF-II and/or IGF-IIE is active, e.g., a disease or condition described herein, or in diagnosing a disease or condition described herein.

In one aspect, the disclosure provides a diagnostic method for detecting the presence of IGF-II and/or IGF-IIE, in vitro or in vivo (e.g., in vivo imaging in a subject). The method can include localizing IGF-II and/or IGF-IIE within a subject or within a sample from a subject. With respect to sample evaluation, the method can include, for example: (i) contacting a sample with IGF-II/IGF-IIE binding protein; and (ii) detecting location of the IGF-II/IGF-IIE binding protein in the sample.

An IGF-II/IGF-IIE binding protein can also be used to determine the qualitative or quantitative level of expression of IGF-II and/or IGF-IIE in a sample. The method can also include contacting a reference sample (e.g., a control sample) with the binding protein, and determining a corresponding assessment of the reference sample. A change, e.g., a statistically significant change, in the formation of the complex in the sample or subject relative to the control sample or subject can be indicative of the presence of IGF-II and/or IGF-IIE in the sample. In one embodiment, the IGF-II/IGF-IIE binding protein does not cross react with another IGF protein, such as IGF-I. E.g, the binding protein binds to another IGF protein 5- to 10-fold less well (or even less well) than it binds to IGF-II/IGF-IIE. For example, the binding protein can bind to IGF-II/IGF-IIE with a KD of ˜10-50 pM, whereas it binds to IGF-I at ˜10 nM.

The IGF-II/IGF-IIE binding protein can be directly or indirectly labeled with a detectable substance to facilitate detection of the bound or unbound antibody. Suitable detectable substances include various enzymes, prosthetic groups, fluorescent materials, luminescent materials and radioactive materials.

Complex formation between the IGF-II/IGF-IIE binding protein and IGF-II and/or IGF-IIE can be detected by evaluating the binding protein bound to the IGF-II/IGF-IIE or unbound binding protein. Conventional detection assays can be used, e.g., an enzyme-linked immunosorbent assays (ELISA), a radioimmunoassay (RIA) or tissue immunohistochemistry. Further to labeling the IGF-II/IGF-IIE binding protein, the presence of IGF-II and/or IGF-IIE can be assayed in a sample by a competition immunoassay utilizing standards labeled with a detectable substance and an unlabeled IGF-II/IGF-IIE binding protein. In one example of this assay, the biological sample, the labeled standards, and the IGF-II/IGF-IIE binding protein are combined and the amount of labeled standard bound to the unlabeled binding protein is determined. The amount of IGF-II and/or IGF-IIE in the sample is inversely proportional to the amount of labeled standard bound to the IGF-II/IGF-IIE binding protein.

Fluorophore and chromophore labeled proteins can be prepared. Because antibodies and other proteins absorb light having wavelengths up to about 310 nm, the fluorescent moieties should be selected to have substantial absorption at wavelengths above 310 nm and preferably above 400 nm A variety of suitable fluorescers and chromophores are described by Stryer, 1968, Science 162:526 and Brand, L. et al., 1972, Annu. Rev. Biochem. 41:843 868. The proteins can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the protein can be used to detect the presence or localization of the IGF-II and/or IGF-IIE in a sample, e.g., using fluorescent microscopy (such as confocal or deconvolution microscopy).

Histological Analysis. Immunohistochemistry can be performed using the proteins described herein. For example, in the case of an antibody, the antibody can be synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation for binding, the preparation is washed to remove unbound antibody. The preparation is then analyzed, e.g., using microscopy, to identify if the antibody bound to the preparation.

Of course, the antibody (or other polypeptide or peptide) can be unlabeled at the time of binding. After binding and washing, the antibody is labeled in order to render it detectable.

Protein Arrays. The IGF-II/IGF-IIE binding protein can also be immobilized on a protein array. The protein array can be used as a diagnostic tool, e.g., to screen medical samples (such as isolated cells, blood, sera, biopsies, and the like). Of course, the protein array can also include other binding proteins, e.g., that bind to IGF-II and/or IGF-IIE or to other target molecules.

Methods of producing polypeptide arrays are described, e.g., in De Wildt et al., 2000, Nat. Biotechnol. 18:989-994; Lueking et al., 1999, Anal. Biochem. 270:103-111; Ge, 2000, Nucleic Acids Res. 28, e3, I-VH; MacBeath and Schreiber, 2000, Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

For example, the array can be an array of antibodies, e.g., as described in De Wildt, supra. Cells that produce the proteins can be grown on a filter in an arrayed format. Polypeptide production is induced, and the expressed polypeptides are immobilized to the filter at the location of the cell. A protein array can be contacted with a labeled target to determine the extent of binding of the target to each immobilized polypeptide. Information about the extent of binding at each address of the array can be stored as a profile, e.g., in a computer database. The protein array can be produced in replicates and used to compare binding profiles, e.g., of a target and a non-target.

FACS (Fluorescence Activated Cell Sorting). The IGF-II/IGF-IIE binding protein can be used to label cells, e.g., cells in a sample (e.g., a patient sample). The binding protein is also attached (or attachable) to a fluorescent compound. The cells can then be sorted using fluorescence activated cell sorter (e.g., using a sorter available from Becton Dickinson Immunocytometry Systems, San Jose Calif.; see also U.S. Pat. Nos. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample.

The sorter can also deflect the cell and separate cells bound by the binding protein from those cells not bound by the binding protein. The separated cells can be cultured and/or characterized.

In vivo Imaging. Also featured is a method for detecting the presence of an IGF-II and/or IGF-IIE expressing tissues in vivo. The method includes (i) administering to a subject (e.g., a patient having, e.g., systemic sclerosis-associated pulmonary fibrosis an anti-IGF-II/IGF-IIE antibody, conjugated to a detectable marker; (ii) exposing the subject to a means for detecting said detectable marker to the IGF-II and/or IGF-IIE expressing tissues or cells. For example, the subject is imaged, e.g., by NMR or other tomographic means.

Examples of labels useful for diagnostic imaging include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short range radiation emitters, such as isotopes detectable by short range detector probes can also be employed. The protein can be labeled with such reagents; for example, see Wensel and Meares, 1983, Radioimmunoimaging and Radioimmunotherapy, Elsevier, New York for techniques relating to the radiolabeling of antibodies and D. Colcher et al., 1986, Meth. Enzymol. 121: 802 816.

The binding protein can be labeled with a radioactive isotope (such as ¹⁴C, ³H, ³⁵S, ¹²⁵I, ³²P, ¹³¹I).

A radiolabeled binding protein can be used for diagnostic tests, e.g., an in vitro assay. The specific activity of a isotopically-labeled binding protein depends upon the half life, the isotopic purity of the radioactive label, and how the label is incorporated into the antibody.

In the case of a radiolabeled binding protein, the binding protein is administered to the patient, is localized to cells bearing the antigen with which the binding protein reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65 85 (Academic Press 1985). Alternatively, a positron emission transaxial tomography scanner, such as designated Pet VI located at Brookhaven National Laboratory, can be used where the radiolabel emits positrons (e.g. ¹¹C, ¹⁸F, ¹⁵O, and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., Fe⁺³, Mn⁺², Gd⁺³). Other agents can be in the form of particles, e.g., less than 10 mm to about 10 nM in diameter). Particles can have ferromagnetic, antiferromagnetic, or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like.

The IGF-II/IGF-IIE binding protein can also be labeled with an indicating group containing of the NMR active ¹⁹F atom, or a plurality of such atoms inasmuch as (i) substantially all of naturally abundant fluorine atoms are the ¹⁹F isotope and, thus, substantially all fluorine containing compounds are NMR active; (ii) many chemically active polyfluorinated compounds such as trifluoracetic anhydride are commercially available at relatively low cost; and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett, 1982, Sci. Am. 246:78 88 to locate and image tissues expressing IGF-II and/or IGF-IIE.

### EXEMPLIFICATION

The present invention is further illustrated by the following examples which should not be construed as limiting in any way. The contents of all references, pending patent applications and published patents, cited throughout this application are hereby expressly incorporated by reference.

### Example 1

### Selection and Screening of Anti-IGF-II/IGF-IIE Antibodies from Libraries

Phage displaying Fabs were first placed in contact with magnetic streptavidin beads to deplete those which might bind to streptavidin beads. Non-binding phage were then placed in contact with biotinylated IGF-IIE (amino acids 1-104) immobilized on streptavidin magnetic beads. Unbound phage were washed away and beads with bound phage placed with E. coli cells for propagation of phage. Propagated phage were placed in contact with magnetic streptavidin beads and with biotinylated IGF-I immobilized on streptavidin beads for depletion purposes. Unbound phage were placed in contact with biotinylated IGF-IIE (amino acids 1-104) immobilized on streptavidin magnetic beads as before. Unbound phage were washed away and the whole process repeated for one more cycle. Gene III removal was then performed on propagated output phage. sFab ELISAs were then performed using IGF-IIE (amino acids 1-104), IGF-II (amino acids 1-67), IGF-I and streptavidin as targets. Those sFab binding IGF-IIE and IGF-II, but not IGF-I or streptavidin were then pursued. sFabs were screened for inhibition on IGF-II or IGF-IIE stimulated BA/F3 cell proliferation using the following materials and methods:

Cell Culture and Materials:

BA/F3 cells were cultured in complete medium (90% RPMI 1640+10% FBS+10 ng/ml IL-3+2 mM L-Alanyl-Glutamine+1×Pen/Strep). Cells at passage 6 to 15 were used for the high throughput cell proliferation assay. IGF-II (67aa), IGF-IIE (104aa) were at 10 μg/ml in PBS, kept at −70° C. IL-4 was purchased from R&D system, Cat#: 404-ML, and anti-IGF-II antibody was purchased from R&D system, Cat#: MAB292. 34 sFabs from batch 1 and batch 2 in a median scale purification, all in PBS, were screened.

Screening Procedure:

IGF-II was prepared at concentration of 400 ng/ml; IGF-IIE at 800 ng/ml; sFab at 200 μg/ml in PBS. 25 μl of IGF-II or IGF-IIE was preincubated with 25 μl of sFab in 96-well plate, triplicate for each sFab, 30 min at room temperature in a total volume of 50 μl/well. The cells were prepared as follows:
Harvest BA/F3 cells by centrifuging at 1100 rpm for 5 min.
Remove supernatant, and resuspend cells in 20 ml of PBS.
Count cell density by mixing 10 μl of cell suspension with 10 μl of trypan blue solution and loading 10 μl to a hemocytometer to count cell density and viability.
Spin down cells from PBS solution at 1100 rpm for 5 min.
Remove supernatant, and resuspend cells in culture medium without IL-3 at 2×10⁶ cells/ml.
Add 25 μl of cell suspension to each well of the prepared 96-well plate to make the final cell density at 5×10⁴ cells/well.
Add 25 μl of IL-4 at 200 ng/ml in IL-3 free medium to each well to make the final con. 50 ng/ml.
The total volume is 100 μl per well. Neutralizing antibody from R&D at 50 μg/ml as positive control; no treatment as negative control. The final concentration of sFab: 50 μg/ml (1 μM)
Incubate the plate at 37° C., 5% CO₂ for 72 h.

The MTS assay was performed as follows:
Add 20 μl of CellTiter 96 Aqueous One Solution Reagent to each well
Incubate at 37° C., 5% CO₂ for additional 4 h.
Read plate for absorbance at a microplate spectrophotometer at wavelength 490 nm

There were 2 Fabs from the first batch and 6 Fabs from the second batch that showed significant inhibitory effect on both IGF-II and IGF-IIE stimulated BA/F3 cell proliferation. These 8 Fabs, M0068-E03, M0072-006, M0064-F02, M0072-G06, M0072-E03, M0070 H08, M0064-E04 and M0063-F02 were further evaluated for IC50 determination.

### Example 2

### IC50 Determination of Anti-IGF-IUIGF-IIE Fabs

The IC50 values of the 8 sFabs for inhibition on IGF-II or IGF-IIE stimulated BA/F3 cell proliferation were determined as follows:

Cell Culture and Materials:

BA/F3 cells were cultured in complete medium (90% RPMI 1640+10% FBS+10 ng/ml IL-3+2 mM L-Alanyl-Glutamine+1×Pen/Strep). Cells at passage 24 were used for cell proliferation assay. IGF-II (67aa), IGF-IIE (104aa) were at 10 μg/ml in PBS and kept at −70° C. IL-4 was purchased from R&D system, Cat#: 404-ML, and anti-IGF-II antibody was purchased from R&D system, Cat#: MAB292. The 8 sFabs from Example 1 were subjected to medium scale purification, in PBS.

Procedure:
Prepare IGF-II at concentration of 400 ng/ml; IGF-IIE at 800 ng/ml; sFab at 200 μg/ml in PBS.
Pre-incubate 25 μl of IGF-II or IGF-IIE with 25 μl of 1:2 serially diluted sFab in a 96-well plate (50 μg/ml down to 0), triplicate for each dose of each sFab, incubate for 30 min at room temperature in a total volume of 50 μl/well.
Prepare cells:
   Harvest BA/F3 cells by centrifuging at 1100 rpm for 5 min.
   Remove supernatant, and resuspend cells in 20 ml of PBS.
   Count cell density by mixing 10 μl of cell suspension with 10 μl of trypan blue solution and loading 10 μl to a hemocytometer to count cell density and viability.
   Spin down cells from PBS solution at 1100 rpm for 5 min.
   Remove supernatant, and resuspend cells in culture medium without IL-3 at 2×10⁶ cells/ml.
   Add 25 μl of cell suspension to each well of the prepared 96-well plate to make the final cell density at 5×10⁴ cells/well.
   Add 25 μl of IL-4 at 200 ng/ml in IL-3 free medium to each well to make the final con. 50 ng/ml.
   The total volume is 100 μl per well.
   Incubate the plate at 37° C., 5% CO₂ for 72 h.

MTS Assay:
Add 20 μl of CellTiter 96 Aqueous One Solution Reagent to each well
Incubate at 37° C., 5% CO₂ for additional 4 h.
Read plate for absorbance at a microplate spectrophotometer at wavelength 490 nm

An inhibitory effect of anti-IGF-II/IIE sFabs was observed for 6 out of 8 Fabs for cell proliferation in a dose dependent manner with IC50 values shown in Table 2. Fab 72E03 showed some inhibition, but not as significant as others in IGF-IIE stimulated cell proliferation. The IC50 value of 72E03 was not calculated due to low potency. Fab 70H08 did not show significant inhibition.

TABLE 2
IC50 Values of the 6 Fabs which showed inhibitory effect on both
IGF-II and IGF-IIE stimulated cell proliferation.

| IC50 | M0072- | M0063- | M0064- | M0064- | M0068- | M0072- |
|---|---|---|---|---|---|---|
| μg/ml | G06 | F02 | F02 | E04 | E03 | C06 |
| IGF-II | 8.7 ± | 5.56 ± | 0.68 ± | 3.04 ± | 2.27 ± | 5.12 ± |
| | 7.17 | 2.61 | 0.31 | 1.89 | 0.71 | 3.25 |
| IGF-IIE | 2.5 ± | 2.04 ± | 3.12 ± | 5.46 ± | 5.74 ± | 2.3 ± |
| | 0.87 | 0.5 | 1.44 | 2.9 | 3.43 | 0.59 |

An inhibitory effect of most sFabs at 50 μg/ml on IGF-I stimulated cell proliferation was observed. Neu-IGF-I antibody showed potent inhibition; Neu-IGF-II antibody and all other sFabs except for 70H08 also showed about 30% inhibition.

In conclusion, 6 sFabs (M0072-G06, M0063-F02, M0064-F02, M0064-E0, M0068-E03 and M0072-006) demonstrated significant inhibitory effect on both IGF-II and IGF-IIE stimulated BaF3 cell proliferation. Further, all soluble Fabs except M0070-H08 at 50 μg/ml showed more or less inhibition (−30%) on IGF-I stimulated cell proliferation.

### Example 3

### DNA and Amino Acid Sequences of Anti-IGF-II/IGF-IIE Fabs

Exemplary Fabs that bind to both human IGF-II/IGF-IIE were identified as described above and designated as: M0033-E05, M0063-F02, M0064-E04, M0064-F02, M0068-E03, M0070-H08, M0072-006, M0072-E03, and M0072-G06. The DNA sequences of these Fab light chain variable regions (LV), light chain constant regions (LC), heavy chain variable regions (HV) and heavy chain constant regions (HC) are shown in Table 3. DNA sequences encoding the CDR regions are shown in bold.

TABLE 3
DNA sequences of anti-IGF-II/IGF-IIE Fabs

>M0063-F02(R0032-A01) LV
CAAGACATCCAGATGACCCAGTCTCCATCCTCCCTGTCTGCATCTGTAGGAGACAGAGTCACCATCACTTG
CCGGGCAAGTCAGCCCATTAACACATATTTAAATTGGTATCAGCAGAGACCAGGGAAAGCCCCTAGGATCC
TGATCTATACTTCATCCACTTTGCAAAGTGGGGTCCCATCAAGGTTCAGTGGCAGTGGATCTGGGACAGAT
TTCACTCTCACCATCAGCAGTGTGCAACCTGAAGATTTTGCAACTTACTACTGTCAACAGAGTTACAGTTT
CCCTCTCACTTTCGGCGGAGGGACCAAGGTGGAGATCAAA
>M0063-F02(R0032-A01) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0063-F02(R0032-A01) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTAAGTACGAGATGGATTGGGTTCGCCAAGCTCCTGGTAAAGGTTTGGAGTGGG
TTTCTGTTATCTCTTCTTCTGGTGGCGGTACTATTTATGCTGACTCCGTTAAAGGTCGCTTCACTATCTCT
AGAGACAACTCTAAGAATACTCTCTACTTGCAGATGAACAGCTTAAGGGCTGAGGACACCGCCATGTATTA
CTGTGCGAGAGGCCGGACCCTATACGGAGGTGCTTTTGATATCTGGGGCCAAGGGACAATGGTCACCGTCT
CAAGC
>M0063-F02(R0032-A01) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCG
>M0064-E04(R0032-A03) LV
CAAGACATCCAGATGACCCAGTCTCCATCCTCCCTGTCTGCATCTGTAGGAGACAGAGTCACCATCACTTG
CCAGGCGAGTCACGACATTAGCAACTATTTAAATTGGTATCAGCAGAAACCAGGGAAAGCCCCTAAGCTCC
TGATTTATGCTGCATCCCGTTTGCAAAGTGGGGTCCCATCAAGGTTCAGTGGCGGTGGATCTGGGACAGAT
TTCAGTCTCACCATCAGCAGTCTGCAAGCTGAAGATTTTGCAACTTATTACTGTCAACAGAGTTACAGTTT
CCCTCGAACTTTTGGCCAGGGGACCAACCTGGAGATCAAA
>M0064-E04(R0032-A03) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGTAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0064-E04(R0032-A03) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTGTTTACGATATGAATTGGGTTCGCCAAGCTCCTGGTAAAGGTTTGGAGTGGG
TTTCTTCTATCTCTTCTTCTGGTGGCGGTACTCTTTATGCTGACTCCGTTAAAGGTCGCTTCACTATCTCT
AGAGACAACTCTAAGAATACTCTCTACTTGCAGATGAACAGCTTAAGGGCTGAGGACACGGCTGTGTATTA
CTGTGCGAGAGACTCTGATACCAGTTCTTATTACTGGTACTACGATCTCTGGGGCCGCGGCACCCTGGTCA
CCGTCTCAAGC
>M0064-E04(R0032-A03) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCGTGATGA
>M0033-E05(R0032-A05) LV
CAAGACATCCAGATGACCCAGTCTCCATCCTCACTGTCTGCATCTGTTGGAGACAGAGTCACCATCAGTTG
TCGGGCAAGTCAGGGCATTACCAATTATTTAGTCTGGTTTCAGCAGAAACCAGGGAAAGCCCCTAGGCTCC
TGATCTATGATGCCTCCACTTTGGAAAGTGGGGTCCCATCAAGGTTCAGCGGCAGTGGATCTGGGACAGAT
TTCACTCTCACCATCAGCAGCCTGCAGCCTGAAGATTTTGCAACTTACTATTGTCAACAGGCTGACGGTTT
CCCTCTCACTTTCGGCGAGGGGACCAAGGTGGAGATGAAA
>M0033-E05(R0032-A05) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0033-E05 (R0032-A05) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTCATTACTCTATGTGGTGGGTTCGCCAAGCTCCTGGTAAAGGTTTGGAGTGGG
TTTCTTATATCGGTCCTTCTGGTGGCCATACTCGTTATGCTGACTCCGTTAAAGGTCGCTTCACTATCTCT
AGAGACAACTCTAAGAATACTCTCTACTTGCAGATGAACAGCTTAAGGGCTGAGGACACGGCCGTGTATTA
CTGTGCTAGAGGGCTATATTATTATGATAGTAGTAGCGTGACTCATGCCTTTGATCTCTGGGGCCAAGGGA
CAATGGTCACCGTCTCAAGC
>M0033-E05(R0032-A05) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCGTGATGA
>M0070-H08(R0032-A07) LV
CAAGACATCCAGATGACCCAGTCTCCGTCCTCCCTGTCTGCATCTGCAGGAGACAGAGTCACCATCACTTG
CCGGGCAAGTCAGAGCATTAGCAGTTATTTAAATTGGTATCAGCAGAAACCAGGAAAAGCCCCTAACCTCC
TGATCTATACTACATCCAATTTACAAGGTGGGGTCCCATCAAGGTTCAGTGGCAGTGGATCTGGGACAGAT
TTCACTCTCACCATCAGCAGTCTGCAACCTGAAGATTTTGCAACTTACTACTGTCAACAGGGTTACAGTTT
CCCTCTCACTTTCGGCGGAGGGACCAAGGTAGAGATCAAA
>M0070-H08(R0032-A07) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAACTCTACGCCTGCGAAGTCACCCATCAGGGCCTGAG
CTCGCCCGTCACAAAGAGCTTCAACAGGGGAGAGTGTTAATAA
>M0070-H08(R0032-A07) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTCGTTACTGGATGATTTGGGTTCGCCAAGCTCCTGGTAAAGGTTTGGAGTGGG
TTTCTTCTATCCGTTCTTCTGGCGAGACTAAGTATGCTGACTCCGTTAAAGGTCGCTTCACTATCTCTAGA
GACAACTCTAAGAATACTCTCTACTTGCAGATGAACAGCTTAAGGGCTGAGGACACGGCCGTGTATTACTG
TGCGAGAGGCCCTTTAAGCGATTACTATGATAGTAGTGGTTATTACTTTGATGCTTTTGATATCTGGGGCC
AAGGGACAATGGTCACCGTCTCAAGC
>M0070-H08(R0032-A07) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCG
>M0072-C06(R0032-A09) LV
CAAGACATCCAGATGACCCAGTCTCCATCCTCCCTGTCTGCATCTGTAGGAGACAGAGTCACCATCACTTG
CCGGGCAAGTCAGAGTATTCGCAACTATTTAAATTGGTATCAGCAGAAACCAGGGAAAGCCCCTAAACTCC
TGATCTATGCTGCATCCAAGTTGGAAGACGGGGTCCCATCAAGATTCAGTGGCAGTGGAACTGGGACAGAT
TTCACTCTCACCATCAGAAGTCTGCAACCTGAAGATTTTGCAAGTTATTTCTGTCAACAGAGCTACTCTAG
TCCAGGGATCACTTTCGGCCCTGGGACCAAGGTGGAGATCAAA
>M0072-C06(R0032-A09) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0072-C06(R0032-A09) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTGCTTACATTATGACTTGGGTTCGCCAAGCTCCTGGTAAAGGTTTGGAGTGGG
TTTCTTCTATCTCTCCTTCTGGTGGCTATACTGTTTATGCTGACTCCGTTAAAGGTCGCTTCACTATCTCT
AGAGACAACTCTAAGAATACTCTCTACTTGCAGATGAACAGCTTAAGGGCTGAGGACACGGCCGTATATTA
CTGTGCGAGAGACTCGGGGTTCGGGGACCCCTTTGACTACTGGGGCCAAGGGACAATGGTCACCGTCTCAA
GC
>M0072-C06(R0032-A09) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCGTGATGA
>M0064-F02(R0032-A11) LV
CAAGACATCCAGATGACCCAGTCTCCATCCTCCCTGTCTGCATCTGTAGGAGACAGAGTCACCATCACTTG
CCGGGCAAGTCAGAGCATTAGCAATTACTTGAATTGGTATCAACAGAAACCAGGTAAAGCCCCTAAGCTCC
TGATCTATACTGCATCCACTTTGCAGAGTGGGGTCCCATCAAGGTTCAGTGGCAGTGCATCTGGGACAGAC
TTCACTCTCACCATCAACAGTCTGCAACCTGAAGATTTTGCTACTTACTCCTGTCAACAGAGTTACAATTC
CCCCTGGACGTTCGGCCAAGGGACCAAGGTGGAAATCAAA
>M0064-F02(R0032-A11) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0064-F02(R0032-A11) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTAATTACATTATGTGGTGGGTTCGCCAAGCTCCTGGTAAAGGTTTGGAGTGGG
TTTCTGTTATCTCTTCTTCTGGTGGCATGACTCGTTATGCTGACTCCGTTAAAGGTCGCTTCACTATCTCT
AGAGACAACTCTAAGAATACTCTCTACTTGCAGATGAACAGCTTAAGGGCTGAGGACACGGCCGTGTATTA
CTGTGCGAGAGATAACGGTGACTACGTAGGCGAAAAAGGTTTTGATATCTGGGGCCAAGGGACAATGGTCA
CCGTCTCAAGC
>M0064-F02(R0032-A11) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCGTGATGA
>M0068-E03(R0032-C01) LV
CAAGACATCCAGATGACCCAGTCTCCATCCTCCCTGTCTGCATCTGTAGGAGACAGAGTCACCATCACTTG
CCGGGCAAGTCAGAGCATTAACACTTATTTAAATTGGTATCAGCAGAAACCAGGGAAAGCCCCTAAGGTCC
TGATCCATGCTGCATCCACTTTGGAAAGTGGGGTGCCATCAAGGTTCAGTGGCAGTGGATCTGCGACAGAA
TTCACTCTCACCATCAGCAGTCTGCAACCTGAAGATTTTGCAACTTTCTACTGTCAACAGAGTTACAGTGT
GCCCTTCACTTTCGGCCCTGGGACCAGACTGTCTAGCAAA
>M0068-E03(R0032-C01) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0068-E03(R0032-C01) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTGAGTACGTTATGGCTTGGGTTCGCCAAGCTCCTGGTAAAGGTTTGGAGTGGG
TTTCTTCTATCGTTTCTTCTGGTGGCTATACTAAGTATGCTGACTCCGTTAAAGGTCGCTTCACTATCTCT
AGAGACAACTCTAAGAATACTCTCTACTTGCAGATGAACAGCTTAAGGGCTGAGGACACGGCCGTGTATTA
CTGTGCAAAAGATATGACTTACAGTGGGGATGCTTTTGATGTCTGGGGCCAAGGGACAATGGTCACCGTCT
CAAGC
>M0068-E03(R0032-C01) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCGTGATGA
>M0072-E03(R0032-C03) LV
CAAGACATCCAGATGACCCAGTCTCCTTCCACCCTGTCTGCATCTGTAGGAGACAGAGTCACCATCACTTG
CCGGGCCAGTCAGAGTATTAGTAGCTGGTTGGCCTGGTATCAGCAGAAACCAGGGAAAGCCCCTAAGCTCC
TGATCTATAAGGCGTCTAGTTTAGAAAGTGGGGTCCCATCAAGGTTCAGCGGCAGTGGATCTGGGACAGAA
TTCACTCTCACCATCAGCAGCCTGCAGCCTGATGATTTTGCAACTTATTACTGCCAACAGTATAATAGTTA
TCCGTGGACGTTCGGCCAAGGGACCAAGGTGGAAATCAAA
>M0072-E03(R0032-C03) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0072-E03(R0032-C03) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTGAGTACGAGATGTCTTGGGTTCGCCAAGCTCCTGGTAAAGGTTTGGAGTGGG
TTTCTTCTATCTATTCTTCTGGTGGCTGGACTAAGTATACTGACTCCGTTAAAGGTCGCTTCACTATCTCT
AGAGACAACTCTAAGAATACTCTCTACTTGCAGATGAACAGCTTAAGGGCTGAGGACACGGCCGTGTATTA
CTGTGCGAAAGGTGTACACTATGATAGTAGTGGCCTTCCTATTGACTGGTACTTCGATCTCTGGGGCCGTG
GCACCCTGGTCACCGTCTCAAGC
>M0072-E03(R0032-C03) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCG
>M0072-G06(R0032-C05) LV
CAAGACATCCAGATGACCCAGTCTCCTTCCACCCTGTCTGCATCTGTAGGAGACAGAGTCACCATCACTTG
CCGGGCCAGTCAGACAATTAGTAGCTGGCTGGCCTGGTATCAGCAGAAACCAGGGAAAGCCCCTAAGTTGA
TGATCTATAAGGCGGCTAGTTTAGGAAGTGAGGTCCCATCAAGGTTCAGCGGCAGTGGATCTGGGACAGAG
TTCACTCTCACCATCAGCAGCCTGCAGCCTGAAGATTTTTCAACTTACTACTGCCAACAGTACAAAACTTA
TCCCGTCACTTTTGGCCAGGGGACCAGGCTGGAGATCAAA
>M0072-G06(R0032-C05) LC
CGAACTGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTTAATAA
>M0072-G06(R0032-C05) HV
GAAGTTCAATTGTTAGAGTCTGGTGGCGGTCTTGTTCAGCCTGGTGGTTCTTTACGTCTTTCTTGCGCTGC
TTCCGGATTCACTTTCTCTGAGTACGTTATGTGGTGGGTTCGCCAAGCTCCTGGTAAAGGTTTGGAGTGGG
TTTCTGTTATCTCTCCTTCTGGTGGCTATACTGTTTATGCTGACTCCGTTAAAGGTCGCTTCACTATCTCT
AGAGACAACTCTAAGAATACTCTCTACTTGCAGATGAACAGTTTAAGGGCTGAGGACACGGCCGTGTATTA
CTGTGCGAGAGATCGGGGGGGAGCTACTACCCTTGACTACTGGGGCCAGGGAACCCTGGTCACCGTCTCAA
GC
>M0072-G06(R0032-C05) HC
GCCTCCACCAAGGGCCCATCGGTCTTCCCGCTAGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCA
GCGGCGTCCACACCTTCCCGGCTGTCCTACAGTCTAGCGGACTCTACTCCCTCAGCAGCGTAGTGACCGTG
CCCTCTTCTAGCTTGGGCACCCAGACCTACATCTGCAACGTGAATCACAAGCCCAGCAACACCAAGGTGGA
CAAGAAAGTTGAGCCCAAATCTTGTGCGGCCGCACATCATCATCACCATCACGGGGCCGCAGAACAAAAAC
TCATCTCAGAAGAGGATCTGAATGGGGCCGCAGAGGCTAGTTCTGCTAGTAACGCGTGATGA

The amino acid sequences of exemplary Fab LV, LC, HV and HC regions that bind to and inhibit human IGF-II and IGF-IIE, the DNA sequence of which are provided in Table 3, are shown in Table 4. CDR regions are shown in bold.

TABLE 4
Amino Acid sequences of anti-IGF-II/IGF-IIE Fabs

>M0063-F02-R0032-A01-LV
QDIQMTQSPSSLSASVGDRVTITCRASQPINTYLNWYQQRPGKAPRILIYTSSTLQSGVPSRFSGSGSGTD
FTLTISSVQPEDFATYYCQQSYSFPLTFGGGTKVEIK
>M0063-F02-R0032-A01-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0063-F02-R0032-A01-HV
EVQLLESGGGLVQPGGSLRLSCAASGFTFSKYEMDWVRQAPGKGLEWVSVISSSGGGTIYADSVKGRFTIS
RDNSKNTLYLQMNSLRAEDTAMYYCARGRTLYGGAFDIWGQGTMVTVSS
>M0063-F02-R0032-A01-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0064-E04-R0032-A03-LV
QDIQMTQSPSSLSASVGDRVTITCQASHDISNYLNWYQQKPGKAPKLLIYAASRLQSGVPSRFSGGGSGTD
FSLTISSLQAEDFATYYCQQSYSFPRTFGQGTNLEIK
>M0064-E04-R0032-A03-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0064-E04-R0032-A03-HV
EVQLLESGGGLVQPGGSLRLSCAASGFTFSVYDMNWVRQAPGKGLEWVSSISSSGGGTLYADSVKGRFTIS
RDNSKNTLYLQMNSLRAEDTAVYYCARDSDTSSYYWYYDLWGRGTLVTVSS
>M0064-E04-R0032-A03-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0033-E05-R0032-A05-LV
QDIQMTQSPSSLSASVGDRVTISCRASQGITNYLVWFQQKPGKAPRLLIYDASTLESGVPSRFSGSGSGTD
FTLTISSLQPEDFATYYCQQADGFPLTFGEGTKVEMK
>M0033-E05-R0032-A05-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0033-E05-R0032-A05-HV
EVQLLESGGGLVQPGGSLRLSCAASGFTFSHYSMWWVRQAPGKGLEWVSYIGPSGGHTRYADSVKGRFTIS
RDNSKNTLYLQMNSLRAEDTAVYYCARGLYYYDSSSVTHAFDLWGQGTMVTVSS
>M0033-E05-R0032-A05-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0070-H08-R0032-A07-LV
QDIQMTQSPSSLSASAGDRVTITCRASQSISSYLNWYQQKPGKAPNLLIYTTSNLQGGVPSRFSGSGSGTD
FTLTISSLQPEDFATYYCQQGYSFPLTFGGGTKVEIK
>M0070-H08-R0032-A07-LC
RTVAAPSVFIFPPSDEQLKSGTASVVCLLNNFYPREAKVQWKVDNALQSGNSQESVTEQDSKDSTYSLSST
LTLSKADYEKHKLYACEVTHQGLSSPVTKSFNRGEC..
>M0070-H08-R0032-A07-HV
EVQLLESGGGLVQPGGSLRLSCAASGFTFSRYWMIWVRQAPGKGLEWVSSIRSSGETKYADSVKGRFTISR
DNSKNTLYLQMNSLRAEDTAVYYCARGPLSDYYDSSGYYFDAFDIWGQGTMVTVSS
>M0070-H08-R0032-A07-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0072-C06-R0032-A09-LV
QDIQMTQSPSSLSASVGDRVTITCRASQSIRNYLNWYQQKPGKAPKLLIYAASKLEDGVPSRFSGSGTGTD
FTLTIRSLQPEDFASYFCQQSYSSPGITFGPGTKVEIK
>M0072-C06-R0032-A09-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0072-C06-R0032-A09-HV
EVQLLESGGGLVQPGGSLRLSCAASGFTFSAYIMTWVRQAPGKGLEWVSSISPSGGYTVYADSVKGRFTIS
RDNSKNTLYLQMNSLRAEDTAVYYCARDSGFGDPFDYWGQGTMVTVSS
>M0072-C06-R0032-A09-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0064-F02-R0032-A11-LV
QDIQMTQSPSSLSASVGDRVTITCRASQSISNYLNWYQQKPGKAPKLLIYTASTLQSGVPSRFSGSASGTD
FTLTINSLQPEDFATYSCQQSYNSPWTFGQGTKVEIK
>M0064-F02-R0032-A11-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0064-F02-R0032-A11-HV
EVQLLESGGGLVQPGGSLRLSCAASGFTFSNYIMWWVRQAPGKGLEWVSVISSSGGMTRYADSVKGRFTIS
RDNSKNTLYLQMNSLRAEDTAVYYCARDNGDYVGEKGFDIWGQGTMVTVSS
>M0064-F02-R0032-A11-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0068-E03-R0032-C01-LV
QDIQMTQSPSSLSASVGDRVTITCRASQSINTYLNWYQQKPGKAPKVLIHAASTLESGVPSRFSGSGSATE
FTLTISSLQPEDFATFYCQQSYSVPFTFGPGTRLSSK
>M0068-E03-R0032-C01-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0068-E03-R0032-C01-HV
EVQLLESGGGLVQPGGSLRLSCAASGFTFSEYVMAWVRQAPGKGLEWVSSIVSSGGYTKYADSVKGRFTIS
RDNSKNTLYLQMNSLRAEDTAVYYCAKDMTYSGDAFDVWGQGTMVTVSS
>M0068-E03-R0032-C01-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0072-E03-R0032-C03-LV
QDIQMTQSPSTLSASVGDRVTITCRASQSISSWLAWYQQKPGKAPKLLIYKASSLESGVPSRFSGSGSGTE
FTLTISSLQPDDFATYYCQQYNSYPWTFGQGTKVEIK
>M0072-E03-R0032-C03-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0072-E03-R0032-C03-HV
EVQLLESGGGLVQPGGSLRLSCAASGFTFSEYEMSWVRQAPGKGLEWVSSIYSSGGWTKYTDSVKGRFTIS
RDNSKNTLYLQMNSLRAEDTAVYYCAKGVHYDSSGLPIDWYFDLWGRGTLVTVSS
>M0072-E03-R0032-C03-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA
>M0072-G06-R0032-C05-LV
QDIQMTQSPSTLSASVGDRVTITCRASQTISSWLAWYQQKPGKAPKLMIYKAASLGSEVPSRFSGSGSGTE
FTLTISSLQPEDFSTYYCQQYKTYPVTFGQGTRLEIK
>M0072-G06-R0032-C05-LC
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC..
>M0072-G06-R0032-C05-HV
EVQLLESGGGLVQPGGSLRLSCAASGFTFSEYVMWVRQAPGKGLEWVSVISPSGGYTVYADSVKGRFTIS
RDNSKNTLYLQMNSLRAEDTAVYYCARDRGGATTLDYWGQGTLVTVSS
>M0072-G06-R0032-C05-HC
ASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCAAAHHHHHHGAAEQKLISEEDLNGAAEASSASNA

### Example 4

### Germlining and Production of Anti-IGF-II/IGF-IIE IgGs

2 IgG's were germlined. Both were derived from VK1_{—}02 DPK9/02. M0064-E04 required 4 changes overall 3 in the light chain proper and 1 in JK5 and M0064-F02 required 3 changes in the light chain proper and none in JK1.

The amino acid changes made in the germlining are illustrated below. The changes were made in the framework regions.

### M0064-E04):

### >VK1_O2 DPK9/O2

| Length = 95; Score = 174 bits (442), Expect = 7e−48 | | | |
|---|---|---|---|
| Identities = 86/95 (90%), Positives = 89/95 (93%) | | | |
| Query: | 2 | DIQMTQSPSSLSASVGDRVTITC QASHDISNYLN WYQQKPGKAPKLLIY AASRLQSGVPS | 61 |
| | | DIQMTQSPSSLSASVGDRVTITC +AS IS+YLN WYQQKPGKAPKLLIY AAS LQSGVPS | |
| Sbjct: | 1 | DIQMTQSPSSLSASVGDRVTITC RASQSISSYLN WYQQKPGKAPKLLIY AASSLQSGVPS | 60 |
| Query: | 62 | RFSGGGSGTDFSLTISSLQAEDFATYYC QQSYSFP | 96 |
| | | RFSG GSGTDF+LTISSLQ EDFATYYC QQSYS P | |
| Sbjct: | 61 | RFSGSGSGTDFTLTISSLQPEDFATYYC QQSYSTP | 95 |

### >JK5

Length = 12; Score = 24.3 bits (51),
Expect = 4e−05
Identities = 10/11 (90%), Positives = 10/11 (90%)

| | | | |
|---|---|---|---|
| Query: | 98 | T FGQGTNLEIK | 108 |
| | | T FGQGT LEIK | |
| Sbjct: | 2 | T FGQGTRLEIK | 12 |

### M0064-F02-LV

### >VK1_O2 DPK9/O2

| Length = 95; Score = 178 bits (451), Expect = 6e−49 | | | |
|---|---|---|---|
| Identities = 87/95 (91%), Positives = 92/95 (96%) | | | |
| Query: | 2 | DIQMTQSPSSLSASVGDRVTITC RASQSISNYLN WYQQKPGKAPKLLIY TASTLQSGVPS | 61 |
| | | DIQMTQSPSSLSASVGDRVTITC RASQSIS+YLN WYQQKPGKAPKLLIY AS+LQSGVPS | |
| Sbjct: | 1 | DIQMTQSPSSLSASVGDRVTITC RASQSISSYLN WYQQKPGKAPKLLIY AASSLQSGVPS | 60 |
| Query: | 62 | RFSGSASGTDFTLTINSLQPEDFATYSC QQSYNSP | 96 |
| | | RFSGS SGTDFTLTI+SLQPEDFATY C QQSY++P | |
| Sbjct: | 61 | RFSGSGSGTDFTLTISSLQPEDFATYYC QQSYSTP | 95 |

### >JK1

Length = 12; Score = 30.4 bits (67),
Expect = 6e−07
Identities = 12/12 (100%), Positives = 12/12
(100%)

| | | | |
|---|---|---|---|
| Query: | 97 | WT FGQGTKVEIK | 108 |
| | | WT FGQGTKVEIK | |
| Sbjct: | 1 | WT FGQGTKVEIK | 12 |

### Example 5

### Affinity Measurement of Selected Anti-IGF-II/IGF-IIE Fabs and IgGs

TABLE 5
Affinity measurements of Fabs by SPR (Flexchip) (in duplicate)

| Original | | | | |
|---|---|---|---|---|
| isolate | | | | |
| name | IGF-II | IGF-IIE | IGF-II | IGF-IIE |
| M0033-E05 | 4.3E−09 | 8.7E−10 | 5.5E−09 | 4.4E−09 |
| M0063-F02 | 7.7E−10 | 1.2E−10 | 1.6E−09 | 2.0E−10 |
| M0064-E04 | 1.2E−10 | 9.3E−11 | 2.5E−10 | 1.2E−10 |
| M0064-F02 | 4.5E−10 | 3.2E−10 | 1.2E−09 | 2.9E−10 |
| M0068-E03 | 9.3E−10 | 6.6E−10 | — | — |
| M0070-H08 | 3.3E−09 | 3.6E−10 | 1.1E−09 | 3.9E−10 |
| M0072-C06 | 5.2E−09 | 5.2E−10 | 7.0E−10 | — |
| M0072-E03 | 7.5E−10 | 9.6E−11 | 3.0E−10 | 2.2E−10 |
| M0072-G06 | 5.8E−10 | 5.6E−10 | 1.4E−09 | 8.9E−10 |

— = No second affinity measurement obtained

TABLE 6
Affinity measurements of IgGs as measured by SPR (Biacore)

| Non- | | | | | |
|---|---|---|---|---|---|
| germlined | IGF-II | IGF-IIE | Germlined | IGF-II | IGF-IIE |
| M0064- | 0.14 nM | 32 pM | X0008-A01 | 23 pM | <23 pM |
| F02 | | | | | |
| M0064- | 0.52 nM | 0.24 nM | X0005-H01 | 0.52 nM | 0.26 nM |
| E04 | | | | | |

nM = E−09
pM = E−12

### Example 6

### IC50 Determination of Selected Anti-IGF-II/IGF-IIE IgGs

### M0063-F02

The purpose of this study was to test the large production of M0063-F02 IgG for its inhibition on IGF-II or IGF-IIE stimulated BaF3 cell proliferation and IC50 determination.

Cell Culture and Materials:

BaF3 cells were cultured in complete medium (90% RPMI 1640+10% FBS+10 ng/ml IL-3+2 mM L-Alanyl-Glutamine+1×Pen/Strep). Cells at passage 41 were used for proliferation assay. IGF-II (67aa), IGF-IIE (104aa) were at 10 μg/ml in PBS and kept at −70° C. IL-4 was purchased from R&D system, Cat#: 404-ML, and anti-IGF-II antibody was purchased from R&D system, Cat#: MAB292. The anti-IGF-II M0063-F02 was at 6.1 mg/ml. Guava ViaCount Reagent, Cat# 4000-0041, was purchased.

Procedure:
Prepare IGF-II at concentration of 400 ng/ml; IGF-IIE at 800 ng/ml; IgG at 120 μg/ml in PBS.
Pre-incubate 25 μl of IGF-II or IGF-IIE with 25 μl of serially diluted IgGs (final con. from 30 μg/ml down to 0) in a 96-well plate, triplicate for each dose, for 30 min at room temperature in a total volume of 50 μl/well.
Prepare cells:
   Harvest BA/F3 cells by centrifuging at 1100 rpm for 5 min.
   Remove supernatant, and resuspend cells in 20 ml of PBS.
   Count cell density by mixing 10 μl of cell suspension with 10 μl of trypan blue solution and loading 10 μl to a hemocytometer to count cell density and viability.
   Spin down cells from PBS solution at 1100 rpm for 5 min.
   Remove supernatant, and resuspend cells in culture medium without IL-3 at 4×10⁵ cells/ml.
   Add 25 μl of cell suspension to each well of the prepared 96-well plate to make the final cell density at 1×10⁴ cells/well.
   Add 25 μl of IL-4 at 200 ng/ml in IL-3 free medium to each well to make the final con. 50 ng/ml.
   The total volume is 100 μl per well.
   Incubate the plate at 37° C., 5% CO₂ for 48 h.

Guava ViaCount Assay:
Centrifuge the 96-well plate, resuspend cells into 200 μl of Guava ViaCount reagent, mix and incubate for 5 minutes. Transfer into a round-bottom 96-well plate.
Guava ViaCount Analysis.
Calculate the IC50 by using the following equation in Sigmaplot:

f=y0−((a*x)/(IC₅₀+x)).

M0063-F02 IgG demonstrated inhibition on IGF-II and IGF-IIE stimulated BaF3 proliferation in a dose dependent manner. The M0063-F02 IgG inhibited both IGF-II and IGF-IIE stimulated cell proliferation with IC50 values at 2 and 0.85 nM respectively.

### M0064-E04 IgG and M0063-F02 IgG:

The purpose of this study was to test the 2 IgG candidates for inhibition on IGF-II or IGF-IIE stimulated BaF3 cell proliferation and IC50 value comparison.

Cell Culture and Materials:

BaF3 cells were cultured in complete medium (90% RPMI 1640+10% FBS+10 ng/ml IL-3+2 mM L-Alanyl-Glutamine+1×Pen/Strep). Cells at passage 22 were used for proliferation assay.

IGF-II (67aa), IGF-IIE (104aa) were at10 μg/ml in PBS and kept at −70° C. IL-4 was purchased from R&D system, Cat#: 404-ML, and anti-IGF-II antibody was purchased from R&D system, Cat#: MAB292. Anti-IGF-II antibody: R&D system, Cat#: MAB292.

Procedure:
Prepare IGF-II at concentration of 400 ng/ml; IGF-IIE at 800 ng/ml; IgG at 200 μg/ml in PBS.
Pre-incubate of 25 μl of IGF-II or IGF-IIE with 25 μl of 1:2 serial diluted IgGs (from 50 μg/ml down to 0) in a 96-well plate, triplicate for each dose, incubate for 30 min at room temperature in a total volume of 50 μl/well.
Prepare cells:
   Harvest BA/F3 cells by centrifuging at 1100 rpm for 5 min.
   Remove supernatant, and resuspend cells in 20 ml of PBS.
   Count cell density by mixing 10 μl of cell suspension with 10 μl of trypan blue solution and loading 10 μl to a hemocytometer to count cell density and viability.
   Spin down cells from PBS solution at 1100 rpm for 5 min.
   Remove supernatant, and resuspend cells in culture medium without IL-3 at 2×10⁶ cells/ml.
   Add 25 μl of cell suspension to each well of the prepared 96-well plate to make the final cell density at 5×10⁴ cells/well.
   Add 25 μl of IL-4 at 200 ng/ml in IL-3 free medium to each well to make the final con. 50 ng/ml.
   The total volume is 100 μl per well.
   Incubate the plate at 37° C., 5% CO₂ for 48 h.

Guava ViaCount Assay
Mix each well of the 96well plate, take 20 μl of cell sample from each well to a new 96well plate, add 180 μl of Guava ViaCount reagent to each well, mix and incubate for 5 minutes.
Guava ViaCount Analysis.

Both IgGs demonstrated inhibition on IGF-II; IGF-IIE stimulated BaF3 proliferation in a dose dependent manner Neither IgG showed a significant effect on IGF-I stimulated cell proliferation. The M0063-F02 IgG inhibited both IGF-II and IGF-IIE stimulated cell proliferation with IC50 values at −7 and 10 nM respectively, and the M0064-E04 IgG inhibited both IGF-II and IGF-IIE stimulated cell proliferation with IC50 values at −19 and 130 nM respectively.

### Example 7

### In Vitro Studies Using Anti-IGF-II/IGF-IIE Binding Proteins

### IGF-1R Phospho-Assay in MCF-7 Cells

We tested the inhibitory effect of M0063-F02 IgG on IGF-II and/or IGF-IIE induced IGF-1R phosphorylation in MCF-7 cell lines.

MCF-7: breast cancer cell line cells were cultured in MEM media with 10% FBS, 0.1 mM NEAA, 1 mM Na Pyruvate, 0.01 mg/ml bovine Insulin and 1×Pen/Strep. Anti-IGF-1R antibody was purchased from Upstate, Cat#: 05-656 and anti-Phospho-IGF-IR antibody was purchased from Cell Signaling. Cat# 3024. MCF-7 (P15) were cultured in complete medium in 6-well plate at 1×10⁶ cells/well at 37° C., 5% CO2 incubator O/N. The cells were then starved with basal-MEM media for 6 hrs and were treated in batches as follows:
1. No treatment
2. Cells were treated with 10 nM IGF-II for 20 min
3. Cells were treated with 10 nM IGF-IIE for 20 min
4. Cells were pre-treated with 40 nM M0063-F02 IgG for 30 min then IGF-II 10 nM was added for 20 min
5. Cells were pre-treated with 40 nM M0063-F02 IgG for 30 min then IGF-IIE 10 nM was added for 20 min
6. Pre-mixed M0063-F02 40 nM with IGF-II 10 nM for 30 min, then added to cells
7. Pre-mixed M0063-F02 40 nM with IGF-IIE 10 nM for 30 mM, then added to cells
8. F02 40 nM, IGF-II 10 nM were added to cells simultaneously, treated for 20 min
9. M0063-F02 40 nM, IGF-IIE 10 nM were added to cells simultaneously, treated for 20 min
10. Cells were pre-treated with 40 nM A02 IgG for 30 min then IGF-II 10 nM was added for 20 min
11. Cells were pre-treated with 40 nM A02 IgG for 30 min then IGF-IIE 10 nM was added for 20 min
   The cells were washed once with ice-cold PBS containing 1 mM sodium orthvanadate. Cells were lysed with 1 ml of RIPA buffer with protease inhibitor cocktail, and incubated for 10 min on ice. The lysates were spun at 14,000 rpm for 10 min to get rid of the cell debris. Cell lysates were immunoprecipitated with anti-IGF-1R antibody at 2 ug/ml, 20 μl agarose beads at 4° C., 0/N, immunoprecipitates were collected and washed 3 times with 1 ml RIPA buffer. 12 ul of 2× electrophoresis sample buffer was added.

Western blotting: Samples were heated at 70° C., water bath for 10 min, then loaded samples into 15-well 4-12% Bis-Tris gel. Transferred the resolved proteins to a 0.45 um PVDF membrane. The membrane was blocked with 5% BSA-PBST (0.05% Tween 20) at room temperature for 1 hr and robed with anti-phospho-IGF-1R Ab at 1:1000 dilution in 3% BSA-PBS-T o/N 40 C. The membrane was washed 3 times with PBS. Subsequently, the blot was probed with anti-Rabbit-IgG-HRP at 1:5000 dilution in 3% BSA-PBST for 1 hr at room temperature and washed 3 times with PBS. The blot was developed with Supersignal west Femto Maximum Sensitivity Substrate (Pierce 1859022&23) The membrane was stripped and blocked with 5% BSA-PBST (0.05% Tween 20) at room temperature for 1 hr. It was probed with anti-IGF-1R Ab at 1:3000 dilution in 3% BSA-PBS-T 0/N at 40° C. The membrane was washed 3 times with PBS. Subsequently, the blot was probed with anti-mouse IgG-HRP at 1:5000 dilution in 3% BSA-PBST for 1 hr at room temperature and washed 3 times with PBS. The blot was developed with Supersignal west Femto Maximum Sensitivity Substrate (Pierce 1859022&23)

Preliminary results demonstrated that stimulation of MCF-7 cells with IGF-II or IGF-IIE induced IGF-1R phosphorylation under serum free condition. M0063-F02 IgG at 40 nM showed an inhibitory effect on both IGF-II and IGF-IIE induced IGF-1R phosphorylation in MCF-7 cells. The similar inhibitory activity was observed in 3 different protocols: preincubation of the cells with antibody; premixing of the antibody with IGF-II or IGF-IIE and simultaneous addition of IGF-II/IIE and the antibody to cells. A02 as a IgG control showed no effect on IGF-II or IGF-IIE induced IGF-1R phosphorylation.

### M0064-E04 and M0064-F02 IgG Comparison in IGF-1R Phosphorylation Assay

We tested the comparative inhibitory effect of M0064-F02 and M0064-E04 IgG on IGF-II and/or IGF-IIE induced IGF-1R phosphorylation in MCF-7 cell lines.

MCF-7: breast cancer cell line cells were cultured in MEM media with 10% FBS, 0.1 mM NEAA, 1 mM Na Pyruvate, 0.01 mg/ml bovine Insulin and 1×Pen/Strep. Anti-IGF-1R antibody was purchased from Upstate, Cat#: 05-656 and anti-Phospho-IGF-IR antibody was purchased from Cell Signaling. Cat# 3024. MCF-7 (P20) cells were harvested and seeded in complete medium in 6-well plate at 1×10⁶ cells/well at 37° C., 5% CO₂ incubator O/N. The cells were then starved with basal-MEM media for 6 hrs. The cells were treated in batches as follows:
No treatment
Cells were treated for 20 min with 10 nM IGF-II
Cells were treated for 20 min with IGF-II 10 nM plus different dose of M0064-E04, from 40 nM down to 0.16 nM
Cells were treated for 20 min with IGF-II 10 nM plus different dose of M0064-F02, from 40 nM down to 0.16 nM
Cells were treated for 20 min with IGF-II 10 nM plus control IgG A2 40 nM as negative control

The cells were washed once with ice-cold PBS containing 1 mM sodium orthovanadate. Cells were lysed with 1 ml of RIPA buffer with protease inhibitor cocktail, and incubated for 10 min on ice. The lysates were spun at 14,000 rpm for 10 min to get rid of the cell debris. Cell lysates were immunoprecipitated with anti-IGF-1R antibody at 2 ug/ml, 20 μl agarose beads at 4° C., 0/N, immunoprecipitates were collected and washed 3 times with 1 ml RIPA buffer. 12 ul of 2× electrophoresis sample buffer was added.

Western blotting: heated samples at 70° C., water bath for 10 min, then loaded samples into 15-well 4-12% Bis-Tris gel. The resolved proteins were transferred to a 0.45 um PVDF membrane. The membrane was blocked with 5% BSA-PBST (0.05% Tween 20) at room temperature for 1 hr, and probed with anti-phospho-IGF-1R Ab at 1:1000 dilution in 3% BSA-PBS-T O/N 40° C. The membrane was washed 3 times with PBS. Subsequently, the blot was probed with anti-Rabbit-IgG-HRP at 1:5000 dilution in 3% BSA-PBST for 1 hr at room temperature, and washed 3 times with PBS. The blot was developed with Supersignal west Femto Maximum Sensitivity Substrate (Pierce 1859022&23). The membrane was stripped and blocked with 5% BSA-PBST (0.05% Tween 20) at room temperature for 1 hr, and probed with anti-IGF-1R Ab at 1:3000 dilution in 3% BSA-PBS-T O/N 40° C. The membrane was washed 3 times with PBS. Subsequently, the blot was probed with anti-mouse IgG-HRP at 1:5000 dilution in 3% BSA-PBST for 1 hr at room temperature, and washed 3 times with PBS. The blot was developed with Supersignal west Femto Maximum Sensitivity Substrate (Pierce 1859022&23)

Preliminary results demonstrated that stimulation of MCF-7 cells with IGF-II induced IGF-1R phosphorylation under serum free condition. M0064-E04 and M0064-F02 IgGs showed inhibitory effects on IGF-II induced IGF-1R phosphorylation in MCF-7 cells in a dose dependent manner The similar inhibitory potency was observed between those two antibodies. A02 as an IgG negative control showed no effect on IGF-II induced IGF-1R phosphorylation.

### Example 8

### Crystallography and Epitopic Mapping

The crystallographic structure of IGF-II with M0064-F02 was determined in order to characterize the epitopic region of the IGF2 to which the antibody binds. Crystals were obtained using 1-10 mg/mL IGF-II with the M0064-F02 Fab in a molar ratio of 2:1 in mid-weight PEG conditions, pH ˜5 with either Ca++ or L1++ as additives.

Crystallization statistics were as follows:

Cell: 50.22 106.67 110.89 90.00 90.00 90.00

Space Group: P2₁2₁2₁

Number of Atoms: 4050 (233 water molecules)

% Solvent: 52.67

<B> for atomic model: 33.85

Sigma(B): 9.21

Resolution: 49.21-2.40

Reported R factor: 0.185

Rfree: 0.261

Number max possible refls: 24010 Actual: 22775

Completeness: 94.9%

Correlation factor: 0.9254

The Fab structure was solved using molecular replacement with pdb #1igf and the IGF-II structure solved using pdb 2v5p.

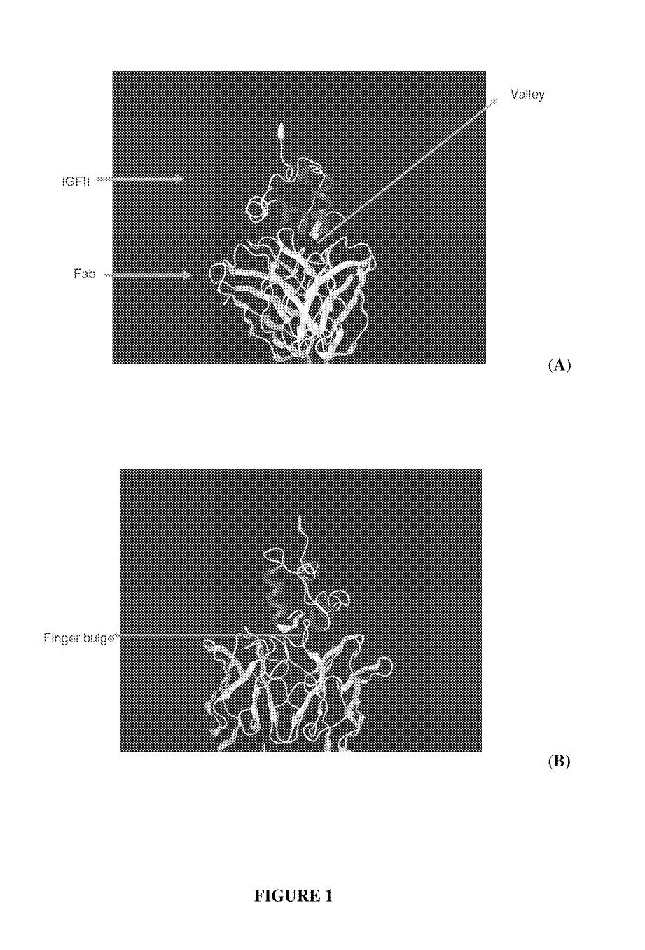

Views of the structure are depicted in FIG. 1. One valley appears to be important in the Fab surface for binding to IGF-II. One encloses the residues Cys9 through Gly11 and buries the Cys9-Cys47 disulfide bridge and with a bump also residue Phe48. The second valley is on the other side of the Tyr103H bulge and the residues here line the top of the valley but are not found deep within. This valley has a negative electrostatic potential, but there are no positively charged residues that delve into this area to offset this charge. There are two Arg residues (37 and 38) with their side chains pointing into space that do not make hydrogen (H)-bonds nor ionic interactions with Asp102H and the further buried Glu106H and Asp99H residues. The closest contacts seem to be the N epsilon of Arg34 to the Val35 backbone nitrogen at 4.4 A and the carboxylic acid group of Asp102 at 4.7 A.

The Met57H residue seems to be covered on three sides by mostly charged residues (Glu44, Glu45, Arg49), but the charges are all pointing away from the Met and only the aliphatic carbons seem to contribute to the binding surface, which also includes the uncharged Phe48.

The most prominent feature on the Fab is the finger bulge made by Tyr103H. Although this sticks a bit like a finger into IGF-II, there is still a gap or hole left between the two molecular surfaces that might be filled to a certain extent by a larger residue such as Trp. It is a hydrophobic pocket on the IGF-II surface made up of Tyr59, Phe26, Leu17, Leu13, Val43, Val14.

Table 7 below shows the partial sequence of IGF-II, the bolded amino acids being those that have been shown through crystallographic studies to be involved with the binding of the Fab:

TABLE 7
>P01344|IGF2_HUMAN Insulin-like growth factor II
partial seq
5
SETLCGGELVDTLQFVCGDRGFYFSRPASRVSRRSRGIVEECCFRSCD
64
LALLETYCATPA.

Reading from this partial sequence it can be seen that residues from IGF-II contribute to the binding surface and are designated as T7, C9, G10, G11, L13, V14, L17, F26, P31, R34, V35, R37, S39, R40, G41, V43, E44, E45, C47, F48, R49, Y59

Hydrogen bonds (with angstrom distances being between 2.60 Å and 3.84 Å) found between the heavy chains (H) and IGF-II (D) are as follows:

H: S53-D: E44

H: R59-D: C9

H: R59-D: C47

H: R59-D: T7

H: Y103-D: Y59

H: N31-D: R40

H: G56-D: R49

H: Y103-D: G10

Residues contributing to the binding surface from the heavy chain are as follows:

S30, N31, I33, V50, I51, S52, S53, S54, G56, M57, T58, R59, D102, Y103, V104, G105, E106

Hydrogen bonds (distances 2.88 Å to 3.52 Å) found between the light chain (L) and IGF-II (D) are as follows:

L: Y33-D: D15

L: S92-D: G11

L: Y93-D: G11

L: Y93-D: E12

Residues contributing to the binding surface from the light chain:

S31, N32, Y33, S92, Y93, N94, S95, W97

### Example 9

### Affinity in Solution Measurements for Fabs Binding to IGF-II and IGF-IIE

Competition SPR (BIACORE®) analysis was utilised
1) to demonstrate that Fab fragments isolated from phage display library specifically inhibited binding of IGF-II and IGF-IIE ligands to immobilized full-length human insulin receptor-A ectodomain (huIR-A ECD) and
2) to estimate in solution affinities of these Fabs.

### Method

Human IR-A ECD comprising residues 1-914 of the mature protein together with a C-terminal myc epitope tag, was isolated and purified from bioreactor cultures of stably transfected Lec 8 cells (a glycosylation mutant derived from CHO-K1 cells) as described methodology (J Struct Biol. 1999 March; 125(1):11-8.)

Competition SPR (BIACORE®) was performed under conditions of partially mass-transport limited conditions according to previously described methodology (Nieba et al., 1996). Approximately 16,700 relative response units (RU) of huIR-A (-exon11) ectodomain was coupled by standard amine chemistry to a BIACORE® CM5 chip sensor. Uncoated flow-cell surface was used as a reference. Each binding/regeneration cycle between IGF-II ligands and immobilized huIR-A ECD was performed at 25° C. with a constant flow rate of 30 ul/min in HBS-EP+running buffer (10 mM HEPES, pH 7.4, 150 mM NaCl, 3.4 mM EDTA, 0.05% Tween-20). Regeneration of the surface was achieved by injection of 30 ul NaCltrate/NaCl pH 4.5. Initially, 60 ul samples containing increasing amounts of IGF-II (or IGF-IIE) ligand (typically increasing two-fold from 0.5 to 8 nM) in HBS-EP+buffer were injected over the immobilized huIR-A ECD and their overall binding responses (5 sec after injection stopped) used to establish standard binding curve. To demonstrate inhibition of IGF-II binding by Fab fragments and to derive affinity in solution values (K_{D}), the IGF-II (or IGF-IIE) ligand was pre-incubated with Fab fragments at different concentrations in a constant final volume of 120 ul for at least 1 h at 25° C. before injection. 60 ul samples of these equilibrated mixtures were injected over immobilized huIR-A ECD and the overall binding response generated by binding of free IGF-II ligands to huIR-A ECD recorded 5 seconds after injection stopped. Binding data were evaluated using the Biacore T100 Evaluation software (GE Healthcare) whereby these overall binding responses together with standard curve described above were used to derive concentrations of free IGF-II ligand in solution at equilibrium (Req). These Req estimates were subsequently plotted against total concentrations of Fab used and the resulting inhibition curve was utilised to calculate dissociation constant (K_{D}).

Results:

TABLE 8
Affinity in solution (KD's) estimates of Fabs (BIACORE ® T100).

| Original | Measurement#1 (KD nM) | | Measurement#2 (KD nM) | |
|---|---|---|---|---|
| Isolate name | IGF-II | IGF-IIE | IGF-II | IGF-IIE |
| M0033-E05 | 4.32E−09 | 8.21E−09 | — | — |
| M0063-F02 | 1.17E−09 | 2.78E−09 | 8.58E−10 | 1.47E−09 |
| M0064-E04 | 6.08E−10 | 4.29E−10 | 1.11E−09 | 4.24E−10 |
| M0064-F02 | 1.03E−09 | 1.91E−09 | 5.03E−10 | 3.52E−10 |
| M0068-E03 | 5.76E−09 | 5.55E−09 | — | — |
| M0070-H08 | 3.24E−09 | 4.83E−09 | — | — |
| M0072-C06 | 1.64E−09 | 3.95E−09 | 2.52E−09 | 1.05E−09 |
| M0072-E03 | 2.62E−09 | 5.32E−09 | — | — |
| M0072-G06 | 3.99E−09 | 6.36E−09 | — | — |
| M0080-G03 | No Inhibition | 2.93E−08 | No Inhibition | 2.12E−08 |
| M0073-C11 | No Inhibition | 2.02E−08 | No Inhibition | 1.91E−08 |

Results demonstrated that most of these Fabs inhibited binding of IGF-II and IGF-IIE to the huIR-A ECD receptor. Two Fabs shown here (M0080-G03 and M0073-C11) inhibited binding of IGF-IIE only whilst no inhibition of IGF-II binding was observed, thus demonstrating that binding of these two Fabs was specific to IGF-IIE.

### Example 10

### Anti-IGFII (and IGF-IIE) Antibody Competition Assay Against Binding Proteins (BP2 and BP4)

IGF binding proteins (BPs) play a major role in modulating the actions of IGF-I and IGF-II (and IGF-IIE) on cells. They can either enhance or inhibit the action of IGF on cells. IGF BP2 preferentially binds IGF-II over IGF-I and is secreted by a variety of cells. Similarly, IGF BP4 acts as a scavenger of IGFs and is an inhibitor of IGF action.

In this example, the candidate IgG's interaction with BP2 and BP4 and their competitive binding to IGF-II and IGF-IIE was investigated.

Method:

BIACORE® T100 and a CM5 chip coated with Protein G were utilised to set up following assay. Approximately 1000 RU of Protein G′ was immobilized on a CM5 chip using standard amine chemistry immobilization method.
Entire assay consisted of three sequential injection of following four reagents
1) Candidate IgG was injected onto Protein G′ at 10 ug/ml at 5 ul/min for 180 sec. This typically resulted in a capture of ˜3000 RU of IgG.
2) IGF-II ligand (at 50 nM) was then injected at 5 ul/min for 90 sec. This allowed for binding of IGF-II to the captured antibody.
3) BP2 or BP4 (also at 50 nM) were then injected at 30 ul/min for 90 sec. Significant binding response upon injection of BP was indicative of candidate antibody and BP binding to IGF-II in non-competitive manner
4) Protein G′ surface was finally regenerated by injection of 10 mM Glycine pH1.5-injected at 30ul/min for 60 sec.

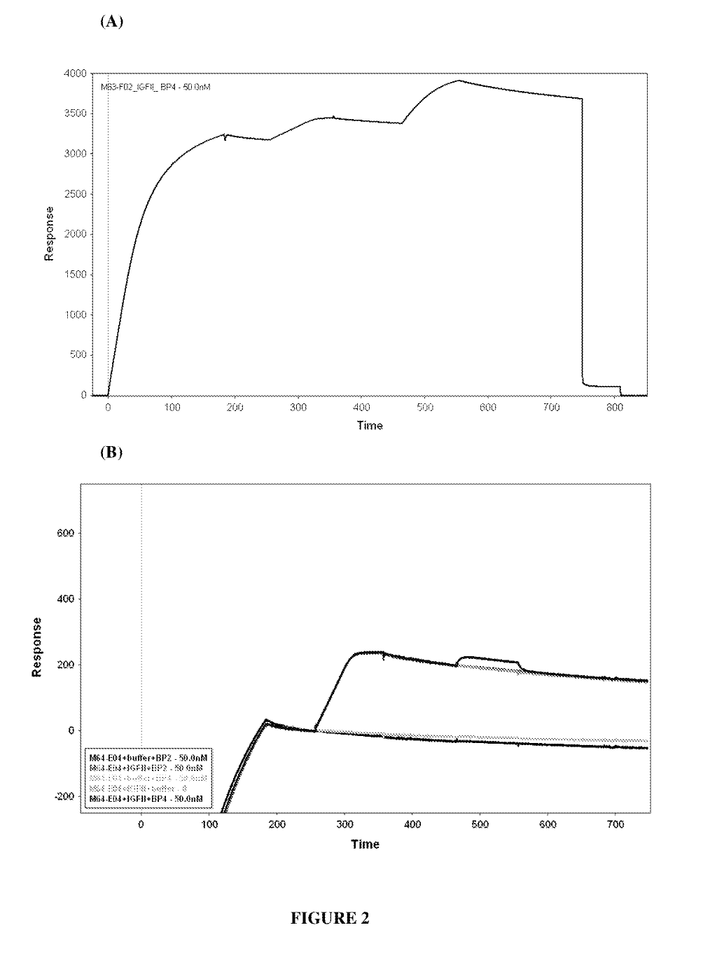

FIG. 2A gives a typical binding profile obtained for one of the candidate antibodies. M0063-F02 appeared to bind to IGFII in a non-competitive manner with respect to BP4.

FIG. 2B shows competition binding data for M0064-E04 candidate antibody. Following controls were included to ascertain these binding results (refer to legend—inset bottom left)
1) no IGF-II injected (instead inject BIACORE® running buffer only) Confirmed that BP2/BP4 do not bind captured antibody
2) no BP2 or BP4 was injected after capturing IGF-II Established the baseline for IGF-II dissociation from antibody
3) control antibody that does not bind IGF-II (w02 murine antibody) to confirm IGF-II and/or BP2/BP4 do not significantly interact with either Protein G′ or chip surface.

The following table shows the summary for these competition binding results for candidate antibodies against IGF-II ligand with respect to BP2, BP4.

TABLE 9
Scoring system scales:
5 = candidate IgG competes strongly with BP for binding to IGF-II ligand
0 = candidate IgG does not competes significantly with BP for
binding to IGF-II ligand

| | |
|---|---|
| IGF-II | IGF-IIE |

| Candidate Antibody | BP2 | BP4 | BP2 | BP4 |
|---|---|---|---|---|
| M0063-F02 | 1 | 0 | 1 | 1 |
| M0064-E04 | 5 | 5 | 4 | 4 |
| M0064-F02 | 4 | 4 | 4 | 4 |
| M0070-H08 | 3 | 2 | 2 | 1 |
| M0072-C06 | 3 | 2 | 2 | 1 |
| M0072-E03 | 4 | 4 | 3 | 3 |
| M0072-G06 | 2 | 1 | 2 | 1 |

| | | | |
|---|---|---|---|
| M0080-G03 | does not bind to IGF-II | 5 | 5 |
| 566A-M0073-C11 | does not bind to IGF-II | 4 | 4 |

In Summary:

IGFII Ligand Competition:

M0064-E04 IgG appears to be best competing antibody with BP2 and BP4 for binding to IGFII ligand. M0064-F02 and M0072-E03 are the next two best antibodies.

### Example 11

### DNA and Amino Acid Sequences of Anti-IGF-IIE Fabs

M0073-C11

>566A-X0003-B02 (566A-M0073-C11) LV+LC

CAGAGCGCTTTGACTCAGCCACCCTCAGTGTCCGTGTCCCCAGGAC
AGACAGCCAGCATCACCTGCTCTGGAGATAGATTGGGGGATAAATATGCT
TCCTGGTATCAGCAGAAGCCAGGCCAGTCCCCTGTGTTGGTCATCTATCA
AGATACCAAGCGGCCCTCAGGGATCCCTGAGCGATTCTCTGGCTCCAACT
CTGGGTCCACAGCCACTCTGACCATCAGCGGGACCCAGGCTATGGATGAG
GCTGACTATTACTGTCAGGCGTGGGACAGCAGCACTGTGGTATTCGGCGG
AGGGACCAAGCTGACCGTCCTAGGTCAGCCCAAGGCTGCCCCC

>566A-X0003-B02 (566A-M0073-C11) HV+HC

GAAGTTCAATTGTTAGAGTCTGGTGGCGGTCTTGTTCAGCCTGGT
GGTTCTTTACGTCTTTCTTGCGCTGCTTCCGGATTCACTTTCTCTCCTTA
CGATATGTGGTGGGTTCGCCAAGCTCCTGGTAAAGGTTTGGAGTGGGTTT
CTTCTATCTCTTCTTCTGGTGGCATTACTGCTTATGCTGACTCCGTTAAA
GGTCGCTTCACTATCTCTAGAGACAACTCTAAGAATACTCTCTACTTGCA
GATGAACAGCTTAAGGGCTGAGGACACGGCCGTGTATTACTGTGCGAGGG
CCGGGTATAGCAGCAGCTGGGGGTACTACTACTACGGTATGGACGTCTGG
GGCCAAGGGACCACGGTCACCGTCTCAAGCGCCTCCACCAAGGGCCCATC
GGTCTTCCCGCTAGCACCCTCCTCCAAGAGC

>566A-X0003-B02 (566A-M0073-C11) LV+LC

QSALTQPPSVSVSPGQTASITCSGDRLGDKYASWYQQKPGQSPVL
VIYQDTKRPSGIPERFSGSNSGSTATLTISGTQAMDEADYYCQAWDSSTV
VFGGGTKLTVLGQPKAAP

>566A-X0003-B02 (566A-M0073-C11) HV+HC

EVQLLESGGGLVQPGGSLRLSCAASGFTFSPYDMWWVRQAPGKGL
EWVSSISSSGGITAYADSVKGRFTISRDNSKNTLYLQMNSLRAEDTAVYY
CARAGYSSSWGYYYYGMDVWGQGTTVTVSSASTKGPSVFPLAPSSKS

M0080-G03

>566A-X0004-A02 (566A-M0080-G03) LV+LC

CAGAGCGTCTTGACTCAGCCACCCTCAGCGTCTGGGACCCCCGGG
CAGAGGGTCACCATCTCTTGTTCTGGAAGCAGCTCCAACATCGGAAGTTA
TTATGTATATTGGTACCAGCAGATCCCAGGAACGGCCCCCAAACTCCTCA
TCTATAGGAATAATCAGCGGCCCTCAGGGGTCCCTGACCGATTCTCTGGC
GCCAAGTCTGGCACCTCAGCCTCCCTGGCCATCAGTGGGCTCCGGTCCGA
GGATGAGGCTGATTATTACTGTGCAGCATGGGATGACAGCCTCCATGGTT
GGGTTTTCGGCGGAGGGACCAAGCTGACCGTCCTAGGTCAGCCCAAGGCT
GCCCCC

>566A-X0004-A02 (566A-M0080-G03) HV+HC

GAAGTTCAATTGTTAGAGTCTGGTGGCGGTCTTGTTCAGCCTGGT
GGTTCTTTACGTCTTTCTTGCGCTGCTTCCGGATTCACTTTCTCTTTTTA
CCAGATGATGTGGGTTCGCCAAGCTCCTGGTAAAGGTTTGGAGTGGGTTT
CTTCTATCTCTCCTTCTGGTGGCCGTACTTATTATGCTGACTCCGTTAAA
GGTCGCTTCACTATCTCTAGAGACAACTCTAAGAATACTCTCTACTTGCA
GATGAACAGCTTAAGGGCTGAGGACACGGCCGTGTATTACTGTGCAAAAG
ATATTGGGCTAGGATATTGTAGTAGTACCAGCTGCTATACGGGTACCCCT
CTTGACTACTGGGGCCAGGGCACCCTGGTCACCGTCTCAAGCGCCTCCAC
CAAGGGCCCATCGGTCTTCCCGCTAGCACCCTCCTCCAAGAGC

>566A-X0004-A02 (566A-M0080-G03) LV+LC

QSVLTQPPSASGTPGQRVTISCSGSSSNIGSYYVYWYQQIPGTAP
KLLIYRNNQRPSGVPDRFSGAKSGTSASLAISGLRSEDEADYYCAAWDDS
LHGWVFGGGTKLTVLGQPKAAP

>566A-X0004-A02 (566A-M0080-G03) HV+HC

EVQLLESGGGLVQPGGSLRLSCAASGFTFSFYQMMWVRQAPGKGL
EWVSSISPSGGRTYYADSVKGRFTISRDNSKNTLYLQMNSLRAEDTAVYY
CAKDIGLGYCSSTSCYTGTPLDYWGQGTLVTVSSASTKGPSVFPLAPSSK
S

### Example 12

### Exemplary IGF II/IGF IIE Inhibitory Binding Proteins

DX-2647

DX-2647 is an exemplary IGF II/IGF IIE inhibitory antibody. DX-2647 is germlined from 566A-M0064-F02 parental clone. The DNA and amino acid sequences of DX-2647 are as follows.

>DX-2647 LV+LC

DIQMTQSPSSLSASVGDRVTITCRASQSISNYLNWYQQKPGKAPKLLIYT
ASTLQSGVPSRFSGSGSGTDFTLTISSLQPEDFATYYCQQSYNSPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

>DX-2647 HV+HC

EVQLLESGGGLVQPGGSLRLSCAASGFTFSNYIMWWVRQAPGKGLEWVSV
ISSSGGGTLYADSVKGRFTISRDNSKNTLYLQMNSLRAEDTAVYYCARDN
GDYVGEKGFDIWGQGTMVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

>DX-2647 LV+LC

GACATCCAGATGACCCAGTCCCCCAGCTCCCTGTCTGCTTCCGTGGGCGA
CCGGGTGACCATCACCTGCCGGGCCTCCCAGTCCATCTCCAACTACCTGA
ACTGGTATCAGCAGAAGCCTGGCAAGGCCCCTAAGCTGCTGATCTACACC
GCCTCTACACTGCAGTCTGGAGTCCCTTCCAGGTTCTCCGGCTCCGGCAG
CGGCACCGACTTCACCCTGACCATCTCCTCCCTGCAGCCTGAGGACTTCG
CCACCTACTACTGCCAGCAGTCCTACAACTCCCCTTGGACCTTCGGCCAG
GGCACCAAGGTGGAGATCAAGCGGACCGTGGCCGCTCCTTCCGTGTTCAT
CTTCCCTCCCTCCGACGAGCAGCTGAAATCCGGCACTGCCAGCGTGGTCT
GCCTGCTGAACAACTTCTACCCTCGGGAGGCCAAGGTGCAGTGGAAGGTG
GACAACGCCCTGCAGAGCGGCAACTCCCAGGAATCCGTCACCGAGCAGGA
CTCCAAGGACAGCACCTACTCCCTGTCCAGCACCCTGACCCTGTCCAAGG
CCGACTACGAGAAGCACAAGGTGTACGCCTGCGAGGTGACCCACCAGGGC
CTGTCCTCCCCCGTGACCAAGTCCTTCAACCGGGGCGAGTGC

>DX-2647 HV+HC

GAGGTGCAATTGCTGGAGTCTGGCGGCGGACTGGTGCAGCCTGGCGGCTC
CCTGCGGCTGTCCTGCGCCGCCTCCGGCTTCACCTTCTCCAACTACATCA
TGTGGTGGGTGCGGCAGGCTCCTGGAAAGGGCCTCGAGTGGGTGTCCGTG
ATCTCCAGCTCCGGGGGAGGAACACTGTACGCCGACTCCGTGAAGGGCCG
GTTCACCATCTCCAGAGACAACTCCAAGAACACCCTGTACCTGCAGATGA
ACTCCCTGCGGGCCGAGGACACCGCCGTGTACTACTGCGCCAGGGACAAC
GGCGACTACGTGGGCGAGAAGGGCTTCGACATCTGGGGCCAGGGCACAAT
GGTGACCGTGTCCTCCGCCTCCACCAAGGGCCCTTCCGTGTTCCCGCTAG
CACCTTCCTCCAAGTCCACCTCTGGCGGCACCGCCGCTCTGGGCTGCCTG
GTGAAGGACTACTTCCCTGAGCCTGTGACCGTGAGCTGGAACTCTGGCGC
CCTGACCTCCGGCGTGCATACCTTCCCTGCCGTGCTGCAGTCCTCCGGCC
TGTACTCCCTGTCCTCCGTGGTGACAGTGCCTTCCTCCTCCCTGGGCACC
CAGACCTACATCTGCAACGTGAACCACAAGCCTTCCAACACCAAGGTGGA
CAAGCGGGTGGAGCCTAAGTCCTGCGACAAGACCCACACCTGCCCTCCCT
GCCCTGCCCCTGAGCTGCTGGGCGGACCCTCCGTGTTCCTGTTCCCTCCT
AAGCCTAAGGACACCCTGATGATCTCCCGGACCCCTGAGGTGACCTGCGT
GGTGGTGGACGTGTCCCACGAGGACCCAGAGGTGAAGTTTAATTGGTATG
TGGACGGCGTGGAGGTCCACAACGCCAAGACCAAGCCTCGGGAGGAACAG
TACAACTCCACCTACCGGGTGGTGTCCGTGCTGACCGTGCTGCACCAGGA
CTGGCTGAACGGCAAGGAATACAAGTGCAAAGTCTCCAACAAGGCCCTGC
CTGCCCCCATCGAGAAAACCATCTCCAAGGCCAAGGGCCAGCCTCGCGAG
CCTCAGGTGTACACCCTGCCTCCTAGCCGGGAGGAAATGACCAAGAACCA
GGTGTCCCTGACCTGTCTGGTGAAGGGCTTCTACCCTTCCGATATCGCCG
TGGAGTGGGAGTCCAACGGCCAGCCTGAGAACAACTACAAGACCACCCCT
CCTGTGCTGGACTCCGACGGCTCCTTCTTCCTGTACTCCAAGCTGACCGT
GGACAAGTCCCGGTGGCAGCAGGGCAACGTGTTCTCCTGCTCCGTGATGC
ACGAGGCCCTGCACAACCACTACACCCAGAAGTCCCTGTCCCTGAGCCCT
GGCAAG

The light and heavy chain framework, CDR and constant region sequences are as follows. A protein containing the 6 CDRs from DX-2647 can be used in the compositions and methods described herein.

| | | | LV- | |
|---|---|---|---|---|
| LV-FR1 | LV-CDR1 | LV-FR2 | CDR2 | LV-FR3 |
| DIQMTQSPSSLSASVGDRVTITC | RASQSISNYLN | WYQQKPGKAPKLLIY | TASTLQS | GVPSRFSGSGSGTDFTLTISSLQPEDFATYYC |

| LV-CDR3 | LV-FR4 | L-Constant |
|---|---|---|
| QQSYNSPWT | FGQGTKVEIK | RTVAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |

| | HV- | | | |
|---|---|---|---|---|
| HV-FR1 | CDR1 | HV-FR2 | HV-CDR2 | HV-FR3 |
| EVQLLESGGGLVQPGGSLRLSCAASGFTFS | NYIMW | WVRQAPGKGLEWVS | VISSSGGGTLYADSVKG | RFTISRDNSKNTLYLQMNSLRAEDTAVYYCAR |

| HV-CDR3 | HV-FR4 | H-Constant |
|---|---|---|
| DNGDYVGEKGFDI | WGQGTMVTVSS | ASTKGPSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKRVEPKSCDKT |
| | | HTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKSLSLSPGK |

LV-AA

DIQMTQSPSSLSASVGDRVTITCRASQSISNYLNWYQQKPGKAPKLLIYT
ASTLQSGVPSRFSGSGSGTDFTLTISSLQPEDFATYYCQQSYNSPWTFGQ
GTKVEIK

HV-AA

EVQLLESGGGLVQPGGSLRLSCAASGFTFSNYIMWWVRQAPGKGLEWVSV
ISSSGGGTLYADSVKGRFTISRDNSKNTLYLQMNSLRAEDTAVYYCARDN
GDYVGEKGFDIWGQGTMVTVSS

Differences between the germlined and non-germlined LV-FR3 region are shown below.

| | LV-FR3 |
|---|---|
| germlined | GVPSRFSGSGSGTDFTLTISSLQPEDFATYYC |
| Non- | GVPSRFSGSASGTDFTLTINSLQPEDFATYSC |
| germined | |

Bold are non-germlined amino acids found in parental 566A-M0064-F02 Fab.

DX-2655

DX-2655 is an exemplary IGF II/IGF IIE inhibitory antibody. DX-2655 is germlined from 566A-M0064-E04 parental clone. The DNA and amino acid sequences of DX-2655 are as follows.

>566A-X0009-D01 (DX-2655) LV+LC

DIQMTQSPSSLSASVGDRVTITCQASHDISNYLNWYQQKPGKAPKLLIYA
ASRLQSGVPSRFSGSGSGTDFTLTISSLQPEDFATYYCQQSYSFPRTFGQ
GTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

>566A-X0009-D01 (DX-2655) HV+HC

EVQLLESGGGLVQPGGSLRLSCAASGFTFSVYDMNWVRQAPGKGLEWVSS
ISSSGGGTLYADSVKGRFTISRDNSKNTLYLQMNSLRAEDTAVYYCARDS
DTSSYYWYYDLWGRGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

>566A-X0009-D01 (DX-2655) LV+LC

GATATCCAGATGACCCAGTCCCCCAGCTCCCTGTCCGCTAGCGTGGGCGA
CCGGGTGACCATCACCTGCCAGGCCTCCCACGACATCTCCAACTACCTGA
ACTGGTATCAGCAGAAGCCTGGCAAGGCCCCTAAGCTGCTGATCTACGCC
GCCAGCAGACTGCAGTCCGGCGTCCCTAGCCGGTTCTCCGGCTCCGGCAG
CGGCACCGACTTCACCCTGACCATCTCCTCCCTGCAGCCTGAGGACTTCG
CCACCTACTACTGCCAGCAGTCCTACTCCTTCCCTCGGACCTTCGGCCAG
GGCACCCGGCTGGAGATCAAGCGGACCGTGGCCGCTCCTTCCGTGTTCAT
CTTCCCTCCCTCCGACGAGCAGCTGAAGAGCGGCACAGCCAGCGTCGTGT
GCCTGCTGAACAACTTCTACCCTCGGGAGGCCAAGGTGCAGTGGAAGGTG
GACAACGCCCTGCAGAGCGGCAACTCCCAGGAATCCGTCACCGAGCAGGA
CTCCAAGGACAGCACCTACTCCCTGTCCTCCACCCTGACCCTGT

### CCAAGGCCGACTACGAGAAGCACAAGGTGTACGCCTGCGAGGTGACCCACCAGGGCCTGTCCAGCCCT GTGACCAAGTCCTTCAACCGGGGCGAGTGC

>566A-X0009-D01 (DX-2655) HV+HC

GAGGTGCAATTGCTGGAGTCTGGCGGCGGACTGGTGCAGCCTGGCGGCTC
CCTGCGGCTGTCCTGCGCCGCCTCCGGCTTCACCTTCTCCGTGTACGACA
TGAACTGGGTGCGGCAGGCTCCTGGAAAGGGCCTCGAGTGGGTGTCCTCC
ATCTCCAGCTCCGGGGGAGGAACACTGTACGCCGACTCCGTGAAGGGCCG
GTTCACCATCTCCAGAGACAACTCCAAGAACACCCTGTACCTGCAGATGA
ACTCCCTGCGGGCCGAGGACACCGCCGTGTACTACTGCGCCAGGGACTCC
GACACCTCCTCCTACTACTGGTACTACGACCTGTGGGGCAGGGGCACCCT
GGTGACCGTGTCCTCCGCCTCCACCAAGGGCCCTTCCGTGTTCCCGCTAG
CACCTTCCTCCAAGTCCACCTCTGGCGGCACCGCCGCTCTGGGCTGCCTG
GTGAAGGACTACTTCCCTGAGCCTGTGACCGTGAGCTGGAACTCTGGCGC
CCTGACCTCCGGCGTGCATACCTTCCCTGCCGTGCTGCAGTCCTCCGGCC
TGTACTCCCTGTCCTCCGTGGTGACAGTGCCTTCCTCCTCCCTGGGCACC
CAGACCTACATCTGCAACGTGAACCACAAGCCTTCCAACACCAAGGTGGA
CAAGCGGGTGGAGCCTAAGTCCTGCGACAAGACCCACACCTGCCCTCCCT
GCCCTGCCCCTGAGCTGCTGGGCGGACCCTCCGTGTTCCTGTTCCCTCCT
AAGCCTAAGGACACCCTGATGATCTCCCGGACCCCTGAGGTGACCTGCGT
GGTGGTGGACGTGTCCCACGAGGACCCAGAGGTGAAGTTTAATTGGTATG
TGGACGGCGTGGAGGTCCACAACGCCAAGACCAAGCCTCGGGAGGAACAG
TACAACTCCACCTACCGGGTGGTGTCCGTGCTGACCGTGCTGCACCAGGA
CTGGCTGAACGGCAAGGAATACAAGTGCAAAGTCTCCAACAAGGCCCTGC
CTGCCCCCATCGAGAAAACCATCTCCAAGGCCAAGGGCCAGCCTCGCGAG
CCTCAGGTGTACACCCTGCCTCCTAGCCGGGAGGAAATGACCAAGAACCA
GGTGTCCCTGACCTGTCTGGTGAAGGGCTTCTACCCTTCCGATATCGCCG
TGGAGTGGGAGTCCAACGGCCAGCCTGAGAACAACTACAAGACCACCCCT
CCTGTGCTGGACTCCGACGGCTCCTTCTTCCTGTACTCCAAGCTGACCGT
GGACAAGTCCCGGTGGCAGCAGGGCAACGTGTTCTCCTGCTCCGTGATGC
ACGAGGCCCTGCACAACCACTACACCCAGAAGTCCCTGTCCCTGAGCCCT
GGCAAG

| LV-CDR1 | LV-FR2 | LV-CDR2 | LV-FR3 |
|---|---|---|---|
| QASHDISNYLN | WYQQKPGKAPKLLIY | AASRLQS | GVPSRFSGSGSGTDFTLTISSLQPEDFATYYC |

| LV-CDR3 | LV-FR4 | L-Constant |
|---|---|---|
| QQSYSFPRT | FGQGTRLEIK | RTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |

| | HV- | | | |
|---|---|---|---|---|
| HV-FR1 | CDR1 | HV-FR2 | HV-CDR2 | HV-FR3 |
| EVQLLESGGGLVQPGGSLRLSCAASGFTFS | VYDMN | WVRQAPGKGLEWVS | SISSSGGGTLYADSVKG | RFTISRDNSKNTLYLQMNSLRAEDTAVYYCAR |

| HV-CDR3 | HV-FR4 | H-Constant |
|---|---|---|
| DSDTSSYYWYYDL | WGRGTLVTVSS | ASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKRVE |
| | | PKSCDKTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEALHNHYTQKSLSLSPGK |

Differences between the germlined and non-germlined LV-FR3 region are shown below.

| | LV-FR3 |
|---|---|
| germlined | GVPSRFSGSGSGTDFTLTISSLQPEDFATYYC |
| Non- | GVPSRFSGGGSGTDFSLTISSLQAEDFATYYC |
| germined | |

Bold are non-germlined amino acids found in parental 566A-M0064-E04 Fab.

Differences between the germlined and non-germlined LV-FR4 region are shown below.

| | LV-FR4 |
|---|---|
| germlined | FGQGTRLEIK |
| Non- | FGQGTNLEIK |
| germined | |

Bold = non germlined aa found in M064-E04

### REFERENCES

The contents of all cited references including literature references, issued patents, published or non-published patent applications cited throughout this application as well as those listed below are hereby expressly incorporated by reference in their entireties. In case of conflict, the present application, including any definitions herein, will control.

### EQUIVALENTS

A number of embodiments of the invention have been described. Nevertheless, it will be understood that various modifications may be made without departing from the spirit and scope of the invention. Accordingly, other embodiments are within the scope of the following claims.

1. A method of treating or preventing systemic sclerosis-associated pulmonary fibrosis in a subject, the method comprising:
administering an isolated antibody that binds IGF II and/or IGF IIE to the subject, wherein the antibody binds the same epitope or competes for binding with an antibody selected from the group consisting of DX-2647, M0033-E05, M0063-F02, M0064-E04, M0064-F02, M0068-E03, M0070-H08, M0072-006, M0072-E03, M0072-G06, germlined M0064-E04, germlined M0064-F02, and DX-2655.

2. The method of claim 1, wherein the antibody competes with or binds the same epitope as DX-2647.

3. The method of claim 1, wherein the antibody competes with or binds the same epitope as M0064-F02.

4. A method of treating or preventing systemic sclerosis-associated pulmonary fibrosis in a subject, the method comprising:
administering an isolated protein comprising a heavy chain immunoglobulin variable domain sequence and a light chain immunoglobulin variable domain sequence to the subject,
wherein:
the heavy chain immunoglobulin variable domain sequence comprises three CDR regions from the heavy chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or
the light chain immunoglobulin variable domain sequence comprises three CDR regions from the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively), and
the protein binds to and inhibits both IGF-II and IGF-IIE.

5. The method of claim 4, wherein the three CDR regions from the heavy chain variable domain are from DX-2647 and/or the three CDR regions from the light chain variable domain are from DX-2647.

6. The method of claim 4, wherein the heavy chain immunoglobulin variable domain sequence comprises the heavy chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain immunoglobulin variable domain sequence comprises the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

7. The method of claim 4, wherein the heavy chain immunoglobulin variable domain sequence comprises the heavy chain variable domain of DX-2647, and/or the light chain immunoglobulin variable domain sequence comprises the light chain variable domain of DX-2647.

8. The method of claim 4, wherein the protein comprises the heavy chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

9. The method of claim 4, wherein the protein comprises the heavy chain of DX-2647, and/or the light chain of DX-2647.

10. A method of treating or preventing systemic sclerosis-associated pulmonary fibrosis in a subject, the method comprising:
administering to the subject an isolated protein comprising a heavy chain immunoglobulin variable domain sequence and a light chain immunoglobulin variable domain sequence, wherein
the heavy chain immunoglobulin variable domain sequence comprises three CDR regions from the heavy chain variable domain of M0080-G03 or M0073-C11, and/or
the light chain immunoglobulin variable domain sequence comprises three CDR regions from the heavy chain variable domain of M0080-G03 or M0073-C11 (respectively),
and the protein binds to and inhibits IGF-IIE but not IGF-II.

11. The method of claim 10, wherein the heavy chain immunoglobulin variable domain sequence comprises the heavy chain variable domain of M0080-G03 or M0073-C11, and/or the light chain immunoglobulin variable domain sequence comprises the light chain variable domain of M0080-G03 or M0073-C11 (respectively).

12. The method of claim 10, wherein the protein comprises the heavy chain of M0080-G03 or M0073-C11, and/or the light chain of M0080-G03 or M0073-C11 (respectively).

13. A method of treating or preventing systemic sclerosis-associated pulmonary fibrosis in a subject, the method comprising:
administering to the subject an isolated protein capable of specifically binding to the following consensus sequence or a functional fragment thereof:
TXCGGXLVXXLXXXXXXXXFXXXXPXXRVXRXSRGXVEEXCFRXXXXXXX
XXY
wherein X is any amino acid.

14. The method of claim 13, wherein the protein is capable of specifically binding to the following consensus sequence or a functional fragment thereof:
SETLCGGELVDTLQFVCGDRGFYFSRPASRVSRRSRGIVEECCFR
SCDLALLETYCATPA.

15. The method of claim 13, wherein the protein comprises a heavy chain immunoglobulin variable domain sequence and a light chain immunoglobulin variable domain, wherein:
the heavy chain immunoglobulin variable domain sequence comprises three CDR regions from the heavy chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or
the light chain immunoglobulin variable domain sequence comprises three CDR regions from the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively), and
the protein binds to and inhibits both IGF-II and IGF-IIE.

16. The method of claim 13, wherein the three CDR regions from the heavy chain variable domain are from DX-2647 and/or the three CDR regions from the light chain variable domain are from DX-2647.

17. The method of claim 13, wherein the heavy chain immunoglobulin variable domain sequence comprises the heavy chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain immunoglobulin variable domain sequence comprises the light chain variable domain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

18. The method of claim 13, wherein the heavy chain immunoglobulin variable domain sequence comprises the heavy chain variable domain of DX-2647, and/or the light chain immunoglobulin variable domain sequence comprises the light chain variable domain of DX-2647.

19. The method of claim 13, wherein the protein comprises the heavy chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655, and/or the light chain of DX-2647, M0033-E05, M0063-F02, M0064-E04, M0064-F02, M0068-E03, M0070-H08, M0072-006, M0072-E03, M0072-G06, germlined M0064-E04, germlined M0064-F02, or DX-2655 (respectively).

20. The method of claim 13, wherein the protein comprises the heavy chain of DX-2647, and/or the light chain of DX-2647.


SEQUENCE LISTING (102 sequences)

SEQ ID NO: 1 - protein, 53 aa, Artificial Sequence
  Description of Artificial Sequence Synthetic consensus sequence
TXCGGXLVXXLXXXXXXXXFXXXXPXXRVXRXSRGXVEEXCFRXXXXXXXXXY

SEQ ID NO: 2 - protein, 60 aa, Homo sapiens
SETLCGGELVDTLQFVCGDRGFYFSRPASRVSRRSRGIVEECCFRSCDLALLETYCATPA

SEQ ID NO: 3 - DNA, 201 bp, Homo sapiens
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 4 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 5 - DNA, 201 bp, Mus musculus
gcttacggccccggagagactctgtgcggaggggagcttgttgacacgcttcagtttgtctgttcggaccgcggcttctacttcagcaggccttcaagccgtgccaaccgtcgcagccgtggcatcgtggaagagtgctgcttccgcagctgcgacctggccctcctggagacatactgtgccacccccgccaagtccgag

SEQ ID NO: 6 - protein, 67 aa, Mus musculus
AYGPGETLCGGELVDTLQFVCSDRGFYFSRPSSRANRRSRGIVEECCFRSCDLALLETYCATPAKSE

SEQ ID NO: 7 - DNA, 540 bp, Homo sapiens
atgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaag

SEQ ID NO: 8 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 9 - DNA, 540 bp, Mus musculus
atggggatcccagtggggaagtcgatgttggtgcttctcatctctttggccttcgccttgtgctgcatcgctgcttacggccccggagagactctgtgcggaggggagcttgttgacacgcttcagtttgtctgttcggaccgcggcttctacttcagcaggccttcaagccgtgccaaccgtcgcagccgtggcatcgtggaagagtgctgcttccgcagctgcgacctggccctcctggagacatactgtgccacccccgccaagtccgagagggacgtgtctacctctcaggccgtacttccggacgacttccccagataccccgtgggcaagttcttccaatatgacacctggagacagtccgcgggacgcctgcgcagaggcctgcctgccctcctgcgtgcccgccggggtcgcatgcttgccaaagagctcaaagagttcagagaggccaaacgtcatcgtcccctgatcgtgttaccacccaaagaccccgcccacgggggagcctcttcggagatgtccagcaaccatcag

SEQ ID NO: 10 - protein, 180 aa, Mus musculus
MGIPVGKSMLVLLISLAFALCCIAAYGPGETLCGGELVDTLQFVCSDRGFYFSRPSSRANRRSRGIVEECCFRSCDLALLETYCATPAKSERDVSTSQAVLPDDFPRYPVGKFFQYDTWRQSAGRLRRGLPALLRARRGRMLAKELKEFREAKRHRPLIVLPPKDPAHGGASSEMSSNHQ

SEQ ID NO: 11 - DNA, 324 bp, Artificial Sequence
  Description of Artificial Sequence Synthetic polynucleotide
caagacatccagatgacccagtctccatcctccctgtctgcatctgtaggagacagagtcaccatcacttgccgggcaagtcagcccattaacacatatttaaattggtatcagcagagaccagggaaagcccctaggatcctgatctatacttcatccactttgcaaagtggggtcccatcaaggttcagtggcagtggatctgggacagatttcactctcaccatcagcagtgtgcaacctgaagattttgcaacttactactgtcaacagagttacagtttccctctcactttcggcggagggaccaaggtggagatcaaa

SEQ ID NO: 12 - DNA, 327 bp, Artificial Sequence
  Description of Artificial Sequence Synthetic polynucleotide
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 13 - DNA, 360 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctaagtacgagatggattgggttcgccaagctcctggtaaaggtttggagtgggtttctgttatctcttcttctggtggcggtactatttatgctgactccgttaaaggtcgcttcactatctctagagacaactctaagaatactctctacttgcagatgaacagcttaagggctgaggacaccgccatgtattactgtgcgagaggccggaccctatacggaggtgcttttgatatctggggccaagggacaatggtcaccgtctcaagc

SEQ ID NO: 14 - DNA, 411 bp, Artificial Sequence
  Description of Artificial Sequence Synthetic polynucleotide
gcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcagaggctagttctgctagtaacgcg

SEQ ID NO: 15 - DNA, 324 bp, Artificial Sequence
  Description of Artificial Sequence Synthetic polynucleotide
caagacatccagatgacccagtctccatcctccctgtctgcatctgtaggagacagagtcaccatcacttgccaggcgagtcacgacattagcaactatttaaattggtatcagcagaaaccagggaaagcccctaagctcctgatttatgctgcatcccgtttgcaaagtggggtcccatcaaggttcagtggcggtggatctgggacagatttcagtctcaccatcagcagtctgcaagctgaagattttgcaacttattactgtcaacagagttacagtttccctcgaacttttggccaggggaccaacctggagatcaaa

SEQ ID NO: 16 - DNA, 327 bp, Artificial Sequence
  Description of Artificial Sequence Synthetic polynucleotide
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagtaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 17 - DNA, 366 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctgtttacgatatgaattgggttcgccaagctcctggtaaaggtttggagtgggtttcttctatctcttcttctggtggcggtactctttatgctgactccgttaaaggtcgcttcactatctctagagacaactctaagaatactctctacttgcagatgaacagcttaagggctgaggacacggctgtgtattactgtgcgagagactctgataccagttcttattactggtactacgatctctggggccgcggcaccctggtcaccgtctcaagc

SEQ ID NO: 18 - DNA, 417 bp, Artificial Sequence
  Description of Artificial Sequence Synthetic polynucleotide
gcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcagaggctagttctgctagtaacgcgtgatga

SEQ ID NO: 19 - DNA, 324 bp, Artificial Sequence
  Description of Artificial Sequence Synthetic polynucleotide
caagacatccagatgacccagtctccatcctcactgtctgcatctgttggagacagagtcaccatcagttgtcgggcaagtcagggcattaccaattatttagtctggtttcagcagaaaccagggaaagcccctaggctcctgatctatgatgcctccactttggaaagtggggtcccatcaaggttcagcggcagtggatctgggacagatttcactctcaccatcagcagcctgcagcctgaagattttgcaacttactattgtcaacaggctgacggtttccctctcactttcggcgaggggaccaaggtggagatgaaa

SEQ ID NO: 20 - DNA, 375 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctcattactctatgtggtgggttcgccaagctcctggtaaaggtttggagtgggtttcttatatcggtccttctggtggccatactcgttatgctgactccgttaaaggtcgcttcactatctctagagacaactctaagaatactctctacttgcagatgaacagcttaagggctgaggacacggccgtgtattactgtgctagagggctatattattatgatagtagtagcgtgactcatgcctttgatctctggggccaagggacaatggtcaccgtctcaagc

SEQ ID NO: 21 - DNA, 324 bp, Artificial Sequence
  Description of Artificial Sequence Synthetic polynucleotide
caagacatccagatgacccagtctccgtcctccctgtctgcatctgcaggagacagagtcaccatcacttgccgggcaagtcagagcattagcagttatttaaattggtatcagcagaaaccaggaaaagcccctaacctcctgatctatactacatccaatttacaaggtggggtcccatcaaggttcagtggcagtggatctgggacagatttcactctcaccatcagcagtctgcaacctgaagattttgcaacttactactgtcaacagggttacagtttccctctcactttcggcggagggaccaaggtagagatcaaa

SEQ ID NO: 22 - DNA, 381 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctcgttactggatgatttgggttcgccaagctcctggtaaaggtttggagtgggtttcttctatccgttcttctggcgagactaagtatgctgactccgttaaaggtcgcttcactatctctagagacaactctaagaatactctctacttgcagatgaacagcttaagggctgaggacacggccgtgtattactgtgcgagaggccctttaagcgattactatgatagtagtggttattactttgatgcttttgatatctggggccaagggacaatggtcaccgtctcaagc

SEQ ID NO: 23 - DNA, 327 bp, Artificial Sequence
  Description of Artificial Sequence Synthetic polynucleotide
caagacatccagatgacccagtctccatcctccctgtctgcatctgtaggagacagagtcaccatcacttgccgggcaagtcagagtattcgcaactatttaaattggtatcagcagaaaccagggaaagcccctaaactcctgatctatgctgcatccaagttggaagacggggtcccatcaagattcagtggcagtggaactgggacagatttcactctcaccatcagaagtctgcaacctgaagattttgcaagttatttctgtcaacagagctactctagtccagggatcactttcggccctgggaccaaggtggagatcaaa

SEQ ID NO: 24 - DNA, 357 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctgcttacattatgacttgggttcgccaagctcctggtaaaggtttggagtgggtttcttctatctctccttctggtggctatactgtttatgctgactccgttaaaggtcgcttcactatctctagagacaactctaagaatactctctacttgcagatgaacagcttaagggctgaggacacggccgtatattactgtgcgagagactcggggttcggggacccctttgactactggggccaagggacaatggtcaccgtctcaagc

SEQ ID NO: 25 - DNA, 324 bp, Artificial Sequence
  Description of Artificial Sequence Synthetic polynucleotide
caagacatccagatgacccagtctccatcctccctgtctgcatctgtaggagacagagtcaccatcacttgccgggcaagtcagagcattagcaattacttgaattggtatcaacagaaaccaggtaaagcccctaagctcctgatctatactgcatccactttgcagagtggggtcccatcaaggttcagtggcagtgcatctgggacagacttcactctcaccatcaacagtctgcaacctgaagattttgctacttactcctgtcaacagagttacaattccccctggacgttcggccaagggaccaaggtggaaatcaaa

SEQ ID NO: 26 - DNA, 366 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctaattacattatgtggtgggttcgccaagctcctggtaaaggtttggagtgggtttctgttatctcttcttctggtggcatgactcgttatgctgactccgttaaaggtcgcttcactatctctagagacaactctaagaatactctctacttgcagatgaacagcttaagggctgaggacacggccgtgtattactgtgcgagagataacggtgactacgtaggcgaaaaaggttttgatatctggggccaagggacaatggtcaccgtctcaagc

SEQ ID NO: 27 - DNA, 324 bp, Artificial Sequence
  Description of Artificial Sequence Synthetic polynucleotide
caagacatccagatgacccagtctccatcctccctgtctgcatctgtaggagacagagtcaccatcacttgccgggcaagtcagagcattaacacttatttaaattggtatcagcagaaaccagggaaagcccctaaggtcctgatccatgctgcatccactttggaaagtggggtgccatcaaggttcagtggcagtggatctgcgacagaattcactctcaccatcagcagtctgcaacctgaagattttgcaactttctactgtcaacagagttacagtgtgcccttcactttcggccctgggaccagactgtctagcaaa

SEQ ID NO: 28 - DNA, 360 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctgagtacgttatggcttgggttcgccaagctcctggtaaaggtttggagtgggtttcttctatcgtttcttctggtggctatactaagtatgctgactccgttaaaggtcgcttcactatctctagagacaactctaagaatactctctacttgcagatgaacagcttaagggctgaggacacggccgtgtattactgtgcaaaagatatgacttacagtggggatgcttttgatgtctggggccaagggacaatggtcaccgtctcaagc

SEQ ID NO: 29 - DNA, 324 bp, Artificial Sequence
  Description of Artificial Sequence Synthetic polynucleotide
caagacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttatccgtggacgttcggccaagggaccaaggtggaaatcaaa

SEQ ID NO: 30 - DNA, 378 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctgagtacgagatgtcttgggttcgccaagctcctggtaaaggtttggagtgggtttcttctatctattcttctggtggctggactaagtatactgactccgttaaaggtcgcttcactatctctagagacaactctaagaatactctctacttgcagatgaacagcttaagggctgaggacacggccgtgtattactgtgcgaaaggtgtacactatgatagtagtggccttcctattgactggtacttcgatctctggggccgtggcaccctggtcaccgtctcaagc

SEQ ID NO: 31 - DNA, 324 bp, Artificial Sequence
  Description of Artificial Sequence Synthetic polynucleotide
caagacatccagatgacccagtctccttccaccctgtctgcatctgtaggagacagagtcaccatcacttgccgggccagtcagacaattagtagctggctggcctggtatcagcagaaaccagggaaagcccctaagttgatgatctataaggcggctagtttaggaagtgaggtcccatcaaggttcagcggcagtggatctgggacagagttcactctcaccatcagcagcctgcagcctgaagatttttcaacttactactgccaacagtacaaaacttatcccgtcacttttggccaggggaccaggctggagatcaaa

SEQ ID NO: 32 - DNA, 357 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctgagtacgttatgtggtgggttcgccaagctcctggtaaaggtttggagtgggtttctgttatctctccttctggtggctatactgtttatgctgactccgttaaaggtcgcttcactatctctagagacaactctaagaatactctctacttgcagatgaacagtttaagggctgaggacacggccgtgtattactgtgcgagagatcgggggggagctactacccttgactactggggccagggaaccctggtcaccgtctcaagc

SEQ ID NO: 33 - protein, 108 aa, Artificial Sequence
  Description of Artificial Sequence Synthetic polypeptide
QDIQMTQSPSSLSASVGDRVTITCRASQPINTYLNWYQQRPGKAPRILIYTSSTLQSGVPSRFSGSGSGTDFTLTISSVQPEDFATYYCQQSYSFPLTFGGGTKVEIK

SEQ ID NO: 34 - protein, 107 aa, Artificial Sequence [sequence omitted]

SEQ ID NO: 35 - protein, 120 aa, Artificial Sequence
  Description of Artificial Sequence Synthetic polypeptide
EVQLLESGGGLVQPGGSLRLSCAASGFTFSKYEMDWVRQAPGKGLEWVSVISSSGGGTIYADSVKGRFTISRDNSKNTLYLQMNSLRAEDTAMYYCARGRTLYGGAFDIWGQGTMVTVSS

SEQ ID NO: 36 - protein, 137 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAAEASSASNA

SEQ ID NO: 37 - protein, 108 aa, Artificial Sequence
  Description of Artificial Sequence Synthetic polypeptide
QDIQMTQSPSSLSASVGDRVTITCQASHDISNYLNWYQQKPGKAPKLLIYAASRLQSGVPSRFSGGGSGTDFSLTISSLQAEDFATYYCQQSYSFPRTFGQGTNLEIK

SEQ ID NO: 38 - protein, 122 aa, Artificial Sequence
  Description of Artificial Sequence Synthetic polypeptide
EVQLLESGGGLVQPGGSLRLSCAASGFTFSVYDMNWVRQAPGKGLEWVSSISSSGGGTLYADSVKGRFTISRDNSKNTLYLQMNSLRAEDTAVYYCARDSDTSSYYWYYDLWGRGTLVTVSS

SEQ ID NO: 39 - protein, 108 aa, Artificial Sequence
  Description of Artificial Sequence Synthetic polypeptide
QDIQMTQSPSSLSASVGDRVTISCRASQGITNYLVWFQQKPGKAPRLLIYDASTLESGVPSRFSGSGSGTDFTLTISSLQPEDFATYYCQQADGFPLTFGEGTKVEMK

SEQ ID NO: 40 - protein, 125 aa, Artificial Sequence
  Description of Artificial Sequence Synthetic polypeptide
EVQLLESGGGLVQPGGSLRLSCAASGFTFSHYSMWWVRQAPGKGLEWVSYIGPSGGHTRYADSVKGRFTISRDNSKNTLYLQMNSLRAEDTAVYYCARGLYYYDSSSVTHAFDLWGQGTMVTVSS

SEQ ID NO: 41 - protein, 108 aa, Artificial Sequence
  Description of Artificial Sequence Synthetic polypeptide
QDIQMTQSPSSLSASAGDRVTITCRASQSISSYLNWYQQKPGKAPNLLIYTTSNLQGGVPSRFSGSGSGTDFTLTISSLQPEDFATYYCQQGYSFPLTFGGGTKVEIK

SEQ ID NO: 42 - protein, 127 aa, Artificial Sequence
  Description of Artificial Sequence Synthetic polypeptide
EVQLLESGGGLVQPGGSLRLSCAASGFTFSRYWMIWVRQAPGKGLEWVSSIRSSGETKYADSVKGRFTISRDNSKNTLYLQMNSLRAEDTAVYYCARGPLSDYYDSSGYYFDAFDIWGQGTMVTVSS

SEQ ID NO: 43 - protein, 109 aa, Artificial Sequence
  Description of Artificial Sequence Synthetic polypeptide
QDIQMTQSPSSLSASVGDRVTITCRASQSIRNYLNWYQQKPGKAPKLLIYAASKLEDGVPSRFSGSGTGTDFTLTIRSLQPEDFASYFCQQSYSSPGITFGPGTKVEIK

SEQ ID NO: 44 - protein, 119 aa, Artificial Sequence
  Description of Artificial Sequence Synthetic polypeptide
EVQLLESGGGLVQPGGSLRLSCAASGFTFSAYIMTWVRQAPGKGLEWVSSISPSGGYTVYADSVKGRFTISRDNSKNTLYLQMNSLRAEDTAVYYCARDSGFGDPFDYWGQGTMVTVSS

SEQ ID NO: 45 - protein, 108 aa, Artificial Sequence
  Description of Artificial Sequence Synthetic polypeptide
QDIQMTQSPSSLSASVGDRVTITCRASQSISNYLNWYQQKPGKAPKLLIYTASTLQSGVPSRFSGSASGTDFTLTINSLQPEDFATYSCQQSYNSPWTFGQGTKVEIK

SEQ ID NO: 46 - protein, 122 aa, Artificial Sequence
  Description of Artificial Sequence Synthetic polypeptide
EVQLLESGGGLVQPGGSLRLSCAASGFTFSNYIMWWVRQAPGKGLEWVSVISSSGGMTRYADSVKGRFTISRDNSKNTLYLQMNSLRAEDTAVYYCARDNGDYVGEKGFDIWGQGTMVTVSS

SEQ ID NO: 47 - protein, 108 aa, Artificial Sequence
  Description of Artificial Sequence Synthetic polypeptide
QDIQMTQSPSSLSASVGDRVTITCRASQSINTYLNWYQQKPGKAPKVLIHAASTLESGVPSRFSGSGSATEFTLTISSLQPEDFATFYCQQSYSVPFTFGPGTRLSSK

SEQ ID NO: 48 - protein, 120 aa, Artificial Sequence
  Description of Artificial Sequence Synthetic polypeptide
EVQLLESGGGLVQPGGSLRLSCAASGFTFSEYVMAWVRQAPGKGLEWVSSIVSSGGYTKYADSVKGRFTISRDNSKNTLYLQMNSLRAEDTAVYYCAKDMTYSGDAFDVWGQGTMVTVSS

SEQ ID NO: 49 - protein, 108 aa, Artificial Sequence
  Description of Artificial Sequence Synthetic polypeptide
QDIQMTQSPSTLSASVGDRVTITCRASQSISSWLAWYQQKPGKAPKLLIYKASSLESGVPSRFSGSGSGTEFTLTISSLQPDDFATYYCQQYNSYPWTFGQGTKVEIK

SEQ ID NO: 50 - protein, 126 aa, Artificial Sequence
  Description of Artificial Sequence Synthetic polypeptide
EVQLLESGGGLVQPGGSLRLSCAASGFTFSEYEMSWVRQAPGKGLEWVSSIYSSGGWTKYTDSVKGRFTISRDNSKNTLYLQMNSLRAEDTAVYYCAKGVHYDSSGLPIDWYFDLWGRGTLVTVSS

SEQ ID NO: 51 - protein, 108 aa, Artificial Sequence
  Description of Artificial Sequence Synthetic polypeptide
QDIQMTQSPSTLSASVGDRVTITCRASQTISSWLAWYQQKPGKAPKLMIYKAASLGSEVPSRFSGSGSGTEFTLTISSLQPEDFSTYYCQQYKTYPVTFGQGTRLEIK

SEQ ID NO: 52 - protein, 119 aa, Artificial Sequence
  Description of Artificial Sequence Synthetic polypeptide
EVQLLESGGGLVQPGGSLRLSCAASGFTFSEYVMWWVRQAPGKGLEWVSVISPSGGYTVYADSVKGRFTISRDNSKNTLYLQMNSLRAEDTAVYYCARDRGGATTLDYWGQGTLVTVSS

SEQ ID NO: 53 - protein, 91 aa, Artificial Sequence
  Description of Artificial Sequence Synthetic polypeptide
DIQMTQSPSSLSASVGDRVTITCQASHDISNYLNWYQQKPGKAPKLLIYAASRLQSRFSGGGSGTDFSLTISSLQAEDFATYYCQQSYSFP

SEQ ID NO: 54 - protein, 91 aa, Artificial Sequence
  Description of Artificial Sequence Synthetic polypeptide
DIQMTQSPSSLSASVGDRVTITCRASQSISSYLNWYQQKPGKAPKLLIYAASSLQSRFSGSGSGTDFTLTISSLQPEDFATYYCQQSYSTP

SEQ ID NO: 55 - protein, 11 aa, Artificial Sequence
  Description of Artificial Sequence Synthetic peptide
TFGQGTNLEIK

SEQ ID NO: 56 - protein, 11 aa, Artificial Sequence
  Description of Artificial Sequence Synthetic peptide
TFGQGTRLEIK

SEQ ID NO: 57 - protein, 91 aa, Artificial Sequence
  Description of Artificial Sequence Synthetic polypeptide
DIQMTQSPSSLSASVGDRVTITCRASQSISNYLNWYQQKPGKAPKLLIYTASTLQSRFSGSASGTDFTLTINSLQPEDFATYSCQQSYNSP

SEQ ID NO: 58 - protein, 12 aa, Artificial Sequence
  Description of Artificial Sequence Synthetic peptide
WTFGQGTKVEIK

SEQ ID NO: 59 - DNA, 339 bp, Artificial Sequence
  Description of Artificial Sequence Synthetic polynucleotide
cagagcgctttgactcagccaccctcagtgtccgtgtccccaggacagacagccagcatcacctgctctggagatagattgggggataaatatgcttcctggtatcagcagaagccaggccagtcccctgtgttggtcatctatcaagataccaagcggccctcagggatccctgagcgattctctggctccaactctgggtccacagccactctgaccatcagcgggacccaggctatggatgaggctgactattactgtcaggcgtgggacagcagcactgtggtattcggcggagggaccaagctgaccgtcctaggtcagcccaaggctgccccc

SEQ ID NO: 60 - DNA, 426 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctccttacgatatgtggtgggttcgccaagctcctggtaaaggtttggagtgggtttcttctatctcttcttctggtggcattactgcttatgctgactccgttaaaggtcgcttcactatctctagagacaactctaagaatactctctacttgcagatgaacagcttaagggctgaggacacggccgtgtattactgtgcgagggccgggtatagcagcagctgggggtactactactacggtatggacgtctggggccaagggaccacggtcaccgtctcaagcgcctccaccaagggcccatcggtcttcccgctagcaccctcctccaagagc

SEQ ID NO: 61 - protein, 113 aa, Artificial Sequence
  Description of Artificial Sequence Synthetic polypeptide
QSALTQPPSVSVSPGQTASITCSGDRLGDKYASWYQQKPGQSPVLVIYQDTKRPSGIPERFSGSNSGSTATLTISGTQAMDEADYYCQAWDSSTVVFGGGTKLTVLGQPKAAP

SEQ ID NO: 62 - protein, 142 aa, Artificial Sequence
  Description of Artificial Sequence Synthetic polypeptide
EVQLLESGGGLVQPGGSLRLSCAASGFTFSPYDMWWVRQAPGKGLEWVSSISSSGGITAYADSVKGRFTISRDNSKNTLYLQMNSLRAEDTAVYYCARAGYSSSWGYYYYGMDVWGQGTTVTVSSASTKGPSVFPLAPSSKS

SEQ ID NO: 63 - DNA, 351 bp, Artificial Sequence
  Description of Artificial Sequence Synthetic polynucleotide
cagagcgtcttgactcagccaccctcagcgtctgggacccccgggcagagggtcaccatctcttgttctggaagcagctccaacatcggaagttattatgtatattggtaccagcagatcccaggaacggcccccaaactcctcatctataggaataatcagcggccctcaggggtccctgaccgattctctggcgccaagtctggcacctcagcctccctggccatcagtgggctccggtccgaggatgaggctgattattactgtgcagcatgggatgacagcctccatggttgggttttcggcggagggaccaagctgaccgtcctaggtcagcccaaggctgccccc

SEQ ID NO: 64 - DNA, 438 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctttttaccagatgatgtgggttcgccaagctcctggtaaaggtttggagtgggtttcttctatctctccttctggtggccgtacttattatgctgactccgttaaaggtcgcttcactatctctagagacaactctaagaatactctctacttgcagatgaacagcttaagggctgaggacacggccgtgtattactgtgcaaaagatattgggctaggatattgtagtagtaccagctgctatacgggtacccctcttgactactggggccagggcaccctggtcaccgtctcaagcgcctccaccaagggcccatcggtcttcccgctagcaccctcctccaagagc

SEQ ID NO: 65 - protein, 117 aa, Artificial Sequence
  Description of Artificial Sequence Synthetic polypeptide
QSVLTQPPSASGTPGQRVTISCSGSSSNIGSYYVYWYQQIPGTAPKLLIYRNNQRPSGVPDRFSGAKSGTSASLAISGLRSEDEADYYCAAWDDSLHGWVFGGGTKLTVLGQPKAAP

SEQ ID NO: 66 - protein, 146 aa, Artificial Sequence
  Description of Artificial Sequence Synthetic polypeptide
EVQLLESGGGLVQPGGSLRLSCAASGFTFSFYQMMWVRQAPGKGLEWVSSISPSGGRTYYADSVKGRFTISRDNSKNTLYLQMNSLRAEDTAVYYCAKDIGLGYCSSTSCYTGTPLDYWGQGTLVTVSSASTKGPSVFPLAPSSKS

SEQ ID NO: 67 - protein, 214 aa, Artificial Sequence
  Description of Artificial Sequence Synthetic polypeptide
DIQMTQSPSSLSASVGDRVTITCRASQSISNYLNWYQQKPGKAPKLLIYTASTLQSGVPSRFSGSGSGTDFTLTISSLQPEDFATYYCQQSYNSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 452 aa, Artificial Sequence
  Description of Artificial Sequence Synthetic polypeptide
EVQLLESGGGLVQPGGSLRLSCAASGFTFSNYIMWWVRQAPGKGLEWVSVISSSGGGTLYADSVKGRFTISRDNSKNTLYLQMNSLRAEDTAVYYCARDNGDYVGEKGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 642 bp, Artificial Sequence
  Description of Artificial Sequence Synthetic polynucleotide
gacatccagatgacccagtcccccagctccctgtctgcttccgtgggcgaccgggtgaccatcacctgccgggcctcccagtccatctccaactacctgaactggtatcagcagaagcctggcaaggcccctaagctgctgatctacaccgcctctacactgcagtctggagtcccttccaggttctccggctccggcagcggcaccgacttcaccctgaccatctcctccctgcagcctgaggacttcgccacctactactgccagcagtcctacaactccccttggaccttcggccagggcaccaaggtggagatcaagcggaccgtggccgctccttccgtgttcatcttccctccctccgacgagcagctgaaatccggcactgccagcgtggtctgcctgctgaacaacttctaccctcgggaggccaaggtgcagtggaaggtggacaacgccctgcagagcggcaactcccaggaatccgtcaccgagcaggactccaaggacagcacctactccctgtccagcaccctgaccctgtccaaggccgactacgagaagcacaaggtgtacgcctgcgaggtgacccaccagggcctgtcctcccccgtgaccaagtccttcaaccggggcgagtgc

SEQ ID NO: 70 - DNA, 1356 bp, Artificial Sequence
  Description of Artificial Sequence Synthetic polynucleotide
gaggtgcaattgctggagtctggcggcggactggtgcagcctggcggctccctgcggctgtcctgcgccgcctccggcttcaccttctccaactacatcatgtggtgggtgcggcaggctcctggaaagggcctcgagtgggtgtccgtgatctccagctccgggggaggaacactgtacgccgactccgtgaagggccggttcaccatctccagagacaactccaagaacaccctgtacctgcagatgaactccctgcgggccgaggacaccgccgtgtactactgcgccagggacaacggcgactacgtgggcgagaagggcttcgacatctggggccagggcacaatggtgaccgtgtcctccgcctccaccaagggcccttccgtgttcccgctagcaccttcctccaagtccacctctggcggcaccgccgctctgggctgcctggtgaaggactacttccctgagcctgtgaccgtgagctggaactctggcgccctgacctccggcgtgcataccttccctgccgtgctgcagtcctccggcctgtactccctgtcctccgtggtgacagtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccctccgtgttcctgttccctcctaagcctaaggacaccctgatgatctcccggacccctgaggtgacctgcgtggtggtggacgtgtcccacgaggacccagaggtgaagtttaattggtatgtggacggcgtggaggtccacaacgccaagaccaagcctcgggaggaacagtacaactccacctaccgggtggtgtccgtgctgaccgtgctgcaccaggactggctgaacggcaaggaatacaagtgcaaagtctccaacaaggccctgcctgcccccatcgagaaaaccatctccaaggccaagggccagcctcgcgagcctcaggtgtacaccctgcctcctagccgggaggaaatgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgagccctggcaag

SEQ ID NO: 71 - protein, 23 aa, Artificial Sequence
  Description of Artificial Sequence Synthetic peptide
DIQMTQSPSSLSASVGDRVTITC

SEQ ID NO: 72 - protein, 11 aa, Artificial Sequence
  Description of Artificial Sequence Synthetic peptide
RASQSISNYLN

SEQ ID NO: 73 - protein, 15 aa, Artificial Sequence
  Description of Artificial Sequence Synthetic peptide
WYQQKPGKAPKLLIY

SEQ ID NO: 74 - protein, 7 aa, Artificial Sequence
  Description of Artificial Sequence Synthetic peptide
TASTLQS

SEQ ID NO: 75 - protein, 32 aa, Artificial Sequence
  Description of Artificial Sequence Synthetic polypeptide
GVPSRFSGSGSGTDFTLTISSLQPEDFATYYC

SEQ ID NO: 76 - protein, 9 aa, Artificial Sequence
  Description of Artificial Sequence Synthetic peptide
QQSYNSPWT

SEQ ID NO: 77 - protein, 10 aa, Artificial Sequence
  Description of Artificial Sequence Synthetic peptide
FGQGTKVEIK

SEQ ID NO: 78 - protein, 30 aa, Artificial Sequence
  Description of Artificial Sequence Synthetic polypeptide
EVQLLESGGGLVQPGGSLRLSCAASGFTFS

SEQ ID NO: 79 - protein, 5 aa, Artificial Sequence
  Description of Artificial Sequence Synthetic peptide
NYIMW

SEQ ID NO: 80 - protein, 14 aa, Artificial Sequence
  Description of Artificial Sequence Synthetic peptide
WVRQAPGKGLEWVS

SEQ ID NO: 81 - protein, 17 aa, Artificial Sequence
  Description of Artificial Sequence Synthetic peptide
VISSSGGGTLYADSVKG

SEQ ID NO: 82 - protein, 32 aa, Artificial Sequence
  Description of Artificial Sequence Synthetic polypeptide
RFTISRDNSKNTLYLQMNSLRAEDTAVYYCAR

SEQ ID NO: 83 - protein, 13 aa, Artificial Sequence
  Description of Artificial Sequence Synthetic peptide
DNGDYVGEKGFDI

SEQ ID NO: 84 - protein, 11 aa, Artificial Sequence
  Description of Artificial Sequence Synthetic peptide
WGQGTMVTVSS

SEQ ID NO: 85 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 107 aa, Artificial Sequence
  Description of Artificial Sequence Synthetic polypeptide
DIQMTQSPSSLSASVGDRVTITCRASQSISNYLNWYQQKPGKAPKLLIYTASTLQSGVPSRFSGSGSGTDFTLTISSLQPEDFATYYCQQSYNSPWTFGQGTKVEIK

SEQ ID NO: 87 - protein, 122 aa, Artificial Sequence
  Description of Artificial Sequence Synthetic polypeptide
EVQLLESGGGLVQPGGSLRLSCAASGFTFSNYIMWWVRQAPGKGLEWVSVISSSGGGTLYADSVKGRFTISRDNSKNTLYLQMNSLRAEDTAVYYCARDNGDYVGEKGFDIWGQGTMVTVSS

SEQ ID NO: 88 - protein, 32 aa, Artificial Sequence
  Description of Artificial Sequence Synthetic polypeptide
GVPSRFSGSASGTDFTLTINSLQPEDFATYSC

SEQ ID NO: 89 - protein, 214 aa, Artificial Sequence
  Description of Artificial Sequence Synthetic polypeptide
DIQMTQSPSSLSASVGDRVTITCQASHDISNYLNWYQQKPGKAPKLLIYAASRLQSGVPSRFSGSGSGTDFTLTISSLQPEDFATYYCQQSYSFPRTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 452 aa, Artificial Sequence
  Description of Artificial Sequence Synthetic polypeptide
EVQLLESGGGLVQPGGSLRLSCAASGFTFSVYDMNWVRQAPGKGLEWVSSISSSGGGTLYADSVKGRFTISRDNSKNTLYLQMNSLRAEDTAVYYCARDSDTSSYYWYYDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 642 bp, Artificial Sequence
  Description of Artificial Sequence Synthetic polynucleotide
gatatccagatgacccagtcccccagctccctgtccgctagcgtgggcgaccgggtgaccatcacctgccaggcctcccacgacatctccaactacctgaactggtatcagcagaagcctggcaaggcccctaagctgctgatctacgccgccagcagactgcagtccggcgtccctagccggttctccggctccggcagcggcaccgacttcaccctgaccatctcctccctgcagcctgaggacttcgccacctactactgccagcagtcctactccttccctcggaccttcggccagggcacccggctggagatcaagcggaccgtggccgctccttccgtgttcatcttccctccctccgacgagcagctgaagagcggcacagccagcgtcgtgtgcctgctgaacaacttctaccctcgggaggccaaggtgcagtggaaggtggacaacgccctgcagagcggcaactcccaggaatccgtcaccgagcaggactccaaggacagcacctactccctgtcctccaccctgaccctgtccaaggccgactacgagaagcacaaggtgtacgcctgcgaggtgacccaccagggcctgtccagccctgtgaccaagtccttcaaccggggcgagtgc

SEQ ID NO: 92 - DNA, 1356 bp, Artificial Sequence
  Description of Artificial Sequence Synthetic polynucleotide
gaggtgcaattgctggagtctggcggcggactggtgcagcctggcggctccctgcggctgtcctgcgccgcctccggcttcaccttctccgtgtacgacatgaactgggtgcggcaggctcctggaaagggcctcgagtgggtgtcctccatctccagctccgggggaggaacactgtacgccgactccgtgaagggccggttcaccatctccagagacaactccaagaacaccctgtacctgcagatgaactccctgcgggccgaggacaccgccgtgtactactgcgccagggactccgacacctcctcctactactggtactacgacctgtggggcaggggcaccctggtgaccgtgtcctccgcctccaccaagggcccttccgtgttcccgctagcaccttcctccaagtccacctctggcggcaccgccgctctgggctgcctggtgaaggactacttccctgagcctgtgaccgtgagctggaactctggcgccctgacctccggcgtgcataccttccctgccgtgctgcagtcctccggcctgtactccctgtcctccgtggtgacagtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccctccgtgttcctgttccctcctaagcctaaggacaccctgatgatctcccggacccctgaggtgacctgcgtggtggtggacgtgtcccacgaggacccagaggtgaagtttaattggtatgtggacggcgtggaggtccacaacgccaagaccaagcctcgggaggaacagtacaactccacctaccgggtggtgtccgtgctgaccgtgctgcaccaggactggctgaacggcaaggaatacaagtgcaaagtctccaacaaggccctgcctgcccccatcgagaaaaccatctccaaggccaagggccagcctcgcgagcctcaggtgtacaccctgcctcctagccgggaggaaatgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgagccctggcaag

SEQ ID NO: 93 - protein, 11 aa, Artificial Sequence
  Description of Artificial Sequence Synthetic peptide
QASHDISNYLN

SEQ ID NO: 94 - protein, 7 aa, Artificial Sequence
  Description of Artificial Sequence Synthetic peptide
AASRLQS

SEQ ID NO: 95 - protein, 9 aa, Artificial Sequence
  Description of Artificial Sequence Synthetic peptide
QQSYSFPRT

SEQ ID NO: 96 - protein, 10 aa, Artificial Sequence
  Description of Artificial Sequence Synthetic peptide
FGQGTRLEIK

SEQ ID NO: 97 - protein, 5 aa, Artificial Sequence
  Description of Artificial Sequence Synthetic peptide
VYDMN

SEQ ID NO: 98 - protein, 17 aa, Artificial Sequence
  Description of Artificial Sequence Synthetic peptide
SISSSGGGTLYADSVKG

SEQ ID NO: 99 - protein, 13 aa, Artificial Sequence
  Description of Artificial Sequence Synthetic peptide
DSDTSSYYWYYDL

SEQ ID NO: 100 - protein, 11 aa, Artificial Sequence
  Description of Artificial Sequence Synthetic peptide
WGRGTLVTVSS

SEQ ID NO: 101 - protein, 32 aa, Artificial Sequence
  Description of Artificial Sequence Synthetic polypeptide
GVPSRFSGGGSGTDFSLTISSLQAEDFATYYC

SEQ ID NO: 102 - protein, 10 aa, Artificial Sequence
  Description of Artificial Sequence Synthetic peptide
FGQGTNLEIK